(19) United States
(12) Patent Application Publication
(10) Pub. No.: US 2024/0238441 A1
(43) Pub. Date: Jul. 18, 2024

(54) EXTRACELLULAR VESICLE-DIRECTED POLYPEPTIDE TAG

(71) Applicant: Ottawa Hospital Research Institute, Ottawa (CA)

(72) Inventors: Michael A. RUDNICKI, Ottawa (CA); Uxia GURRIARAN-RODRIGUEZ, Bilbao (ES); David Alexander DATZKIW, Ottawa (CA)

(21) Appl. No.: 18/558,669

(22) PCT Filed: May 3, 2022
(86) PCT No.: PCT/CA2022/050689

§ 371 Date: Nov. 2, 2023

Related U.S. Application Data
(60) Provisional application No. 63/183,902, filed on May 4, 2021.

(51) Int. Cl. A61K 47/69; A61K 45/06; A61K 47/62

(52) U.S. Cl. CPC A61K 47/6901; A61K 45/06; A61K 47/62; C07K 2319/055

(57) ABSTRACT

The present invention is based on the surprising finding that Wnt7a, and other Wnt family members, are trafficked to extracellular vesicles (EVs) via interactions with coatomer proteins. Extracellular vesicle signal peptides (ESPs), each comprising at least one key Coatomer binding motif (CBM), are described, and these mediate EV trafficking of Wnt family members. The ESPs may be used to target other proteins for display on EVs. Herein is described an EV comprising: COPI, and a recombinant EV-directed polypeptide comprising: a cargo polypeptide, and an ESP comprising a CBM, wherein the cargo polypeptide is tethered to an external surface of the EV via the coatomer binding motif. Also described are recombinant EV-directed polypeptides comprising an ESP and a cargo. Additionally described are recombinant skeletal muscle-targeted EVs comprising a pay load polypeptide; and recombinant Wnts having mutated CBM or ESPs, which are less trafficked to EVs for production/recovery of free Wnts.

### CROSS REFERENCE TO RELATED APPLICATIONS

This application claims priority from U.S. Provisional Application No. 63/183,902 entitled “EXTRACELLULAR VESICLE-DIRECTED POLYPEPTIDE TAG”, which was filed May 4, 2021, which is hereby incorporated by reference in its entirety.

### FIELD

The present disclosure relates generally to recombinant polypeptides. More particularly, the present disclosure relates to extracellular-vesicle targeted recombinant polypeptides.

### BACKGROUND

Extracellular vesicles (EVs) are lipid bilayer-delimited particles that are naturally released from a cell. Exosomes are 40-150 nm small extracellular vesicles (EVs) of endocytic origin involved in intercellular communication that transfer bioactive cargo, for example lipids, proteins, microRNAs, and mRNAs, to distal cells.

Because of their ability to function as an intercellular transfer system, EVs have been studied for use as potential vehicles delivery of therapeutic molecules. In addition, certain EVs also possess inherent therapeutic characteristics.

In order to understand how EVs can be used for therapeutic purposes, it is important to understand the processes by which they are formed and how they function in health and disease.

It would desirable to have additional ways of targeting molecules of interest to EVs.

### SUMMARY

It is an object of the present disclosure to obviate or mitigate at least one disadvantage of previous approaches.

In a first aspect, the present disclosure provides an extracellular vesicle (EV) comprising: coat protein complex 1 (COPI), and a recombinant EV-directed polypeptide comprising: a cargo polypeptide, and an extracellular vesicle signal peptide (ESP) comprising a coatomer binding motif (CBM), wherein the cargo polypeptide is tethered to an external surface of the EV via the coatomer binding motif.

In another aspect, there is provided a recombinant extracellular vesicle (EV)-directed polypeptide comprising: a cargo polypeptide, and an extracellular vesicle signal peptide (ESP) comprising a coatomer binding motif (CBM).

In another aspect, there is provided a nucleic acid molecule encoding the recombinant EV-directed polypeptide as described herein.

In another aspect, there is provided a viral particle comprising the nucleic acid as described herein.

In another aspect, there is provided a recombinant host cell comprising the nucleic acid as described herein.

In another aspect, there is provided a composition comprising the EV as described herein, the nucleic acid as described herein, or the viral particle as described herein; together with an excipient diluent, or carrier.

In another aspect, there is provided a use of the EV as described herein for delivery of the cargo polypeptide to a cell.

In another aspect, there is provided a use of the EV as described herein for preparation of a composition for delivery of the cargo polypeptide to a cell.

In another aspect, there is provided the EV as described herein for use in delivery of a cargo polypeptide to a cell.

In another aspect, there is provided a method of delivering a cargo polypeptide to a cell comprising contacting the cell with the EV as described herein.

In one aspect, there is provided a recombinant skeletal muscle-directed extracellular vesicle (EV) comprising coat protein complex 1 (COPI), a skeletal muscle targeting moiety comprising a Wnt family polypeptide, or polypeptide at least 90% identical thereto, and a payload for delivery to skeletal muscle.

In one aspect, there is provided a method for delivering a payload to skeletal muscle comprising contacting a cell with the recombinant skeletal muscle-directed EV as defined herein.

In one aspect, there is provided a use the recombinant skeletal muscle-directed EV as defined herein for delivery of the payload to skeletal muscle.

In one aspect, there is provided the recombinant skeletal muscle-directed EV as defined herein for use in delivery of the payload to skeletal muscle.

In one aspect, there is provided a recombinant Wnt protein comprising an extracellular vesicle signal peptide (ESP) sequence comprising one or more coatomer binding motifs (CBMs), wherein at least one of the one or more CBMs is mutated relative to a corresponding wild-type sequence to form a mutated CBM that reduces or abrogates extracellular vesicle-targeting activity of the ESP sequence relative to the corresponding wild-type sequence.

In one aspect, there is provided a recombinant nucleic acid encoding the recombinant Wnt protein as defined herein.

In one aspect, there is provided a vector comprising the recombinant nucleic acid as defined herein.

In one aspect, there is provided a host cell comprising the recombinant nucleic acid as defined here, or the vector as defined herein.

In one aspect, there is provided a use of the recombinant nucleic acid as defined here, or the host cell defined here, for production of the recombinant Wnt protein as defined herein, wherein the recombinant Wnt protein is free of extracellular vesicles.

In one aspect, there is provided a method for producing the recombinant Wnt protein as defined herein comprising introducing the recombinant nucleic acid as defined herein to a cell, and culturing the cell to produce the recombinant Wnt protein, wherein the recombinant Wnt protein is free of extracellular vesicles.

In one aspect, there is provided a method for producing the recombinant Wnt protein as defined herein comprising culturing the host cell as defined herein to produce the recombinant Wnt protein, wherein the recombinant Wnt protein is free of extracellular vesicles.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 shows immunogold transmission electron microscopy (ITEM) images of anti-Wnt7a labeling of new regenerating tibialis anterior myofibers at 96 h post-CTX injury from WT mice, showing Wnt7a secretion on exosomes contained in a multivesicular body (MVB) (upper panels). Scale bar 500 nm and 100 nm respectively. iTEM of anti-Wnt7a labeling of injured tibialis anterior muscle from WT mice, shows the reception of Wnt7a from extracellular vesicles (EVs) secreted by new regenerating myofibers (lower panels). Scale bar 500 nm and 100 nm, respectively.

FIG. 2 is a schematic representation of mouse strains used to generate conditional Wnt7a floxed in Myf5 expressing cells.

FIG. 3 shows iTEM images of anti-Wnt7a labeling of EVs showing abrogation of Wnt7a expression in EVs from Myf5^{(Cre/+)}:Wnt7a^{(fl/fl)} mice. Scale bar 100 nm.

FIG. 4 is a plot representing a hypertrophy assay of murine primary myotubes treated with EVs from muscle decreases hyper-trophy after Wnt7a abrogation. Data shown as fold change of myotube diameter over the control (%); Wnt7a recombinant protein was used as a positive control. (*p<0.05, ** p<0.005, *** p<0.0005).

FIG. 5 shows pan myosin heavy chain (pMHC) IF representative images of hypertrophied myoblasts after muscle EVs stimulation containing Wnt7a (n=3). Scale bar 50 μm.

FIG. 6 shows immunoblot EVs secretion analysis of Wnt7a serine palmitoylated mutants on cysteine 73 and serine 206, shows no interruption on EVs secretion upon single point mutation with alanine.

FIG. 7 shows immunoblot EVs secretion analysis of different Wnt7a truncates (right panel) shows interruption of secretion upon deletion beyond the n-terminus 100 aa amino acids and c-terminus 300 aa.

FIG. 8 shows immunoblot EVs secretion analysis of the minimal Wnt7a structure necessary for EVs secretion from 100-300 aa (right panel). Signal peptide is not required for EVs-Wnt7a secretion.

FIG. 9 shows the surface of Wnt7a with focus on the extracellular vesicles signal peptide (ESP), with negatively charged residues (originally red), positively charged residues (originally blue), and hydrophobic residues (originally green). The surface of the ESP shows its positive charge.

FIG. 10 shows immunoblot EVs secretion analysis, after ESP replacement with a linker domain (GSGS, right panel), exhibits the disruption of Wnt7a-EVs secretion and the displacement of Wnt7a secretion in favor of free protein secretion.

FIG. 11 shows immunoblot analysis of insertion of the ESP domain into an upstream domain of Wnt7a without perturbing stability of the full-length protein (Wnt7a-Δ3aa*GSG versus Wnt7a-Δ3aa*ESP). Insertion of ESP to this site (Wnt7a-Δ213-249*ESP) restores EVs localization to Wnt7a-Δ213-249.

FIG. 12 shows immunoblot EVs secretion analysis of the independence of structural location (c-terminal or n-terminal) of ESP to target Wnt7a-Δ213-249 into EVs.

FIG. 13 shows immunoblot EVs secretion analysis showing the ability of ESP to target HALO protein into EVs after fusion, independently of HA tag.

FIG. 14 shows an experimental schematic of the protocol to visualize HALO-ESP EVs uptake by Image Cytometry (top); and single cell analysis with Amnis ImageStream which detects the fluorescence inside the hosting HEK293T cells upon treatment with HALO-ESP EVs versus HALO EVs (bottom).

FIG. 15 shows a heat map displaying fold change (log 2 scale) of enriched proteins in mass spectrometry versus control conditions (ESP_BirA:BirA and Wnt7a_BirA:BirA). Shown are proteins that present a minimum enrichment of 50% (log 2 (FC)>0.5849) on ESP and a positive enrichment (log 2 (FC)>0) on Wnt7a. Found COPI complex subunits are highlighted in bold.

FIG. 16 shows Wnt7a: COPα PLA (originally red) performed in myotubes either expressing Wnt7a-BirA or BirA. PLA signal was counterstained with GM310 (originally green) and with DAPI (originally blue), evincing interaction in the Golgi area. Scale bar 10 μm.

FIG. 17 shows Wnt7a: COPβ2 PLA (originally red) performed in myotubes either expressing Wnt7a-BirA or BirA. PLA signal was counterstained with GM310 (originally green) and with DAPI (originally blue), showing interaction in the plasma membrane area. Scale bar 10 μm.

FIG. 18 shows HEK293T cells overexpressing Wnt7a-HA with immunoprecipitated COPβ2 antibody. Wnt7a-HA interacts with COPα and COPβ2.

FIG. 19 shows HEK293T cells overexpressing Wnt7a-HA with immunoprecipitated HA antibody. Wnt7a-HA interacts with COPα and COPβ2.

FIG. 20 shows immunoblot EVs secretion analysis of Wnt7a after SiRNA of COPα and COPβ2 knockdown shows disruption of Wnt7a-EVs secretion.

FIG. 21 shows immunoblot EVs secretion analysis of Wnt7a-ESP*Scramble exhibiting no impairment on Wnt7a EVs secretion after total randomization of ESP but the positively charged motifs within it (right panel).

FIG. 22 shows FoldX variation of interaction energy (AAG) for the different positively charged motifs within ESP with COPβ2, versus the crystallographic interaction determined between COPβ2 and the motif KxK, shows the strongest interaction with the KR motif. Conversely this interaction is interrupted upon single point mutation of the lysine with alanine.

FIG. 23 shows immunoblot EVs secretion analysis of Wnt7a, after punctual lysine mutation of the positively charged motifs within the ESP, shows that mutation of K247 disrupts Wnt7a-EVs secretion.

FIG. 24 shows a ribbon diagram of Wnt7a-COPβ2 interaction through the KR lysine motif within ESP.

FIG. 25 shows immunoblot EVs secretion analysis of Wnt7a after replacement of Wnt7a-ESP by either Wnt10a-ESP or Wnt16ESP containing KR and RR (right panel). Replacement with Wnt10a-ESP or Wnt16ESP rescues Wnt7a-EVs secretion.

FIG. 26 shows immunoblot EVs secretion analysis of Wnt10b after ESP removal or double arginine mutation within its ESP (right panel). Double arginine mutation disrupts Wnt10b exosomal secretion at the same extent than removal of the entire Wnt10b ESP sequence.

FIG. 27 shows ribbon diagrams of Wnt7a-COPα interaction trough the RR motif within ESP.

FIG. 28 shows iTEM of anti-HA labeling of Wnt7a-HA transfected HEK293T cells shows both types of Wnt7a secretion, on exosomes surface (arrowheads) and as free protein (arrows). Scale bar 100 nm

FIG. 29 shows relative size distribution analysis of EVs fraction from HEK293T cells.

FIG. 30 shows iTEM of anti-HA labeling of EVs from HEK293T Wnt7a-HA transfected cells, showing HA expression on EVs surface.

FIG. 31 shows immunoblot analysis of Wnt7a-EVs that are retained inside the TFF cartridge within the retentate fraction, and free-Wnt7a passes through the pores of the column and is collected in the permeate fraction. Wnt7a co-purified with EVs together with the exosomal protein CD81.

FIG. 32 shows quantification of Wnt7a expression on secreted EVs surface versus free protein secretion.

FIG. 33 shows an experimental schematic of the protocol to obtain EVs from mice hind limb muscle.

FIG. 34 shows relative size distribution analysis of EVs fraction from muscle explants.

FIG. 35 shows Immunoblot analysis of EVs fraction from muscle showing Wnt7a expression.

FIG. 36 shows hypertrophy dose-response assay of murine primary myotubes treated with muscle EVs. Data shown as fold change on myotube diameter over the control (%); Wnt7a recombinant protein was used as a positive control.

FIG. 37 shows pMHC IF representative images of hypertrophied myoblasts after muscle EVs stimulation. Scale bar 50 μm.

FIG. 38 shows IF confirmation of Wnt7a expression abrogation in Myf5^{(Cre/+)}: Wnt7a^{(fl/fl)} injured tibialis anterior at 96 h post-CTX injury. Scale bar 50 μm

FIG. 39 shows immunoblot verification of Wnt7a expression abrogation in EVs isolated from Myf5^{(Cre/+)}: Wnt7a^{(fl/fl)} hind limb muscle at 96 h post-CTX injury.

FIG. 40 shows graph display quantitative secretion analysis of each truncate from FIG. 2B. Data shown as the ratio between EVs and cells fractions. (n=3, ANOVA test p-value 2.62E-06, TUKEY test **≤0.01, ***≤0.001).

FIG. 41 shows immunoblot secretion analysis confirms secretion of the different Wnt7a truncates without the Signal Peptide.

FIG. 42 shows Wnt7a protein tertiary structure highlighting the Wnt7a minimal structure needed to be secreted.

FIG. 43 shows ΔG_{Foldx} of Wnt7a when truncating windows of 15 residues. All windows truncating the ESP are highlighted in ΔESP region. Δ1-49 and Δ301-349 don't affect folding—ΔΔG<0 respect to WT protein—and function is not lost. Δ1-212 affects protein folding. Δ251-349 don't affect folding but function is lost since a region of the ESP is truncated.

FIG. 44 shows 3D modeling of ESP insertion in a similar structural space. (Left lower) The EBP (formerly in blue) and the replaced region (AAs 172-174), the aminoacids (formerly in red) anchoring both unstructured regions. The small difference in Cα-Cα distance of residues anchoring both peptides gives room to swap them, considering as well that are in the same face of the structural surface. (Right upper) The ESP (formerly in green) modeled into the replaced region (AAs 172-174), side chains in sticks.

FIG. 45 shows HALO overexpressing cells. Representative images of HEK293T producing cells upon incubation with HALO fluorescent tag, proving the overexpression of HALO and HALO-ESP protein.

FIG. 46 shows an experimental scheme of the BirA assay protocol.

FIG. 47 shows immunoblot analysis of BioID constructs from primary myoblasts expressing myc tagged BioID2 control, BioID2-Extracellular Vesicles Signaling peptide (ESP), and Wnt7a-BioID2.

FIG. 48 shows Wnt7a-EVs secretion is regulated by interaction with Coatomer proteins. Gene Ontology (GO) term enrichment analysis for the gene set displayed in FIG. 4-A. The graph displays terms along the hierarchy within the “cellular component” branch, the analysis was performed using ClueGO plugin on Cytoscape software. The color scale shows the p-value cutoff level for each term while the circle width shows its fold enrichment. Labels of terms involving COPI vesicle localization are highlighted.

FIG. 49 shows amino acid sequence of Wnt7a wild type ESP and Wnt7a ESP Scrambled maintaining the three positively charged motifs

FIG. 50 shows structure of the assembled Coatomer complex. (Upper part) Subunits are highlighted the COPα (red) and COPβ (blue). Both subunits present an identical folding. (Lower part) Important residues on recognition of positively charged motifs are kept in sequence and structure after superimposition of both subunits.

FIG. 51 shows coatomer modeling interaction with Wnt7a, a ribbon diagram of Wnt7a-COPβ2 interaction through the KIK dilysine motif within ESP.

FIG. 52 shows coatomer modeling interaction with Wnt7a, a ribbon diagram of Wnt7a-COPβ2 interaction through the KK dilysine motif within ESP.

FIG. 53 shows alignment of Wnt family proteins showing in green the conservation degree of the KR lysine motifs among the other Wnts.

FIG. 54 shows alignment of Wnt family proteins showing in green the conservation degree of the RR among the other Wnts.

FIG. 55 shows FoldX ΔΔGs for RR and KR motifs respect to crystallographically determined interaction between COPβ2 and the motif KxK. RR modeled interaction stabilizes the interaction as similar to KR.

### DETAILED DESCRIPTION

Generally, the present disclosure is based on the surprising finding described herein that Wnt7a, and apparently other Wnt family members, are trafficked to extracellular vesicles (EVs) via interactions with coat protein complex 1 (COPI) and/or its components. COPI has not previously been associated with EVs or EV trafficking. Extracellular vesicle signal peptides (ESPs), each comprising at least one key Coatomer binding motif (CBM), are described, and these are shown to mediate EV trafficking of Wnt family members. The ESPs may be used to target other cargo polypeptides for display on EVs, thereby lending themselves to generation of recombinant EV-directed polypeptides or EVs comprising such recombinant polypeptides.

### Extracellular Vesicles

In one aspect, there is provided an extracellular vesicle (EV) comprising:
coat protein complex 1 (COPI), and
a recombinant EV-directed polypeptide comprising:
   a cargo polypeptide, and
   an extracellular vesicle signal peptide (ESP) comprising a coatomer binding motif (CBM), wherein the cargo polypeptide is tethered to an external surface of the EV via the CBM.

By “extracellular vesicle” (EV) is meant cell-derived membranous structures, including exosomes and microvesicles, and apoptotic bodies. These extracellular vesicles generally are categorized based on their size, specific markers, cellular origin and biogenesis processes. Exosomes are 40-150 nm vesicles of endosomal-origin released from the cell upon fusion of a multivesicular body (MVB) membrane with the plasma membrane. Exosomes are produced by every cell type and their release can be induced by a variety of stimuli, including stress, hypoxia, cell death, and infection. Classical microvesicles (also known as microparticles) are 100 nm-1 μm vesicles released from the cell by shedding of the plasma membrane. Cancer cells can also secrete larger microvesicles (>1 μm) called oncosomes, which only differ from classical microvesicles in regard to their size. Like exosomes, microvesicle release can be induced by stress and viral infection, and their contents are heterogeneous. Apoptotic bodies are large EVs that are released from apoptotic cells by blebbing and range in size from 200 nm to 5 μm. These phosphatidylserine- and Annexin V-coated EVs contain cytoplasmic contents from the dying cell. Traditionally, EVs that pelleted at 100,000 g were referred to as exosomes, but in fact this pellet contains a combination of microvesicles and exosomes. It is now known that separation of different types of vesicles (microvesicles, apoptotic bodies, exosomes, etc.) is possible using proper pre-clarification processes, such as Tangential Flow Filtration, used herein. Though their biogenesis pathways are distinct, exosomes and microvesicles have many similarities and are difficult to distinguish from one another once released from the cell. Recently, the International Society for Extracellular Vesicles suggested the term Small EVs (sEVs) should be used for particles less than 200 nm in size, while the term Large EVs (IEVs) should be used for particles greater than 200 nm.

In one embodiment, the EV is an exosome.

By “coat protein complex 1” or “COPI” is meant the coatomer protein complex that coats certain membrane-bound vesicles. Two types of coatomers are known. COPII is involved in anterograde transport from ER to the cis-Golgi. COPI is conventionally known to be involved in retrograde transport from trans-Golgi network to cis-Golgi network and endoplasmic reticulum. However, here it has been shown that COPI is also associated with EVs. COPI consists of seven core subunits α-COP, β′-COP, ε-COP, β-COP, δ-COP, γ-COP and ζ-COP. A cytoplasmic heptamer of these subunits, termed coatomer, is recruited to the membrane bilayer to form a COPI coat. Coatomer becomes stably membrane associated through interaction with activated Arf1. Stable association of coatomer leads to polymerization. Localized recruitment and activation of Arf1 and/or coat polymerization leads to localizes stress on the membrane, leading to vesicle scission. While COPI is known to dissociate from vesicles, residual COPI remains on the surface of vesicles.

As use herein, an “extracellular vesicle signal peptide” or “ESP” is a signal sequence containing a CBM, and which mediates EVs secretion of the cargo protein.

A “coatomer binding motif” or “CBM” as used herein, is the specific amino acid residues within and ESP that mediates interaction with COPI or one its subunits.

In one embodiment, the ESP is for binding to a α-COP (COPα or COPA), β′-COP (COP β2), or γ-COP (COPγ) subunit of the COPI. In one embodiment, the ESP is for binding to α-COP or β′-COP.

In one embodiment, the CBM comprises a two- or three-amino acid motif comprising two positively charged amino acids residues. In one embodiment, the two- or three-amino acid motif comprises KR, KK, KxK, or RR, wherein x is any amino acid. In one embodiment, the two- or three-amino acid motif comprises RR.

In one embodiment, the CBM is located in the EV-directed polypeptide: in an unstructured loop of the cargo polypeptide, in an unstructured tail that is positioned C-terminally with respect to the cargo polypeptide, or in an unstructured leader sequence that is positioned at N-terminally with respect to the cargo polypeptide, wherein the EV-directed polypeptide lacks a signal peptide.

In one embodiment, the ESP is at least 10 amino acids in length. In one embodiment, the ESP is at least 11 amino acids in length. In one embodiment, the ESP is at least 12 amino acids in length. In one embodiment, the ESP is at least 13 amino acids in length. In one embodiment, the ESP is at least 14 amino acids in length. In one embodiment, the ESP is at least 15 amino acids in length. In one embodiment, the ESP is at least 16 amino acids in length. In one embodiment, the ESP is at least 17 amino acids in length. In one embodiment, the ESP is at least 18 amino acids in length. In one embodiment, the ESP is from 18 to 34 amino acids in length.

In one embodiment, the ESP is an ESP from a protein in the Wnt family. In one embodiment, the ESP is at least 80% identical to an ESP from a protein in the Wnt family. In one embodiment, the ESP is at least 90% identical to an ESP from a protein in the Wnt family. In one embodiment, the ESP is at least 95% identical to an ESP from a protein in the Wnt family. In one embodiment, the ESP is at least 98% identical to an ESP from a protein in the Wnt family. In one embodiment, the protein in the Wnt family is human Wnt2, Wnt2b, Wnt4, Wnt5b, Wnt7a, Wnt8a, Wnt10a, Wnt10b, Wnt11, or Wnt16.

Table 1 sets forth sequence information for these Wnt family members and their respective ESPs and CBMs.

TABLE 1
Wnt Family Proteins

| | Gene | GenBank | ESP | | CBM |
|---|---|---|---|---|---|
| Name | ID | Accession | Coordinates | CBM(s) | Coordinates |
| Wnt2 | 7472 | P09544 | 245-266 | KK | 261-262 |
| Wnt2b | 7482 | Q93097 | 276-297 | RR | 292-293 |
| Wnt4 | 54361 | P56705 | 245-268 | RR | 247-248 |
| Wnt5b | 81029 | Q9H1J7 | 259-276 | RK | 259-260 |
| Wnt7a | 7476 | O00755 | 241-257 | KR | 247-248 |
| | | | | KxKK | 253-256 |
| Wnt8a | 7478 | Q9H1J5 | 218-247 | KR | 222-223 |
| Wnt10a | 80326 | Q9GZT5 | 303-332 | RR | 328-329 |
| Wnt10b | 7480 | O00744 | 286-306 | RR | 302-303 |
| Wnt11 | 7481 | O96014 | 248-271 | RK | 255-256 |
| Wnt16 | 51384 | Q9UBV4 | 261-280 | KRK | (several |
| | | | | (KR, RK) | fused) |
| | | | | RR | 268-269 |
| | | | | RK | 275-276 |

Table 2 provides sequences of the proteins listed in Table 1, with ESPs underlined and CBMs bolded.

TABLE 2
Wnt Family Protein ESPs and CMBs

| | Sequence |
|---|---|
| Name | (ESPs underlined, CBMs bolded) |
| Wnt2 | MNAPLGGIWLWLPLLLTWLTPEVNSSWWYMRATGGSSRVMCDNVPGLVSSQRQLCHRHPD |
| | VMRAISQGVAEWTAECQHQFRQHRWNCNTLDRDHSLFGRVLLRSSRESAFVYAISSAGVV |
| | FAITRACSQGEVKSCSCDPKKMGSAKDSKGIFDWGGCSDNIDYGIKFARAFVDAKERKGK |
| | DARALMNLHNNRAGRKAVKRFLKQECKCHGVSGSCTLRTCWLAMADERKTGDYLWRKYNG |
| | AIQVVMNQDGTGFTVANERFKKPTKNDLVYFENSPDYCIRDREAGSLGTAGRVCNLTSRG |
| | MDSCEVMCCGRGYDTSHVTRMTKCGCKFHWCCAVRCQDCLEALDVHTCKAPKNADWTTAT |
| Wnt2b | MLRPGGAEEAAQLPLRRASAPVPVPSPAAPDGSRASARLGLACLLLLLLLTLPARVDTSW |
| | WYIGALGARVICDNIPGLVSRQRQLCQRYPDIMRSVGEGAREWIRECQHQFRHHRWNCTT |
| | LDRDHTVFGRVMLRSSREAAFVYAISSAGVVHAITRACSQGELSVCSCDPYTRGRHHDQR |
| | GDFDWGGCSDNIHYGVRFAKAFVDAKEKRLKDARALMNLHNNRCGRTAVRRFLKLECKCH |
| | GVSGSCTLRTCWRALSDERRTGDYLRRRYDGAVQVMATQDGANFTAARQGYRRATRTDLV |
| | YFDNSPDYCVLDKAAGSLGTAGRVCSKTSKGTDGCEIMCCGRGYDTTRVTRVTQCECKFH |
| | WCCAVRCKECRNTVDVHTCKAPKKAEWLDQT |
| Wnt4 | MSPRSCLRSLRLLVFAVFSAAASNWLYLAKLSSVGSISEEETCEKLKGLIQRQVQMCKRN |
| | LEVMDSVRRGAQLAIEECQYQFRNRRWNCSTLDSLPVFGKVVTQGTREAAFVYAISSAGV |
| | AFAVTRACSSGELEKCGCDRTVHGVSPQGFQWSGCSDNIAYGVAFSQSFVDVRERSKGAS |
| | SSRALMNLHNNEAGRKAILTHMRVECKCHGVSGSCEVKTCWRAVPPFRQVGHALKEKFDG |
| | ATEVEPRRVGSSRALVPRNAQFKPHTDEDLVYLEPSPDFCEQDMRSGVLGTRGRTCNKTS |
| | KAIDGCELLCCGRGFHTAQVELAERCSCKFHWCCFVKCRQCQRLVELHTCR |
| Wnt5b | MPSLLLLFTAALLSSWAQLLTDANSWWSLALNPVQRPEMFIIGAQPVCSQLPGLSPGQRK |
| | LCQLYQEHMAYIGEGAKTGIKECQHQFRQRRWNCSTADNASVFGRVMQIGSRETAFTHAV |
| | SAAGVVNAISRACREGELSTCGCSRTARPKDLPRDWLWGGCGDNVEYGYRFAKEFVDARE |
| | REKNFAKGSEEQGRVLMNLQNNEAGRRAVYKMADVACKCHGVSGSCSLKTCWLQLAEFRK |
| | VGDRLKEKYDSAAAMRVTRKGRLELVNSRFTQPTPEDLVYVDPSPDYCLRNESTGSLGTQ |
| | GRLCNKTSEGMDGCELMCCGRGYNQFKSVQVERCHCKFHWCCFVRCKKCTEIVDQYICK |
| Wnt7a | MNRKARRCLGHLFLSLGMVYLRIGGFSSVVALGASIICNKIPGLAPRQRAICQSRPDAII |
| | VIGEGSQMGLDECQFQFRNGRWNCSALGERTVFGKELKVGSREAAFTYAIIAAGVAHAIT |
| | AACTQGNLSDCGCDKEKQGQYHRDEGWKWGGCSADIRYGIGFAKVFVDAREIKQNARTLM |
| | NLHNNEAGRKILEENMKLECKCHGVSGSCTTKTCWTTLPQFRELGYVLKDKYNEAVHVEP |
| | VRASRNKRPTFLKIKKPLSYRKPMDTDLVYIEKSPNYCEEDPVTGSVGTQGRACNKTAPQ |
| | ASGCDLMCCGRGYNTHQYARVWQCNCKFHWCCYVKCNTCSERTEMYTCK |
| Wnt8a | MGNLFMLWAALGICCAAFSASAWSVNNFLITGPKAYLTYTTSVALGAQSGIEECKFQFAW |
| | ERWNCPENALQLSTHNRLRSATRETSFIHAISSAGVMYIITKNCSMGDFENCGCDGSNNG |
| | KTGGHGWIWGGCSDNVEFGERISKLFVDSLEKGKDARALMNLHNNRAGRLAVRATMKRTC |
| | KCHGISGSCSIQTCWLQLAEFREMGDYLKAKYDQALKIEMDKRQLRAGNSAEGHWVPAEA |
| | FLPSAEAELIFLEESPDYCTCNSSLGIYGTEGRECLQNSHNTSRWERRSCGRLCTECGLQ |
| | VEERKTEVISSCNCKFQWCCTVKCDQCRHVVSKYYCARSPGSAQSLGKGSA |
| Wnt10a | MGSAHPRPWLRLRPQPQPRPALWVLLFFLLLLAAAMPRSAPNDILDLRLPPEPVLNANTV |
| | CLTLPGLSRRQMEVCVRHPDVAASAIQGIQIAIHECQHQFRDQRWNCSSLETRNKIPYES |
| | PIFSRGFRESAFAYAIAAAGVVHAVSNACALGKLKACGCDASRRGDEEAFRRKLHRLQLD |
| | ALQRGKGLSHGVPEHPALPTASPGLQDSWEWGGCSPDMGFGERFSKDFLDSREPHRDIHA |
| | RMRLHNNRVGRQAVMENMRRKCKCHGTSGSCQLKTCWQVTPEFRTVGALLRSRFHRATLI |
| | RPHNRNGGQLEPGPAGAPSPAPGAPGPRRRASPADLVYFEKSPDFCEREPRLDSAGTVGR |
| | LCNKSSAGSDGCGSMCCGRGHNILRQTRSERCHCRFHWCCFVVCEECRITEWVSVCK |
| Wnt10b | MLEEPRPRPPPSGLAGLLFLALCSRALSNEILGLKLPGEPPLTANTVCLTLSGLSKRQLG |
| | LCLRNPDVTASALQGLHIAVHECQHQLRDQRWNCSALEGGGRLPHHSAILKRGFRESAFS |
| | FSMLAAGVMHAVATACSLGKLVSCGCGWKGSGEQDRLRAKLLQLQALSRGKSFPHSLPSP |
| | GPGSSPSPGPQDTWEWGGCNHDMDFGEKFSRDFLDSREAPRDIQARMRIHNNRVGRQVVT |
| | ENLKRKCKCHGTSGSCQFKTCWRAAPEFRAVGAALRERLGRAIFIDTHNRNSGAFQPRLR |
| | PRRLSGELVYFEKSPDFCERDPTMGSPGTRGRACNKTSRLLDGCGSLCCGRGHNVLRQTR |
| | VERCHCRFHWCCYVLCDECKVTEWVNVCK |
| Wnt11 | MRARPQVCEALLFALALQTGVCYGIKWLALSKTPSALALNQTQHCKQLEGLVSAQVQLCR |
| | SNLELMHTVVHAAREVMKACRRAFADMRWNCSSIELAPNYLLDLERGTRESAFVYALSAA |
| | AISHAIARACTSGDLPGCSCGPVPGEPPGPGNRWGGCADNLSYGLLMGAKFSDAPMKVKK |
| | TGSQANKLMRLHNSEVGRQALRASLEMKCKCHGVSGSCSIRTCWKGLQELQDVAADLKTR |
| | YLSATKVVHRPMGTRKHLVPKDLDIRPVKDSELVYLQSSPDFCMKNEKVGSHGTQDRQCN |
| | KTSNGSDSCDLMCCGRGYNPYTDRVVERCHCKYHWCCYVTCRRCERTVERYVCK |
| Wnt16 | MDRAALLGLARLCALWAALLVLFPYGAQGNWMWLGIASFGVPEKLGCANLPLNSRQKELC |
| | KRKPYLLPSIREGARLGIQECGSQFRHERWNCMITAAATTAPMGASPLFGYELSSGTKET |
| | AFIYAVMAAGLVHSVTRSCSAGNMTECSCDTTLQNGGSASEGWHWGGCSDDVQYGMWFSR |
| | KFLDFPIGNTTGKENKVLLAMNLHNNEAGRQAVAKLMSVDCRCHGVSGSCAVKTCWKTMS |
| | SFEKIGHLLKDKYENSIQISDKTKRKMRRREKDQRKIPIHKDDLLYVNKSPNYCVEDKKL |
| | GIPGTQGRECNRTSEGADGCNLLCCGRGYNTHVVRHVERCECKFIWCCYVRCRRCESMTD |
| | VHTCK |

In one embodiment, the ESP comprises a sequence from a Wnt family member listed in Table 1 or 2 and comprises the respective CBM identified in Table 1 or 2. In one embodiment, the ESP comprises a sequence that is 80% identical to a sequence of a Wnt family member listed in Table 1 or 2 and comprises the respective CBM identified in Table 1 or 2. In one embodiment, the ESP comprises a sequence that is 90% identical to a sequence of a a Wnt family member listed in Table 1 or 2 and comprises the respective CBM identified in Table 1 or 2. In one embodiment, the ESP comprises a sequence that is 95% identical to a sequence of a Wnt family member listed in Table 1 or 2 and comprises the respective CBM identified in Table 1 or 2. In one embodiment, the ESP comprises a sequence that is 99% identical to a sequence of a Wnt family member listed in Table 1 or 2 and comprises the respective CBM identified in Table 1 or 2.

In one embodiment, the ESP comprises an ESP selected from the group consisting of those ESPs depicted in Table 1 and 2. In one embodiment, the ESP is 80% identical to an ESP selected from the group consisting of those ESPs depicted in Table 1 and 2 and comprises the respective CBM. In one embodiment, the ESP is 90% identical to an ESP selected from the group consisting of those ESPs depicted in Table 1 and 2 and comprises the respective CBM. In one embodiment, the ESP is 95% identical to an ESP selected from the group consisting of those ESPs depicted in Table 1 and 2 and comprises the respective CBM. In one embodiment, the ESP is 98% identical to an ESP selected from the group consisting of those ESPs depicted in Table 1 and 2 and comprises the respective CBM.

In one embodiment, the ESP is PNKKLASPRITFKPKRRV; a sequence at least 80% identical thereto that retains at least KK, KR, or RR; a sequence at least 90% identical thereto that retains at least KK, KR, or RR; or a sequence at least 95% identical thereto that retains at least KK, KR, or RR.

In one embodiment, the ESP is from a Wnt family member from a non-human species. Wnt family members in other species are identifiable, for example, by homology-based sequence searching using human Wnt family member sequences as query sequences. ESPs and CBMs can be located in non-human Wnt family members by sequence alignment. In one embodiment, the ESP is at least 80% identical to an ESP from a non-human Wnt homologue. In one embodiment, the ESP is at least 90% identical to an ESP from a non-human Wnt homologue. In one embodiment, the ESP is at least 95% identical to an ESP from a non-human Wnt homologue. The non-human Wnt homologue may be, for example, one of those Wnt7a homologues depicted in Table 3.

TABLE 3
Example Non-Human Wnt7a Family Protein ESPs and CMBs

| | Genbank | Sequence |
|---|---|---|
| Name | Accession | (ESPs underlined, CBMs bolded) |
| Mus | P24383 | MTRKARRCLGHLFLSLGIVYLRIGGFSSVVALGASIICNKIPGLAPRQRAI |
| Musculus | | CQSRPDAIIVIGEGSQMGLDECQFQFRNGRWNCSALGERTVFGKELKVGSR |
| | | EAAFTYAIIAAGVAHAITAACTQGNLSDCGCDKEKQGQYHRDEGWKWGGCS |
| | | ADIRYGIGFAKVFVDAREIKQNARTLMNLHNNEAGRKILEENMKLECKCHG |
| | | VSGSCTTKTCWTTLPQFRELGYVLKDKYNEAVHVEPVRASRNKRPTFLKIK |
| | | KPLSYRKPMDTDLVYIEKSPNYCEEDPVTGSVGTQGRACNKTAPQASGCDL |
| | | MCCGRGYNTHQYARVWQCNCKFHWCCYVKCNTCSERTEMYTCK |
| Rattus | M0R9D3 | MTRKARRCLGHLFLSLGIVYLRIGDFSSVVALGASIICNKIPGLAPRQRAI |
| norvegicus | | CQSRPDAIIVIGEGSQMGLDECQFQFRNGRWNCSALGERTVFGKELKVGSR |
| | | EAAFTYAIIAAGVAHAITAACTQGNLSDCGCDKEKQGQYHRDEGWKWGGCS |
| | | ADIRYGIGFAKVFVDAREIKQNARTLMNLHNNEAGRKILEENMKLECKCHG |
| | | VSGSCTTKTCWTTLPQFRELGYVLKDKYNEAVHVEPVRASRNKRPTFLKIK |
| | | KPLSYRKPMDTDLVYIEKSPNYCEEDPVTGSVGTQGRACNKTAPQASGCDL |
| | | MCCGRGYNTHQYARVWQCNCKFHWCCYVKCNTCSERTEMYTCK |
| Canis | J9P2Q5 | MNRKARRCLGHLFLSLGMVYLRIGGFSSVVALGASIICNKIPGLAPRQRAI |
| lupus | | CQSRPDAIIVIGEGSQMGLDECQFQFRNGRWNCSALGERTVFGKELKVGSR |
| familiaris | | EAAFTYAIIAAGVAHAITAACTQGNLSDCGCDKEKQGQYHRDEGWKWGGCS |
| | | ADIRYGIGFAKVFVDAREIKQNARTLMNLHNNEAGRKILEENMKLECKCHG |
| | | VSGSCTTKTCWTTLPQFRELGYVLKDKYNEAVHVEPVRASRNKRPTFLKIK |
| | | KPLSYRKPMDTDLVYIEKSPNYCEEDPVTGSVGTQGRACNKTAPQASGCDL |
| | | MCCGRGYNTHQYARVWQCNCKFHWCCYVKCNTCSERTEVYTCK |
| Bos | F1N6L8 | MNRKARRCLGHLFLSLGMVYLRIGGFSSVVALGASIICNKIPGLAPRQRAI |
| Taurus | | CQSRPDAIIVIGEGSQMGLDECQFQFRNGRWNCSALGERTVFGKELKVGSR |
| | | EAAFTYAIIAAGVAHAITAACTQGNLSDCGCDKEKQGQYHRDEGWKWGGCS |
| | | ADIRYGIGFAKVFVDAREIKQNARTLMNLHNNEAGRKILEENMKLECKCHG |
| | | VSGSCTTKTCWTTLPQFRELGYVLKDKYNEAVHVEPVRASRNKRPAFLKIK |
| | | KPLSYRKPMDTELVYIEKSPSYCEEDPATGSVGTQGRACNKTAPQASGCDL |
| | | MCCGRGYNTHQYARVWQCNCKFHWCCYVKCNTCSERTEVYTCK |
| Macaca | F7HEW6 | MNRKARRCLGHLFLSLGMVYLRIGGFSTVVALGASIICNKIPGLAPRQRAI |
| mulatta | | CQSRPDAIIVIGEGSQMGLDECQFQFRNGRWNCSALGERTVFGKELKVGSR |
| | | EAAFTYAIIAAGVAHAITAACTQGNLSDCGCDKEKQGQYHRDEGWKWGGCS |
| | | ADIRYGIGFAKVFVDAREIKQNARTLMNLHNNEAGRKILEENMKLECKCHG |
| | | VSGSCTTKTCWTTLPQFRELGYVLKDKYNEAVHVEPVRASRNKRPTFLKIK |
| | | KPLSYRKPMDTDLVYIEKSPNYCEEDPVTGSVGTQGRACNKTAPQASGCDL |
| | | MCCGRGYNTHQYARVWQCNCKFHWCCYVKCNTCSERTEMYTCK |
| Gallus | Q9DEB8 | MNRKTRRWIFHIFLSLGIVYIKIGGFSSVVALGASIICNKIPGLAPRQRAI |
| gallus | | CQSRPDAIIVIGEGSQMGINECQFQFRNGRWNCSALGERTVFGKELKVGSR |
| | | EAAFTYAIIAAGVAHAITAACTQGNLSDCGCDKEKQGQYHKEEGWKWGGCS |
| | | ADIRYGIGFAKVFVDAREIKQNARTLMNLHNNEAGRKILEENMKLECKCHG |
| | | VSGSCTTKTCWTTLPKFRELGYILKDKYNEAVQVEPVRASRNKRPTFLKIK |
| | | KPLSYRKPMDTDLVYIEKSPNYCEEDPVTGSVGTQGRMCNKTAQQSNGCDL |
| | | MCCGRGYNTHQYSRVWQCNCKFHWCCYVKCNTCSERTEVYTCK |
| Xenopus | F6ZXY1 | GSREAAFMYAIIAAGVAHAITTACTQGNMSDCGCDKEKQGQFHREEGWKWG |
| tropicalis | | GCSADIRYGIGFSKVFVDAREIKQNARTLMNLHNNEAGRRILKESMKSECK |
| | | CHGVSGSCTTKTCWTTLPKFRELGAILRDKYNEAIQVEPVRASRNKRPTFL |
| | | KIKNSYRKPMDTDLVYIEKSPNYCEEDPMTGSVGTQGRLCNKTAQHTSSCD |
| | | LMCCGRGYNTHQYSRVWQCNCKFHWCCYVKCNTCSERTEVFTCK |
| Danio | Q4JLT0 | MSRKTRRWIFHIFLCLGIIYLKIGGFSSVVALGASIICNKIPGLAPRQRTI |
| rerio | | CQSRPDAIIVIGEGAQMGINECQFQFKNGRWNCSALGERTVFGKELKVGSK |
| | | EAAFTYAIIAAGVAHAITAACTQGTLSGCGCDKEKQGFYNQEEGWKWGGCS |
| | | ADIRYGLSFSKVFLDAREIKQNARTLMNLHNNEVGRKILEKNMRLECKCHG |
| | | VSGSCTTKTCWTTLPKFRQLGYILKERYNHAVHVEPVRASRNKRPAFLKVK |
| | | KPYSYRKPMDTDLVYIEKSPNYCEADPVTGSMGTQGRICNKTAQHTNGCDL |
| | | MCCGRGYNTHQYSRVWQCNCKFLWCCYVKCNTCSERTEVYTCK |

In one embodiment, the ESP is for binding to y-COP.

In one embodiment, the CBM comprises FFxxBB, wherein x is any amino acid and B is a basic amino acid.

In one embodiment, the cargo protein is a therapeutic polypeptide.

By “therapeutic polypeptide” is meant any polypeptide for which delivery is desired to achieve a therapeutic end, such as disease treatment or prophylaxis.

In one embodiment the therapeutic protein comprises an antibody or an antigen-binding fragment thereof, an enzyme, a cytotoxic protein, an antigen, a receptor-binding molecule, or a protein that is deficient in disease state.

### Recombinant Polypeptides

In one aspect, there is provided a recombinant extracellular vesicle (EV)-directed polypeptide comprising:
a cargo polypeptide, and
an extracellular vesicle signal peptide (ESP) comprising a coatomer binding motif.

In one embodiment, the EV-directed polypeptide is an exosome-directed polypeptide.

In one embodiment, the ESP is for binding to a α-COP, β′-COP, or γ-COP of coat protein complex 1 (COPI).

In one embodiment, the ESP is for binding to α-COP or β′-COP.

In one embodiment, the CBM comprises a two- or three-amino acid motif comprising two positively charged amino acids residues. In one embodiment, the two- or three-amino acid motif comprises KR, KK, KxK, RK, or RR, wherein x is any amino acid. In one embodiment, the two- or three-amino acid motif comprises RR. In one embodiment, the two- or three-amino acid motif comprises KRK. In one embodiment, the two- or three-amino acid motif comprises KxK, wherein x is any amino acid. In one embodiment, the CBM comprises a four-amino acid motif comprising at least two positively charged amino acid residues. In one embodiment, the CBM comprises a four-amino acid motif comprising at least three positively charged amino acid residues. In one embodiment, the four-amino acid motif comprises KxKK.

In one embodiment, the CBM is located in the EV-directed polypeptide: in an unstructured loop of the cargo polypeptide, in an unstructured tail that is positioned C-terminally with respect to the cargo polypeptide, or in an unstructured leader sequence that is positioned at N-terminally with respect to the cargo polypeptide, wherein the EV-directed polypeptide lacks a signal peptide.

In one embodiment, the ESP is at least 10 amino acids in length. In one embodiment, the ESP is at least 11 amino acids in length. In one embodiment, the ESP is at least 12 amino acids in length. In one embodiment, the ESP is at least 13 amino acids in length. In one embodiment, the ESP is at least 14 amino acids in length. In one embodiment, the ESP is at least 15 amino acids in length. In one embodiment, the ESP is at least 16 amino acids in length. In one embodiment, the ESP is at least 17 amino acids in length. In one embodiment, the ESP is at least 18 amino acids in length. In one embodiment, the ESP is from 18 to 34 amino acids in length.

In one embodiment, the ESP is an ESP from a protein in the Wnt family. In one embodiment, the ESP is at least 80% identical to an ESP from a protein in the Wnt family. In one embodiment, the ESP is at least 90% identical to an ESP from a protein in the Wnt family. In one embodiment, the ESP is at least 95% identical to an ESP from a protein in the Wnt family. In one embodiment, the ESP is at least 98% identical to an ESP from a protein in the Wnt family. In one embodiment, the protein in the Wnt family is human Wnt2, Wnt2b, Wnt4, Wnt5b, Wnt7a, Wnt8a, Wnt10a, Wnt10b, Wnt11, or Wnt16.

In one embodiment, the ESP comprises a sequence from a Wnt family member listed in Table 1 or 2 and comprises the respective CBM identified in Table 1 or 2. In one embodiment, the ESP comprises a sequence that is 80% identical to a sequence of a Wnt family member listed in Table 1 or 2 and comprises the respective CBM identified in Table 1 or 2 In one embodiment, the ESP comprises a sequence that is 90% identical to a sequence of a a Wnt family member listed in Table 1 or 2 and comprises the respective CBM identified in Table 1 or 2. In one embodiment, the ESP comprises a sequence that is 95% identical to a sequence of a Wnt family member listed in Table 1 or 2 and comprises the respective CBM identified in Table 1 or 2. In one embodiment, the ESP comprises a sequence that is 99% identical to a sequence of a Wnt family member listed in Table 1 or 2 and comprises the respective CBM identified in Table 1 or 2.

In one embodiment, the coatomer binding motif is for binding to γ-COP.

In one embodiment, the coatomer binding motif comprises FFxxBB, wherein x is any amino acid and B is a basic amino acid.

In one embodiment, the cargo protein is a therapeutic protein.

In one embodiment, the therapeutic protein comprises an antibody or an antigen-binding fragment thereof, an enzyme, a cytotoxic protein, an antigen, a receptor-binding molecule, or a protein that is deficient in disease state.

### Nucleic Acids, Vectors, Recombinant Host Cells, and Compositions

In one aspect, there is provided a nucleic acid molecule encoding the recombinant EV-directed polypeptide as defined herein.

In one aspect, there is provided a viral particle comprising the nucleic acid as defined herein.

In one aspect, there is provided a recombinant host cell comprising the nucleic acid as defined herein.

In one aspect, a composition comprising the EV as defined herein the nucleic acid as defined herein, or the viral particle as defined herein; together with an excipient diluent, or carrier.

### Delivery Methods and Uses

In one aspect, there is provided a use of the EV as defined herein for delivery of the cargo polypeptide to a cell.

In one aspect, there is provided a use of the EV as defined herein for preparation of a composition for delivery of the cargo polypeptide to a cell.

In one aspect, there is provided the EV as defined herein for use in delivery of the cargo polypeptide to a cell.

In one aspect, there is provided a method of delivering a cargo polypeptide to a cell comprising contacting the cell with the EV as defined herein.

### Recombinant Skeletal Muscle-Directed EVs, and Delivery Methods and Uses

The skeletal muscle targeting activity of EV-bound Wnts may, in some embodiments, allow for targeting recombinant EVs comprising a payload to skeletal muscle cells.

In one aspect, there is provided a recombinant skeletal muscle-directed extracellular vesicle (EV) comprising coat protein complex 1 (COPI), a skeletal muscle targeting moiety comprising a Wnt family polypeptide, or polypeptide at least 90% identical thereto, and a payload for delivery to skeletal muscle.

The payload may be any molecule intended for delivery to skeletal muscle. The payload may be a small molecule, such as a small molecule drug, therapeutic agent, or cytotoxic agent. The payload may comprise a nucleic acid. The payload may comprise a payload polypeptide.

In one embodiment, there is provided a recombinant skeletal muscle-directed extracellular vesicle (EV) comprising coat protein complex 1 (COPI), a skeletal muscle targeting moiety comprising a Wnt family polypeptide, or polypeptide at least 90% identical thereto, and a payload polypeptide for delivery to skeletal muscle.

The “payload polypeptide” may be any molecule that it is desirably to deliver to the cells of skeletal muscle. The payload polypeptide may, for example, be an enzyme, a therapeutic polypeptide, a cytotoxic polypeptide, or a fluorescent protein.

In one embodiment, the skeletal muscle targeting moiety comprises the Wnt family member.

In one embodiment, the Wnt family member is human Wnt2, Wnt2b, Wnt4, Wnt5b, Wnt7a, Wnt8a, Wnt10a, Wnt10b, Wnt11, or Wnt16.

In one embodiment, the Wnt family member is human Wnt7a.

The polypeptide defined by percent identity to the Wnt family may be at least 80% identical to the Wnt family member. The polypeptide defined by percent identity may be at least 85% identical to the Wnt family member. The polypeptide defined by percent identity may be at least 90% identical to the Wnt family member. The polypeptide defined by percent identity may be at least 95% identical to the Wnt family member. The polypeptide defined by percent identity may be at least 98% identical to the Wnt family member. The polypeptide defined by percent identity may be at least 99% identical to the Wnt family member. In each case, alignment may be calculated across the full length of the full length sequence of the Wnt family member. The polypeptide defined by percent identity may retain substantially the same skeletal muscle targeting activity as the Wnt family member.

In one embodiment, the payload polypeptide is a free polypeptide within the EV. In these embodiments, the payload polypeptide is not linked or connected to the skeletal muscle targeting moiety.

In one embodiment, wherein the payload polypeptide is linked to the skeletal muscle targeting moiety.

In one aspect, there is provided a method for delivering a payload to skeletal muscle comprising contacting a cell with the recombinant skeletal muscle-directed EV as defined herein. The payload may be a payload polypeptide.

In one aspect, there is provided a use the recombinant skeletal muscle-directed EV as defined herein for delivery of the payload to skeletal muscle. The payload may be a payload polypeptide.

In one aspect, there is provided the recombinant skeletal muscle-directed EV as defined herein for use in delivery of the payload to skeletal muscle. The payload may be a payload polypeptide.

### Non-EV-Bound Recombinant Wnt Proteins

The identification of ESPs and CBMs within Wnts may, in some embodiments, allow recombinant Wnts to be produced that less apt to be secreted in EVs than their wild type counterparts.

In one aspect, there is provided a recombinant Wnt protein comprising an extracellular vesicle signal peptide (ESP) sequence comprising one or more coatomer binding motifs (CBMs), wherein at least one of the one or more CBMs is mutated relative to a corresponding wild-type sequence to form a mutated CBM that reduces or abrogates extracellular vesicle-targeting activity of the ESP sequence relative to the corresponding wild-type sequence.

My “mutated” is meant an amino acid sequence change relative to the same position of the corresponding wild-type sequence. Mutations may be amino acid sequences changes, deletions, insertions, or a combination thereof.

Where a “corresponding wild-type sequence” is referred to in these embodiments, it will be understood that this refers to the sequence of the parent molecule from which the recombinant Wnt protein is derived. For sample, corresponding wild-type sequences may be obtained from GenBank reference sequences. Alignments maybe generated with well-known tools.

In one embodiment, each of the one or more CBMs is mutated relative to the corresponding wild-type sequence to form mutated CBMs that reduce or abrogate extracellular vesicle-targeting activity of the ESP sequence relative to the corresponding wild-type sequence.

By “reduce” in this context is meant that the recombinant Wnt protein exhibits a reduction in secretion in EVs relative to the corresponding wild-type protein (with a corresponding increase in the fraction of free protein produced).

In one embodiment, the recombinant Wnt protein may be secreted as more than 50% free protein. In one embodiment, the recombinant Wnt protein may be secreted as more than 60% free protein. In one embodiment, the recombinant Wnt protein may be secreted as more than 70% free protein. In one embodiment, the recombinant Wnt protein may be secreted as more than 75% free protein. In one embodiment, the recombinant Wnt protein may be secreted as more than 80% free protein.

By “abrogate” or “disrupt” in this context is meant that the EV-targeting activity is substantially removed. This term must be understood in technical context, however. For example, deletion and replacement of the entirety of the ESP (see Example 2) of Wnt7a resulted in 86.7% of protein being in the free fraction, and result may vary depending on the particular Wnt.

In one embodiment, the recombinant Wnt protein may be secreted as more than 85% free protein. In one embodiment, the recombinant Wnt protein may be secreted as more than 90% free protein. In one embodiment, the recombinant Wnt protein may be secreted as more than 95% free protein.

Likewise, where it is mentioned that the recombinant Wnt is “free of EVs” it will be understood that the recombinant Wnt is secreted in a free form in a greater proportion than its corresponding wild type sequence.

In one embodiment, the mutated CBM(s) comprise(s) an amino acid substitution, deletion, and/or insertion relative to the corresponding wild-type sequence.

In one embodiment, the one or more CBMs each independently comprises a two- or three-amino acid motif comprising KR, KK, KxK, RK, or RR, wherein x is any amino acid.

In some embodiments, the mutations result a sequence change in at least one K or R in the CBM to a different amino acid. In some embodiments, the mutations result a sequence change in at least one K or R to a neutral or negatively charged amino acid. In some embodiments, more than one K and/or R residues of the CBM are mutated. It is also envisaged that the sequences could be scrambled. The sequences could be deleted and/or replaced with a non-natural sequence. Combinations of mutations are also envisaged. The effects of mutations may be tested with assays similar to those described herein.

In one embodiment, the ESP may be at least partly deleted. In one embodiment, the entirety of the ESP may be deleted. In one embodiment, the entirety of the ESP may be deleted and replaced with a different amino acid sequence. In one embodiment, the different amino acid sequence comprises a linker. In one embodiment, the linker comprises GSGS.

In the embodiments below, where the the recombinant Wnt protein is described as “comprising” a particular sequence, it will be understood that this definition accommodates the inclusion of the mutation(s) to the CBM(s) to reduce or abrogate EV-targeting activity.

In one embodiment, the recombinant Wnt protein comprises an amino acid sequence of Wnt2 (GenBank Accession No. P09544) or an amino acid sequence at least 80% identical thereto, and wherein the mutated CBM is located at amino acid positions corresponding to 261-262 of Wnt2. The amino acid sequence defined by percent identity may be at least 85% identical thereto. The amino acid sequence defined by percent identity may be at least 90% identical thereto. The amino acid sequence defined by percent identity may be at least 95% identical thereto. The amino acid sequence defined by percent identity may be at least 98% identical thereto. The amino acid sequence defined by percent identity may be at least 99% identical thereto. In one embodiment, the recombinant Wnt protein comprises an amino acid sequence of Wnt2 (GenBank Accession No. P09544). In one embodiment thereof, the ESP is deleted. In one embodiment thereof, the ESP is replaced with a linker. In one embodiment thereof, the linker comprises GSGS.

In one embodiment, the recombinant Wnt protein comprises an amino acid sequence of Wnt2b (GenBank Accession No. Q93097) or an amino acid sequence at least 80% identical thereto, and wherein the mutated CBM is located at amino acid positions corresponding to 292-293 of Wnt2b. The amino acid sequence defined by percent identity may be at least 85% identical thereto. The amino acid sequence defined by percent identity may be at least 90% identical thereto. The amino acid sequence defined by percent identity may be at least 95% identical thereto. The amino acid sequence defined by percent identity may be at least 98% identical thereto. The amino acid sequence defined by percent identity may be at least 99% identical thereto. In one embodiment, the recombinant Wnt protein comprises an amino acid sequence of Wnt2b (GenBank Accession No. Q93097). In one embodiment thereof, the ESP is deleted. In one embodiment thereof, the ESP is replaced with a linker. In one embodiment thereof, the linker comprises GSGS.

In one embodiment, the recombinant Wnt protein comprises an amino acid sequence of Wnt4 (GenBank Accession No. P56705) or an amino acid sequence at least 80% identical thereto, and wherein the mutated CBM is located at amino acid positions corresponding to 247-248 of Wnt4. The amino acid sequence defined by percent identity may be at least 85% identical thereto. The amino acid sequence defined by percent identity may be at least 90% identical thereto. The amino acid sequence defined by percent identity may be at least 95% identical thereto. The amino acid sequence defined by percent identity may be at least 98% identical thereto. The amino acid sequence defined by percent identity may be at least 99% identical thereto. In one embodiment, the recombinant Wnt protein comprises an amino acid sequence of Wnt4 (GenBank Accession No. P56705). In one embodiment thereof, the ESP is deleted. In one embodiment thereof, the ESP is replaced with a linker. In one embodiment thereof, the linker comprises GSGS.

In one embodiment, the recombinant Wnt protein comprises an amino acid sequence of Wnt5 (GenBank Accession No. 81029) or an amino acid sequence at least 80% identical thereto, and wherein the mutated CBM is located at amino acid positions corresponding to 259-260 of Wnt5. The amino acid sequence defined by percent identity may be at least 85% identical thereto. The amino acid sequence defined by percent identity may be at least 90% identical thereto. The amino acid sequence defined by percent identity may be at least 95% identical thereto. The amino acid sequence defined by percent identity may be at least 98% identical thereto. The amino acid sequence defined by percent identity may be at least 99% identical thereto. In one embodiment, the recombinant Wnt protein comprises an amino acid sequence of Wnt5 (GenBank Accession No. 81029). In one embodiment thereof, the ESP is deleted. In one embodiment thereof, the ESP is replaced with a linker. In one embodiment thereof, the linker comprises GSGS.

In one embodiment, the recombinant Wnt protein comprises an amino acid sequence of Wnt 7a (GenBank Accession No. 000755) or an amino acid sequence at least 80% identical thereto, and wherein the mutated CBM is located at amino acid positions corresponding to one or more of 247-248 and 253-256 of Wnt7a. The amino acid sequence defined by percent identity may be at least 85% identical thereto. The amino acid sequence defined by percent identity may be at least 90% identical thereto. The amino acid sequence defined by percent identity may be at least 95% identical thereto. The amino acid sequence defined by percent identity may be at least 98% identical thereto. The amino acid sequence defined by percent identity may be at least 99% identical thereto. In one embodiment, the recombinant Wnt protein comprises an amino acid sequence of Wnt 7a (GenBank Accession No. 000755). In one embodiment thereof, the ESP is deleted. In one embodiment thereof, the ESP is replaced with a linker. In one embodiment thereof, the linker comprises GSGS.

In one embodiment, the recombinant Wnt protein comprises an amino acid sequence of Wnt8a (GenBank Accession No. Q9H1J5) or an amino acid sequence at least 80% identical thereto, and wherein the mutated CBM is located at amino acid positions corresponding to 222-223 of Wnt8a. The amino acid sequence defined by percent identity may be at least 85% identical thereto. The amino acid sequence defined by percent identity may be at least 90% identical thereto. The amino acid sequence defined by percent identity may be at least 95% identical thereto. The amino acid sequence defined by percent identity may be at least 98% identical thereto. The amino acid sequence defined by percent identity may be at least 99% identical thereto. In one embodiment, the recombinant Wnt protein comprises an amino acid sequence of Wnt8a (GenBank Accession No. Q9H1J5). In one embodiment thereof, the ESP is deleted. In one embodiment thereof, the ESP is replaced with a linker. In one embodiment thereof, the linker comprises GSGS.

In one embodiment, the recombinant Wnt protein comprises an amino acid sequence of Wnt10a (GenBank Accession No. Q9GZT5) or an amino acid sequence at least 80% identical thereto, and wherein the mutated CBM is located at amino acid positions corresponding to 328-329 of Wnt10a. The amino acid sequence defined by percent identity may be at least 85% identical thereto. The amino acid sequence defined by percent identity may be at least 90% identical thereto. The amino acid sequence defined by percent identity may be at least 95% identical thereto. The amino acid sequence defined by percent identity may be at least 98% identical thereto. The amino acid sequence defined by percent identity may be at least 99% identical thereto. In one embodiment, the recombinant Wnt protein comprises an amino acid sequence of Wnt10a (GenBank Accession No. Q9GZT5). In one embodiment thereof, the ESP is deleted. In one embodiment thereof, the ESP is replaced with a linker. In one embodiment thereof, the linker comprises GSGS.

In one embodiment, the recombinant Wnt protein comprises an amino acid sequence of Wnt10b (GenBank Accession No. 000744) or an amino acid sequence at least 80% identical thereto, and wherein the mutated CBM is located at amino acid positions corresponding to 302-303 of Wnt10b. The amino acid sequence defined by percent identity may be at least 85% identical thereto. The amino acid sequence defined by percent identity may be at least 90% identical thereto. The amino acid sequence defined by percent identity may be at least 95% identical thereto. The amino acid sequence defined by percent identity may be at least 98% identical thereto. The amino acid sequence defined by percent identity may be at least 99% identical thereto. In one embodiment, the recombinant Wnt protein comprises an amino acid sequence of Wnt10b (GenBank Accession No. 000744). In one embodiment thereof, the ESP is deleted. In one embodiment thereof, the ESP is replaced with a linker. In one embodiment thereof, the linker comprises GSGS.

In one embodiment, the recombinant Wnt protein comprises an amino acid sequence of Wnt11 (GenBank Accession No. 096014) or an amino acid sequence at least 80% identical thereto, and wherein the mutated CBM is located at amino acid positions corresponding to 255-256 of Wnt11. The amino acid sequence defined by percent identity may be at least 85% identical thereto. The amino acid sequence defined by percent identity may be at least 90% identical thereto. The amino acid sequence defined by percent identity may be at least 95% identical thereto. The amino acid sequence defined by percent identity may be at least 98% identical thereto. The amino acid sequence defined by percent identity may be at least 99% identical thereto. In one embodiment, the recombinant Wnt protein comprises an amino acid sequence of Wnt11 (GenBank Accession No. 96014). In one embodiment thereof, the ESP is deleted. In one embodiment thereof, the ESP is replaced with a linker. In one embodiment thereof, the linker comprises GSGS.

In one embodiment, the recombinant Wnt protein comprises an amino acid sequence of Wnt16 (GenBank Accession No.) or an amino acid sequence at least 80% identical thereto, and wherein the mutated CBM is located at amino acid positions corresponding to one or more of 264-265, 265-266, 268-269, 269-270, and 275-276 of Wnt16. The amino acid sequence defined by percent identity may be at least 85% identical thereto. The amino acid sequence defined by percent identity may be at least 90% identical thereto. The amino acid sequence defined by percent identity may be at least 95% identical thereto. The amino acid sequence defined by percent identity may be at least 98% identical thereto. The amino acid sequence defined by percent identity may be at least 99% identical thereto. In one embodiment, the recombinant Wnt protein comprises an amino acid sequence of Wnt16 (GenBank Accession No.). In one embodiment thereof, the ESP is deleted. In one embodiment thereof, the ESP is replaced with a linker. In one embodiment thereof, the linker comprises GSGS.

In one aspect, there is provided a recombinant polypeptide comprising the recombinant Wnt protein as defined herein.

In one embodiment, there is provided a composition comprising the recombinant Wnt protein as defined herein, together with an acceptable excipient, diluent, or carrier.

In one aspect, there is provided a recombinant nucleic acid encoding the recombinant Wnt protein as defined herein.

In one embodiment, the recombinant nucleic acid comprises DNA or RNA.

In one aspect, there is provided a vector comprising the recombinant nucleic acid as defined herein.

In one aspect, there is provided a host cell comprising the recombinant nucleic acid as defined here, or the vector as defined herein.

In one aspect, there is provided a use of the recombinant nucleic acid as defined here, or the host cell defined here, for production of the recombinant Wnt protein as defined herein, wherein the recombinant Wnt protein is free of extracellular vesicles. In one embodiment, there is proportionally more recombinant Wnt protein produced as free protein compared to the corresponding wild-type sequence. Accordingly, there is proportionally less recombinant Wnt protein produced as EV-bound protein compared to the corresponding wild-type sequence.

In one aspect, there is provided a method for producing the recombinant Wnt protein as defined herein comprising introducing the recombinant nucleic acid as defined herein to a cell, and culturing the cell to produce the recombinant Wnt protein, wherein the recombinant Wnt protein is free of extracellular vesicles. In one embodiment, there is proportionally more recombinant Wnt protein produced as free protein compared to the corresponding wild-type sequence. Accordingly, there is proportionally less recombinant Wnt protein produced as EV-bound protein compared to the corresponding wild-type sequence.

In one aspect, there is provided a method for producing the recombinant Wnt protein as defined herein comprising culturing the host cell as defined herein to produce the recombinant Wnt protein, wherein the recombinant Wnt protein is free of extracellular vesicles. In one embodiment, there is proportionally more recombinant Wnt protein produced as free protein compared to the corresponding wild-type sequence. Accordingly, there is proportionally less recombinant Wnt protein produced as EV-bound protein compared to the corresponding wild-type sequence.

### EXAMPLES

### Overview

Wnt proteins are a secreted family of hydrophobic glycoproteins that regulate important developmental processes. Here the molecular mechanisms that enables long-range Wnt signaling via exosomes, a class of secreted extracellular vesicles, is investigated. It is discovered that Wnt7a is secreted at high levels on exosomes following muscle injury to stimulate regeneration. Structure-function analysis identified the signal sequence in Wnt7a, the Extracellular Vesicle Signal Peptide, which directs exosomal secretion, and revealed that palmitoylation is not required. This peptide forms a heretofore unknown functional association with Coatomer proteins through a positively charged motif to direct trafficking of Wnt to exosomes. The positively charged motif and mechanism are conserved among Wnts. These studies identify a signal peptide that traffics cargo to the surface of exosomes and elucidates the mechanism that facilitate long-range Wnt signaling. The signal peptide can be used in recombinant polypeptide constructs to target other cargo molecules to exosomes.

### Example 1

### Introduction

Exosomes are 40-150 nm small EVs of endocytic origin involved in intercellular communication that transfer bioactive cargo, for example lipids, proteins, microRNAs, and mRNAs, to distal cells. Exosomes have been used in therapeutic applications.

Wnt proteins are an evolutionary conserved family of secreted glycoproteins that govern essential developmental, growth, and regenerative processes, as well as being involved in pathological conditions like cancer. Wnt signaling plays multiple roles in regulating stem cell function, including proliferation, cell polarity and symmetric division, motility, and fate specification. Despite their relative insolubility due to the palmitoylation required for specific Frizzled receptor binding, Wnt proteins actively participate in long-range paracrine signaling between Wnt-producing cells and distal recipient cells. Several mechanisms have been proposed to mediate long-range intercellular Wnt signaling including transfer of Wnt proteins via lipoproteins, cell extensions called cytonemes, association with soluble Wnt-binding proteins, or via a class of extracellular vesicles (EVs) termed exosomes.

In vitro studies have shown different Wnt proteins are secreted on the surface of exosomes, and that exosomal-Wnts are capable of eliciting appropriate signaling in target cells. Moreover, examples have been noted where up to 40% of Wnt proteins are secreted on exosomes. Considerable in vivo evidence derived from studies in Caenorhabditis and Drosophila, support the importance of EVs for long-range Wnt signaling. To date, long-range Wnt signaling mediated by exosomes has not been documented in vivo in mammals.

Following acute injury in adult skeletal muscle, Wnt7a is highly upregulated where it positively stimulates regenerative myogenesis by acting at multiple levels. Wnt7a/Fzd7 signaling via the planar-cell-polarity pathway stimulates symmetric muscle stem cell expansion and cell motility. Wnt7a/Fzd7 signaling via the AKT/mTOR pathway in myofibers stimulates anabolic growth and hypertrophy. Consequently, intramuscular injection of Wnt7a protein significantly ameliorates disease progression in mdx mice, a mouse model for Duchenne Muscular Dystrophy (DMD). Together, these findings indicate that Wnt7a is a promising candidate therapy for DMD. However, systemic delivery of Wnt7a via the circulation has remained a challenge because of the high hydrophobicity conferred by the conserved palmitoylation.

It has been found that Wnt7a is secreted at high levels on exosomes following muscle injury. Structure function analysis was performed and a novel specific signal sequence in Wnt7a was identified that was termed the Extracellular Vesicle Signal Peptide (ESP), which comprises a positively charged motif, which mediates Wnt7a-EVs secretion. Linking of ESP sequence to other cargo resulted in secretion on EVs. Furthermore, it was found that analogous ESP sequences are found in other Wnts that are secreted on EVs. Using Bio-ID, Coatomer proteins were identified as necessary for binding the ESP to traffic Wnt7a to the exterior of EVs. Finally, modeling and mutagenesis confirmed that the interaction occurs between the positively charged motif in the ESP and COPα and COPβ2.

The Wnt family of proteins generally, and Wnt7a specifically, were selected as a model to study exosome trafficking in the hope of elucidating general principles of wider application.

### Materials & Methods

Cell culture. HEK293T cells were obtained from ATCC (CRL-3216) and verified to be free from mycoplasma contamination using the MycoSensor PCR Assay Kit (Agilent Technologies). Cells were cultured as in DMEM (Lonza) supplemented with 10% FBS, 100 U/mL penicillin, 100 U/mL streptomycin and maintained at 37° C. in a humidified incubator equilibrated with 5% CO2. Primary myoblasts were purified from C57BL/10 mice by magnetic cell separation (MACS) as previously described by Sincennes et al. Primary myoblasts were cultured on collagen-coated dishes with HAM F12-X, 10% FBS, 100 U/mL penicillin, 100 U/mL streptomycin and maintained at 37° C. in a humidified incubator equilibrated with 5% CO2. For differentiation, myoblasts were grown up to 80% confluence and growth media was replaced with differentiation medium [HAM F12-X: DMEM (1:1), 2% HS, 100 U/mL penicillin, and 100 U/mL streptomycin] for 4 days unless otherwise stated. During differentiation serums were treated to be free of extracellular vesicles prior to assays.

Mice and animal care. All experimental protocols for mice used in this study were approved in accordance with the guidelines of the Canadian Council on Animal Care. Food and water were administered ad libitum. For muscle regeneration experiments, eight week-old male mice were used, an F2 cross between the offspring of Myf5-Cre mice and Wnt7a^{fl/fl} mice in a C57BL/6 genetic background. Muscle regeneration was assessed four days following cardiotoxin injury as previously described with the following modifications. Mice were anesthetized with isoflurane and CTX injection was performed on a single injection into the TA (50 μL, 10 μM) and muscle regeneration assessed after 96 h.

Pre-embedding immunogold labeling for tissue TEM. Tibialis Anterior muscles from 8-week-old C57BL/6 mice were processed 96 h after cardiotoxin injury. Briefly, specimens were fixed in Karnovsky's fixative for 2 weeks. After fixation all segments were subsequently washed with 0.1M sodium cacodylate, 0.1% sodium borohydride, permeabilized with 0.1% triton X-100 and blocked with 10% donkey serum+0.6% fish gelatin. TA samples were incubated with Wnt7a antibody. After 48 h incubation, segments were rinsed thoroughly with PBS and incubated overnight with the secondary antibody Ultra small (0.8 nm) Gold conjugated (EMS) in blocking buffer at RT. Later, samples were rinsed with 0.1M sodium cacodylate and post-fixed with 2% glutaraldehyde in 0.1 M sodium cacodylate. Pre-embedding enhancement was realized with silver enhancement kit (AURION R-Gent SE-EM, EMS) according to the manufacturer's instructions. After enhancement, samples were secondly post-fixed with 1% osmium tetroxide in 0.1 M sodium cacodylate buffer. Then, samples were dehydrated in increasing concentration of ethanol and infiltrated in Spurr resin. Ultrathin transversal sections (80 nm) were collected onto 200-mesh copper grids and counterstained with 2% aqueous uranyl acetate and with Reynold's lead citrate. Finally, specimens were observed under a transmission electron microscope (Hitachi 7100, Gatan digital camera). For the analysis, approximately 50 immunoelectron micrographs were examined per muscle at different magnifications.

Pre-embedding immunogold labeling for cells and EVs TEM. Fixed HEK293T cells/exosome pellets were treated separately with 0.1% sodium borohydride in PBS. After a permeabilization step pellets were blocked in blocking buffer (10% donkey serum+0.6% gelatin from cold water fish skin in PBS) for 2 h. Cell/exosome pellets were incubated with the primary antibody for 48 h. Pellets were incubated overnight with the secondary antibody (Jackson ImmunoResearch). Immunogold-labelled cells and EVs were fixed with 2% glutaraldehyde in 0.1 M sodium cacodylate buffer and enhancement was performed with a silver enhancement kit on the immunogold-labelled cells. All samples were post-fixed with 1% osmium tetroxide in 0.1 M sodium cacodylate buffer. Specimens were dehydrated and embedded in resin and polymerized overnight at 70° C. Immunogold-labelled exosome ultrathin sections were observed by transmission electron microscopy at 100 000× and 150 000×.

Conditioned media production for EVs. Equal numbers of HEK293T cells were seeded and the different plasmids were transfected with linear polyethylenimine (Polysciences), accordingly to manufacturer's instructions. After 48 h of secretion in DMEM and 10% FBS exosome-depleted, conditioned media was collected for exosomal isolation. For tissue EVs a protocol has been standardized to obtained conditioned media from muscles explants. Briefly, both hind limbs of mice were injured with cardiotoxin (90 μL per leg, 10 μM). Four days later, injured muscles were harvested and cultured as explants on an exosome-depleted FBS pre-coated dish with high-glucose DMEM (Gibco) and maintained at 37° C. in a humidified incubator equilibrated with 5% CO2. After 48 h conditioned media was collected for exosomal isolation.

EVs isolation. Conditioned media (20 mL) was clarified by sequential centrifugation (300 g at 4° C. for 10 min; 2500 g at 4° C. for 10 min and 20,000 g at 4° C. for 20 min). Supernatant was transferred to Flexboy bag (Sartorius) and subjected to tangential flow filtration (TFF) under sterile conditions. Briefly, a KrosFlo Research 2i TFF system (Spectrum Laboratories) coupled to a MidGee Hoop ultrafiltration hollow fiber cartridge (GE Healthcare) 500-KDa MWCO was used. Transmembrane pressure was automatically adjusted at 3 PSI and a shear rate at 3000 s⁻¹. Sample was concentrated up to 10 mL and then subjected to continuous diafiltration. Finally, sample was concentrated at 5 mL and recovered from the cartridge. Lastly, EVs were pellet down after spinning on an ultra bench centrifuge for 30 min at 100,000 g at 4° C.

Immunoblot analysis. Immunoblot analysis was performed as described previously with the following modifications. The lysates from EVs were not clarified by centrifugation. The immunoblot transferring was performed onto PVDF membranes. All antibodies and dilutions are provided in Table 4.

TABLE 4
Antibodies and dilutions used for immunoblot analysis.

| | | | | | | | Secondary |
|---|---|---|---|---|---|---|---|
| Antibody | Application | Size | | Species | Dilution | Source | Antibody |
| Wnt7a | iTEM | | | Goat | 50 ug/mL | R&D Systems | 0.8 nm gold |
| | muscle | | | | | AF3008 | Donkey anti |
| | | | | | | | Goat. 1:50 |
| Wnt7a | iTEM Evs | | | Goat | 20 ug/mL | R&D Systems | 12 nm gold |
| | | | | | | AF3008 | Donkey anti |
| | | | | | | | Goat. 1:50 |
| HA | iTEM cells | | | Rabbit | 1:20 | Bethyl, A190- | 6 nm gold |
| | | | | | | 108A | Donkey anti |
| | | | | | | | Rabbit. 1:50 |
| pMyosin | Immuno- | | | Mouse | 1:20 | Hybridoma | Alexa Fluor |
| | fluorescence | | | | | Bank MF20 | 488 |
| | | | | | | | (Invitrogen) |
| | | | | | | | 1:1000 |
| HSP70 | Immunoblot | 70 | KDa | Rabbit | 1:1000 | SBI EXOAB- | Anti-rabbit |
| | | | | | | Hsp70A-1 | 1:5000 |
| Wnt7a | Immuno- | | | Goat | 10 ug/mL | R&D Systems | |
| | fluorescence | | | | | AF3008 | |
| CD9 | Immunoblot | 25 | KDa | Rabbit | 1:1000 | SBI EXOAB- | Anti-rabbit |
| | | | | | | CD9A-1 | 1:5000 |
| Calnexin | Immunoblot | 90 | KDa | Rabbit | 1:5000 | Abcam, | Anti-rabbit |
| | | | | | | ab22595 | 1:5000 |
| Wnt7a | Immunoblot | 39 | KDa | Goat | 1:2000 | R&D Systems | Anti-goat |
| | | | | | | AF3008 | 1:5000 |
| CD81 | Immunoblot | 18 | KDa | Rabbit | 1:500 | Saint Johns | Anti-rabbit |
| | | | | | | Lab, | 1:5000 |
| | | | | | | STJ96759 | |
| HA | Immunoblot | 1 | KDa | Rabbit | 1:1000 | Bethyl, A190- | Anti-rabbit |
| | | | | | | 108A | 1:5000 |
| HA | iTEM Evs | | | Rabbit | 1:20 | Bethyl, A190- | 12 nm gold |
| | | | | | | 108A | Donkey anti |
| | | | | | | | Rabbit. |
| | | | | | | | 1:50 |
| HALO | Immunoblot | 33 | KDa | Mouse | 1:1000 | Promega | Anti-mouse |
| | | | | | | G9211 | 1:5000 |
| HALO | Fluorescence | | | | 200 nM | Promega | |
| | | | | | | GA1110 | |
| GM130 | Proximity | | | Rabbit | 1:100 | Abcam | |
| | Ligation | | | | | ab52649 | |
| | Assay | | | | | | |
| Wnt7a | Proximity | | | Goat | 10 ug/mL | R&D Systems | |
| | Ligation | | | | | AF3008 | |
| | Assay | | | | | | |
| COPα | Proximity | | | Mouse | 1:50 | Santa Cruz | |
| | Ligation | | | | | Biotechnology | |
| | Assay | | | | | sc-398099 | |
| COPβ2 | Proximity | | | Mouse | 1:20 | Nobus NB600- | |
| | Ligation | | | | | 102 | |
| | Assay | | | | | | |
| COPα | Immunoblot | 140 | KDa | Mouse | 1:500 | Santa Cruz | Anti-mouse |
| | | | | | | Biotechnology | 1:5000 |
| | | | | | | sc-398099 | |
| COPβ2 | Immunoblot | 103 | KDa | Rabbit | 1:1000 | Cusabio | Anti-rabbit |
| | | | | | | PA529993ESR | 1:5000 |
| | | | | | | 1HU-100UL | |
| Myc | Immunoblot | 57 | KDa | Rabbit | 1:1000 | Bethyl A 190- | Anti-rabbit |
| | | | | | | 105A | 1:5000 |
| GAPDH | Immunoblot | 37 | KDa | Goat | 1:1000 | Sigma-Aldrich | Anti-goat |
| | | | | | | PLA0302- | 1:5000 |
| | | | | | | 100UL | |

Immunohistochemistry. TA muscle cryosections were rehydrated using PBS, and then fixed with 2% PFA in PBS at room temperature. After washing with PBS, permeabilization with a solution of 0.1% Triton and 0.1 M glycine in PBS was applied for 10 min at room temperature. Mouse on mouse blocking reagent was used at a dilution of 1:40 in blocking solution of 10% goat serum, 1% bovine serum albumin (BSA) and 0.1% Tween 20 in PBS for one hour at room temperature. Primary antibodies were incubated overnight. Nuclei were counterstained with DAPI before mounting in Permafluor.

Hypertrophy assay. Myoblasts were differentiated for 4 days along with EVs stimulation at 10 μg/mL (based on total extracellular vesicle protein quantification after lysis) or recombinant Wnt7a protein at 100 ng/ml. Myotubes were fixed with 4% PFA. Permeabilization and blocking solution consisting of 0.3 M glycine, 1% BSA and 0.1% Tween in PBS was added for 90 mins. p-MHC primary antibody was incubated overnight. Nuclei were counterstained with DAPI before mounting in Permafluor. FIJI software was used to analyze myotube diameter. Ten blind images were acquired per well at 20×. The 50 largest myotubes from each well were included in the analysis.

Construction of Wnt7a mutants. Wnt7a was originated from a pcDNA3-hWnt7a-HA plasmid. Wnt10a and Wnt16 originate from pcDNA-hWnt10a-V5 (Addgene 35939) or pcDNA-hWnt16-V5 (Addgene 35942) plasmid respectively. Wnt10b used herein was a gift from Marian Waterman, David Virshup and Xi He from theplasmid kit (Addgene kit #1000000022). Mutation and truncation were generated by overlap extension PCR with specially designed primers. BamHI and EcoRI restriction sites were included in primers. OE-PCR products and pcDNA3-HA vector were digested with BamHI and EcoRI and ligated with Takara ligase Solution. All constructs were verified by sequencing. All primers and coding sequences sources are provided in Table 5.

TABLE 5
Primers and coding sequences sources.

| | | Forward | Reverse | Epitope |
|---|---|---|---|---|
| Clone | Amino acid sequences | primer | primer | Tags |
| Wnt7a_ | MNRKARRCLGHLFLSLGMVYLRIGGFSSVV | gcttctcctca | aagaattc | HA |
| Δ32-212 | ATCWTTLPQFRELGYVLKDKYNEAVHVEPV | gtggtagctac | tcaagcgt | |
| | RASRNKRPTFLKIKKPLSYRKPMDTDLVYI | gtgctggacca | aatctgga | |
| | EKSPNYCEEDPVTGSVGTQGRACNKTAPQA | cactgccac | acatcgta | |
| | SGCDLMCCGRGYNTHQYARVWQCNCKFHWC | | tgggtact | |
| | CYVKCNTCSERTEMYTCKYPYDVPDYA | | tgcacgtg | |
| | | | tacatctc | |
| | | | cg | |
| Wnt7a_ | MNRKARRCLGHLFLSLGMVYLRIGGFSSVV | aaggatccacc | aagaattc | HA |
| Δ32-149 | AGGCSADIRYGIGFAKVFVDAREIKQNART | atgaaccggaa | tcaagcgt | |
| | LMNLHNNEAGRKILEENMKLECKCHGVSGS | agcgcggcgct | aatctgga | |
| | CTTKTCWTTLPQFRELGYVLKDKYNEAVHV | gcctgggccac | acatcgta | |
| | EPVRASRNKRPTFLKIKKPLSYRKPMDTDL | ctctttctcag | tgggtact | |
| | VYIEKSPNYCEEDPVTGSVGTQGRACNKTA | cctgggcatgg | tgcacgtg | |
| | PQASGCDLMCCGRGYNTHQYARVWQCNCKF | tctacctccgg | tacatctc | |
| | HWCCYVKCNTCSERTEMYTCKYPYDVPDYA | atcggtggctt | cg | |
| | | ctcctcagtgg | | |
| | | tagctggtggc | | |
| | | tgctctgccga | | |
| | | catc | | |
| Wnt7a_ | MNRKARRCLGHLFLSLGMVYLRIGGFSSVV | aaggatccacc | aagaattc | HA |
| Δ32-99 | AGSREAAFTYAIIAAGVAHAITAACTQGNL | atgaaccggaa | tcaagcgt | |
| | SDCGCDKEKQGQYHRDEGWKWGGCSADIRY | agcgcggcgct | aatctgga | |
| | GIGFAKVFVDAREIKQNARTLMNLHNNEAG | gcctgggccac | acatcgta | |
| | RKILEENMKLECKCHGVSGSCTTKTCWTTL | ctctttctcag | tgggtact | |
| | PQFRELGYVLKDKYNEAVHVEPVRASRNKR | cctgggcatgg | tgcacgtg | |
| | PTFLKIKKPLSYRKPMDTDLVYIEKSPNYC | tctacctccgg | tacatctc | |
| | EEDPVTGSVGTQGRACNKTAPQASGCDLMC | atcggtggctt | cg | |
| | CGRGYNTHQYARVWQCNCKFHWCCYVKCNT | ctcctcagtgg | | |
| | CSERTEMYTCKYPYDVPDYA | tagctgggagc | | |
| | | cgggaggctgc | | |
| | | gttc | | |
| Wnt7a_ | MNRKARRCLGHLFLSLGMVYLRIGGFSSVV | aaggatccacc | aagaattc | HA |
| Δ32-49 | AAICQSRPDAIIVIGEGSQMGLDECQFQFR | atgaaccggaa | tcaagcgt | |
| | NGRWNCSALGERTVFGKELKVGSREAAFTY | agcgcggcgct | aatctgga | |
| | AIIAAGVAHAITAACTQGNLSDCGCDKEKQ | gcctgggccac | acatcgta | |
| | GQYHRDEGWKWGGCSADIRYGIGFAKVFVD | ctctttctcag | tgggtact | |
| | AREIKQNARTLMNLHNNEAGRKILEENMKL | cctgggcatgg | tgcacgtg | |
| | ECKCHGVSGSCTTKTCWTTLPQFRELGYVL | tctacctccgg | tacatctc | |
| | KDKYNEAVHVEPVRASRNKRPTFLKIKKPL | atcggtggctt | cg | |
| | SYRKPMDTDLVYIEKSPNYCEEDPVTGSVG | ctcctcagtgg | | |
| | TQGRACNKTAPQASGCDLMCCGRGYNTHQY | tagctgcgatc | | |
| | ARVWQCNCKFHWCCYVKCNTCSERTEMYTC | tgccagagccg | | |
| | KYPYDVPDYA | gcccgac | | |
| Wnt7a_ | MNRKARRCLGHLFLSLGMVYLRIGGFSSVV | aaggatccacc | aagaattc | HA |
| Δ213-349 | ALGASIICNKIPGLAPRQRAICQSRPDAII | atgaaccggaa | tcaagcgt | |
| | VIGEGSQMGLDECQFQFRNGRWNCSALGER | agcgcggcgct | aatctgga | |
| | TVFGKELKVGSREAAFTYAIIAAGVAHAIT | g | acatcgta | |
| | AACTQGNLSDCGCDKEKQGQYHRDEGWKWG | | tgggtact | |
| | GCSADIRYGIGFAKVFVDAREIKQNARTLM | | tggtggtg | |
| | NLHNNEAGRKILEENMKLECKCHGVSGSCT | | cacgagcc | |
| | TK YPYDVPDYA | | tgac | |
| Wnt7a_ | MNRKARRCLGHLFLSLGMVYLRIGGFSSVV | aaggatccacc | aagaattc | HA |
| Δ251-349 | ALGASIICNKIPGLAPRQRAICQSRPDAII | atgaaccggaa | tcaagcgt | |
| | VIGEGSQMGLDECQFQFRNGRWNCSALGER | agcgcggcgct | aatctgga | |
| | TVFGKELKVGSREAAFTYAIIAAGVAHAIT | g | acatcgta | |
| | AACTQGNLSDCGCDKEKQGQYHRDEGWKWG | | tgggta | |
| | GCSADIRYGIGFAKVFVDAREIKQNARTLM | | ggtgggcc | |
| | NLHNNEAGRKILEENMKLECKCHGVSGSCT | | gcttgttg | |
| | TKTCWTTLPQFRELGYVLKDKYNEAVHVEP | | cggctg | |
| | VRASRNKRPYPYDVPDYA | | | |
| Wnt7a_ | MNRKARRCLGHLFLSLGMVYLRIGGFSSVV | aaggatccacc | aagaattc | HA |
| Δ301-349 | ALGASIICNKIPGLAPRQRAICQSRPDAII | atgaaccggaa | tcaagcgt | |
| | VIGEGSQMGLDECQFQFRNGRWNCSALGER | agcgcggcgct | aatctgga | |
| | TVFGKELKVGSREAAFTYAIIAAGVAHAIT | g | acatcgta | |
| | AACTQGNLSDCGCDKEKQGQYHRDEGWKWG | | tgggtact | |
| | GCSADIRYGIGFAKVFVDAREIKQNARTLM | | ggggagcc | |
| | NLHNNEAGRKILEENMKLECKCHGVSGSCT | | gtcttgtt | |
| | TKTCWTTLPQFRELGYVLKDKYNEAVHVEP | | gcag | |
| | VRASRNKRPTFLKIKKPLSYRKPMDTDLVY | | | |
| | IEKSPNYCEEDPVTGSVGTQGRACNKTAPQ | | | |
| | YPYDVPDYA | | | |
| Wnt7a_ | MNRKARRCLGHLFLSLGMVYLRIGGFSSVV | aaggatccacc | aagaattc | HA |
| Δ32-99_ | AGSREAAFTYAIIAAGVAHAITAACTQGNL | atgaaccggaa | tcaagcgt | |
| Δ301-349 | SDCGCDKEKQGQYHRDEGWKWGGCSADIRY | agcgcggcgct | aatctgga | |
| | GIGFAKVFVDAREIKQNARTLMNLHNNEAG | gcctgggccac | acatcgta | |
| | RKILEENMKLECKCHGVSGSCTTKTCWTTL | ctctttctcag | tgggtact | |
| | PQFRELGYVLKDKYNEAVHVEPVRASRNKR | cctgggcatgg | ggggagcc | |
| | PTFLKIKKPLSYRKPMDTDLVYIEKSPNYC | tctacctccgg | gtcttgtt | |
| | EEDPVTGSVGTQGRACNKTAPQ | atcggtggctt | gcag | |
| | YPYDVPDYA | ctcctcagtgg | | |
| | | tagctgggagc | | |
| | | cgggaggctgc | | |
| | | gttc | | |
| Wnt7a_ | GSREAAFTYAIIAAGVAHAITAACTQGNLS | aaggatccacc | aagaattc | HA |
| Δ1-99_ | DCGCDKEKQGQYHRDEGWKWGGCSADIRYG | atggggagccg | tcaagcgt | |
| Δ301-349 | IGFAKVFVDAREIKQNARTLMNLHNNEAGR | ggaggctgcgt | aatctgga | |
| | KILEENMKLECKCHGVSGSCTTKTCWTTLP | tc | acatcgta | |
| | QFRELGYVLKDKYNEAVHVEPVRASRNKRP | | tgggtact | |
| | TFLKIKKPLSYRKPMDTDLVYIEKSPNYCE | | ggggagcc | |
| | EDPVTGSVGTQGRACNKTAPQYPYDVPDYA | | gtcttgtt | |
| | | | gcag | |
| Wnt7a_ | MNRKARRCLGHLFLSLGMVYLRIGGFSSVV | v | gtccgtgt | HA |
| ΔEBP | ALGASIICNKIPGLAPRQRAICQSRPDAII | vcaacgaggcc | ccatgggc | |
| *GSGS | VIGEGSQMGLDECQFQFRNGRWNCSALGER | gttcacgtgga | ttgcggta | |
| | TVFGKELKVGSREAAFTYAIIAAGVAHAIT | gcct | cgacagtg | |
| | AACTQGNLSDCGCDKEKQGQYHRDEGWKWG | ggttcaggttc | aacctgaa | |
| | GCSADIRYGIGFAKVFVDAREIKQNARTLM | a | ccaggctc | |
| | NLHNNEAGRKILEENMKLECKCHGVSGSCT | ctgtcgtaccg | cacgtgaa | |
| | TKTCWTTLPQFRELGYVLKDKYNEAVHVE | caagcccatgg | cggcctcg | |
| | GSGSLSYRKPMDTDLVYIEKSPNYCEEDPV | acacggac | ttg | |
| | TGSVGTQGRACNKTAPQASGCDLMCCGRGY | | | |
| | NTHQYARVWQCNCKFHWCCYVKCNTCSERT | | | |
| | EMYTCK YPYDVPDYA | | | |
| Wnt7a_ | AICQSRPDAIIVIGEGSQMGLDECQFQFRN | aaggatccacc | aagaattc | HA |
| Δ1-49 | GRWNCSALGERTVFGKELKVGSREAAFTYA | atggcgatctg | tcaagcgt | |
| | IIAAGVAHAITAACTQGNLSDCGCDKEKQG | ccagagccggc | aatctgga | |
| | QYHRDEGWKWGGCSADIRYGIGFAKVFVDA | ccgac | acatcgta | |
| | REIKQNARTLMNLHNNEAGRKILEENMKLE | | tgggtact | |
| | CKCHGVSGSCTTKTCWTTLPQFRELGYVLK | | tgcacgtg | |
| | DKYNEAVHVEPVRASRNKRPTFLKIKKPLS | | tacatctc | |
| | YRKPMDTDLVYIEKSPNYCEEDPVTGSVGT | | cg | |
| | QGRACNKTAPQASGCDLMCCGRGYNTHQYA | | | |
| | RVWQCNCKFHWCCYVKCNTCSERTEMYTCK | | | |
| | YPYDVPDYA | | | |
| Wnt7a_ | GSREAAFTYAIIAAGVAHAITAACTQGNLS | aaggatccacc | aagaattc | HA |
| Δ1-99 | DCGCDKEKQGQYHRDEGWKWGGCSADIRYG | atggggagccg | tcaagcgt | |
| | IGFAKVFVDAREIKQNARTLMNLHNNEAGR | ggaggctgcgt | aatctgga | |
| | KILEENMKLECKCHGVSGSCTTKTCWTTLP | tc | acatcgta | |
| | QFRELGYVLKDKYNEAVHVEPVRASRNKRP | | tgggtact | |
| | TFLKIKKPLSYRKPMDTDLVYIEKSPNYCE | | tgcacgtg | |
| | EDPVTGSVGTQGRACNKTAPQASGCDLMCC | | tacatctc | |
| | GRGYNTHQYARVWQCNCKFHWCCYVKCNTC | | cg | |
| | SERTEMYTCK YPYDVPDYA | | | |
| Wnt7a_ | GGCSADIRYGIGFAKVFVDAREIKQNARTL | aaggatccacc | aagaattc | HA |
| Δ1-149 | MNLHNNEAGRKILEENMKLECKCHGVSGSC | atgggtggctg | tcaagcgt | |
| | TTKTCWTTLPQFRELGYVLKDKYNEAVHVE | ctctgccgaca | aatctgga | |
| | PVRASRNKRPTFLKIKKPLSYRKPMDTDLV | tc | acatcgta | |
| | YIEKSPNYCEEDPVTGSVGTQGRACNKTAP | | tgggtact | |
| | QASGCDLMCCGRGYNTHQYARVWQCNCKFH | | tgcacgtg | |
| | WCCYVKCNTCSERTEMYTCK YPYDVPDYA | | tacatctc | |
| | | | cg | |
| Wnt7a_ | MNRKARRCLGHLFLSLGMVYLRIGGFSSVV | caaggtctttg | gtgcaagt | HA |
| Δ3aa* | ALGASIICNKIPGLAPRQRAICQSRPDAII | tggatgcccgg | tcatgaga | |
| GSG | VIGEGSQMGLDECQFQFRNGRWNCSALGER | gagggctcggg | gtccgggc | |
| | TVFGKELKVGSREAAFTYAIIAAGVAHAIT | gaatgcccgga | attccccg | |
| | AACTQGNLSDCGCDKEKQGQYHRDEGWKWG | ctctcatgaac | agccctcc | |
| | GCSADIRYGIGFAKVFVDAREGSGNARTLM | ttgcac | cgggcatc | |
| | NLHNNEAGRKILEENMKLECKCHGVSGSCT | | cacaaaga | |
| | TKTCWTTLPQFRELGYVLKDKYNEAVHVEP | | ccttg | |
| | VRASRNKRPTFLKIKKPLSYRKPMDTDLVY | | | |
| | IEKSPNYCEEDPVTGSVGTQGRACNKTAPQ | | | |
| | ASGCDLMCCGRGYNTHQYARVWQCNCKFHW | | | |
| | CCYVKCNTCSERTEMYTCKYPYDVPDYA | | | |
| Wnt7a_ | MNRKARRCLGHLFLSLGMVYLRIGGFSSVV | tggcttcttga | cctgtgcg | HA |
| Δ3aa* | ALGASIICNKIPGLAPRQRAICQSRPDAII | tcttcaggaag | tgccagcc | |
| ESP | VIGEGSQMGLDECQFQFRNGRWNCSALGER | gtgggccgctt | gcaacaag | |
| | TVFGKELKVGSREAAFTYAIIAAGVAHAIT | gttgcggctgg | cggcccac | |
| | AACTQGNLSDCGCDKEKQGQYHRDEGWKWG | cacgcacaggc | cttcctga | |
| | GCSADIRYGIGFAKVFVDAREPVRASRNKR | tcccgggcatc | agatcaag | |
| | PTFLKIKKPNARTLMNLHNNEAGRKILEEN | cacaaagacct | aagccaaa | |
| | MKLECKCHGVSGSCTTKTCWTTLPQFRELG | tg | tgcccgga | |
| | YVLKDKYNEAVHVEPVRASRNKRPTFLKIK | | ctctcatg | |
| | KPLSYRKPMDTDLVYIEKSPNYCEEDPVTG | | aacttgc | |
| | SVGTQGRACNKTAPQASGCDLMCCGRGYNT | | | |
| | HQYARVWQCNCKFHWCCYVKCNTCSERTEM | | | |
| | YTCKYPYDVPDYA | | | |
| Wnt7a_ | MNRKARRCLGHLFLSLGMVYLRIGGFSSVV | aaggatccacc | aagaattc | HA |
| Δ213-349* | ALGASIICNKIPGLAPRQRAICQSRPDAII | atgaaccggaa | tcaagcgt | |
| ESP@172 | VIGEGSQMGLDECQFQFRNGRWNCSALGER | agcgcggcgct | aatctgga | |
| | TVFGKELKVGSREAAFTYAIIAAGVAHAIT | g | acatcgta | |
| | AACTQGNLSDCGCDKEKQGQYHRDEGWKWG | | tgggtact | |
| | GCSADIRYGIGFAKVFVDAREPVRASRNKR | | tggtggtg | |
| | PTFLKIKKPNARTLMNLHNNEAGRKILEEN | | cacgagcc | |
| | MKLECKCHGVSGSCTTK YPYDVPDYA | | tgac | |
| Wnt7a_ | MNRKARRCLGHLFLSLGMVYLRIGGFSSVV | aaggatccacc | 1- | HA |
| Δ213-349* | ALGASIICNKIPGLAPRQRAICQSRPDAII | atgaaccggaa | gcttgttg | |
| ESP | VIGEGSQMGLDECQFQFRNGRWNCSALGER | agcgcggcgct | cggctggc | |
| | TVFGKELKVGSREAAFTYAIIAAGVAHAIT | g | acgcacag | |
| | AACTQGNLSDCGCDKEKQGQYHRDEGWKWG | | gtcccgat | |
| | GCSADIRYGIGFAKVFVDAREIKQNARTLM | | cccttggt | |
| | NLHNNEAGRKILEENMKLECKCHGVSGSCT | | ggtgcacg | |
| | TKPVRASRNKRPTFLKIKKP YPYDVPDYA | | agcctgac | |
| | | | ac 2- | |
| | | | agaattct | |
| | | | caagc | |
| | | | gta atc | |
| | | | tgg aac | |
| | | | atc gta | |
| | | | tgg gta | |
| | | | tggcttct | |
| | | | tgatcttc | |
| | | | aggaaggt | |
| | | | gggccgct | |
| | | | tgttgcgg | |
| | | | ctggcacg | |
| | | | cac | |
| Wnt7a_ | PVRASRNKRPTFLKIKKPMNRKARRCLGHL | 1- | aagaattc | HA |
| *ESP | FLSLGMVYLRIGGFSSVVALGASIICNKIP | cccaccttcct | tcaagcgt | |
| Δ213-349 | GLAPRQRAICQSRPDAIIVIGEGSQMGLDE | gaagatcaaga | aatctgga | |
| | CQFQFRNGRWNCSALGERTVEGKELKVGSR | agccaggatcg | acatcgta | |
| | EAAFTYAIIAAGVAHAITAACTQGNLSDCG | ggaatgaaccg | tgggtact | |
| | CDKEKQGQYHRDEGWKWGGCSADIRYGIGF | gaaagcgcggc | tggtggtg | |
| | AKVFVDAREIKQNARTLMNLHNNEAGRKIL | gctg | cacgagcc | |
| | EENMKLECKCHGVSGSCTTK YPYDVPDYA | 2- | tgac | |
| | | aggatccccaa | | |
| | | tgcctgtgcgt | | |
| | | gccagccgcaa | | |
| | | caagcggccca | | |
| | | ccttcctgaag | | |
| | | atcaag | | |
| HALO* | MAEIGTGFPFDPHYVEVLGERMHYVDVGPR | atat | caagcggc | HA |
| ESP-HA | DGTPVLFLHGNPTSSYVWRNIIPHVAPTHR | aagctt acc | ccaccttc | |
| | CIAPDLIGMGKSDKPDLGYFFDDHVRFMDA | atg | ctgaagat | |
| | FIEALGLEEVVLVIHDWGSALGFHWAKRNP | atataagctt | caagaagc | |
| | ERVKGIAFMEFIRPIPTWDEWPEFARETFQ | atggaggatct | catac | |
| | AFRTTDVGRKLIIDQNVFIEGTLPMGVVRP | gtactttcag | cca tac | |
| | LTEVEMDHYREPFLNPVDREPLWRFPNELP | | gat gtt | |
| | IAGEPANIVALVEEYMDWLHQSPVPKLLFW | | cca gat | |
| | GTPGVLIPPAEAARLAKSLPNCKAVDIGPG | | tac gct | |
| | LNLLQEDNPDLIGSEIARWLSTLEISGGSG | | tga | |
| | PVRASRNKRPTFLKIKKPYPYDVPDYA | | gaattctt | |
| HALO* | MAEIGTGFPFDPHYVEVLGERMHYVDVGPR | atat aagctt | cttcagga | N/A |
| ESP | DGTPVLFLHGNPTSSYVWRNIIPHVAPTHR | atg atataagc | aggtgggc | |
| | CIAPDLIGMGKSDKPDLGYFFDDHVRFMDA | atggaggatctg | cgcttgtt | |
| | FIEALGLEEVVLVIHDWGSALGFHWAKRNP | ag | gcggctgg | |
| | ERVKGIAFMEFIRPIPTWDEWPEFARETFQ | | cacgcaca | |
| | AFRTTDVGRKLIIDQNVFIEGTLPMGVVRP | | ggtcccga | |
| | LTEVEMDHYREPFLNPVDREPLWRFPNELP | | tccaccgg | |
| | IAGEPANIVALVEEYMDWLHQSPVPKLLFW | | aaatctcc | |
| | GTPGVLIPPAEAARLAKSLPNCKAVDIGPG | | agagtag | |
| | LNLLQEDNPDLIGSEIARWLSTLEISGG | | | |
| | SGPVRASRNKRPTFLKIKKP | | | |
| Wnt7a- | MNRKARRCLGHLFFSLGMVYLRIGGFSSVV | 1- | 1- | MYC |
| BirA- | ALGASIICNKIPGLAPRQRAICQSRPDAII | tatagaattcg | tatagtcg | |
| myc | VIGEGSQMGLDECQFQFRNGRWNCSALGER | ccaccatgaac | accatatg | |
| | TVFGKELKVGSREAAFTYAIIAAGVAHAIT | cggaaagcgcg | tatataac | |
| | AACTQGNLSDCGCDKEKQGQYHRDEGWKWG | gcgctgcc | cggtcttg | |
| | GCSADIRYGIGFAKVFVDAREIKQNARTLM | 2- | cacgtgta | |
| | NLHNNEAGRKILEENMKLECKCHGVSGSCT | tataaccggtg | catctccg | |
| | TKTCWTTLPQFRELGYVLKDKYNEAVHVEP | gaagtggaagt | tgcgc | |
| | VRASRNKRPTFLKIKKPLSYRKPMDTDLVY | ggaagtggaag | 2- | |
| | IEKSPNYCEEDPVTGSVGTQGRACNKTAPQ | tgacttcaaga | tatacata | |
| | ASGCDLMCCGRGYNTHQYARVWQCNCKFQW | acctgatctgg | tgtcaaag | |
| | CCYVKCNKCSERTEMYTCKTGGSGSGSGSD | ctg | atcttcct | |
| | FKNLIWLKEVDSTQERLKEWNVSYGTALVA | | cggatatg | |
| | DRQTKGRGGLGRKWLSQEGGLYFSFLLNPK | | agtttctg | |
| | EFENLLQLPLVLGLSVSEALEEITEIPFSL | | ctcctcga | |
| | KWPNDVYFQEKKVSGVLCELSKDKLIVGIG | | ggcttctt | |
| | INVNQREIPEEIKDRATTLYEITGKDWDRK | | ctcaggct | |
| | EVLLKVLKRISENLKKFKEKSFKEFKGKIE | | g | |
| | SKMLYLGEEVKLLGEGKITGKLVGLSEKGG | | | |
| | ALILTEEGIKEILSGEFSLRRSLEEQKLIS | | | |
| | EEDL | | | |
| Myc- | MEQKLISEEDLDFKNLIWLKEVDSTQERLK | tatactcgagg | gtcgactc | MYC |
| BirA- | EWNVSYGTALVADRQTKGRGGLGRKWLSQE | gatcgggacct | atggcttc | |
| ESP | GGLYFSFLLNPKEFENLLQLPLVLGLSVSE | gtgcgtgccag | ttgatctt | |
| | ALEEITEIPFSLKWPNDVYFQEKKVSGVLC | ccgcaac | caggaag | |
| | ELSKDKLIVGIGINVNQREIPEEIKDRATT | | | |
| | LYEITGKDWDRKEVLLKVLKRISENLKKFK | | | |
| | EKSFKEFKGKIESKMLYLGEEVKLLGEGKI | | | |
| | TGKLVGLSEKGGALILTEEGIKEILSGEFS | | | |
| | LRRSLEGSGPVRASRNKRPTFLKIKKP | | | |
| Wnt7a_ | MNRKARRCLGHLFLSLGMVYLRIGGFSSVV | tggcgagcccg | ggtttaaa | HA |
| ESP* | ALGASIICNKIPGLAPRQRAICQSRPDAII | cgcattacctt | ggtaatgc | |
| Scramb | VIGEGSQMGLDECQFQFRNGRWNCSALGER | taaaccgaaac | gcgggctc | |
| | TVFGKELKVGSREAAFTYAIIAAGVAHAIT | gccgcgtgctg | gccagttt | |
| | AACTQGNLSDCGCDKEKQGQYHRDEGWKWG | tcgtaccgcaa | tttgttcg | |
| | GCSADIRYGIGFAKVFVDAREIKQNARTLM | gcccatg | Gctccacg | |
| | NLHNNEAGRKILEENMKLECKCHGVSGSCT | | tgaacggc | |
| | TKTCWTTLPQFRELGYVLKDKYNEAVHVEP | | ctcgttg | |
| | NKKLASPRITFKPKRRVLSYRKPMDTDLVY | | | |
| | IEKSPNYCEEDPVTGSVGTQGRACNKTAPQ | | | |
| | ASGCDLMCCGRGYNTHQYARVWQCNCKFHW | | | |
| | CCYVKCNTCSERTEMYTCKYPYDVPDYA | | | |
| Wnt7a_ | MNRKARRCLGHLFLSLGMVYLRIGGFSSVV | gtgcgtgcca | gatcttca | HA |
| K247A | ALGASIICNKIPGLAPRQRAICQSRPDAII | gccgcaacgcg | ggaaggtg | |
| | VIGEGSQMGLDECQFQFRNGRWNCSALGER | cggcccacctt | ggccgcgc | |
| | TVFGKELKVGSREAAFTYAIIAAGVAHAIT | cctgaagatc | gttgcggc | |
| | AACTQGNLSDCGCDKEKQGQYHRDEGWKWG | | tggcacgc | |
| | GCSADIRYGIGFAKVFVDAREIKQNARTLM | | ac | |
| | NLHNNEAGRKILEENMKLECKCHGVSGSCT | | | |
| | TKTCWTTLPQFRELGYVLKDKYNEAVHVEP | | | |
| | VRASRNARPTFLKIKKPLSYRKPMDTDLVY | | | |
| | IEKSPNYCEEDPVTGSVGTQGRACNKTAPQ | | | |
| | ASGCDLMCCGRGYNTHQYARVWQCNCKFHW | | | |
| | CCYVKCNTCSERTEMYTCK YPYDVPDYA | | | |
| Wnt7a_ | MNRKARRCLGHLFLSLGMVYLRIGGFSSVV | caagcggccc | gtacga | HA |
| K253A | ALGASIICNKIPGLAPRQRAICQSRPDAII | accttcctgGC | cagtggct | |
| | VIGEGSQMGLDECQFQFRNGRWNCSALGER | gatcaagaagc | tcttgatc | |
| | TVFGKELKVGSREAAFTYAIIAAGVAHAIT | cactgtcgtac | GCcaggaa | |
| | AACTQGNLSDCGCDKEKQGQYHRDEGWKWG | | ggtgggcc | |
| | GCSADIRYGIGFAKVFVDAREIKQNARTLM | | gcttg | |
| | NLHNNEAGRKILEENMKLECKCHGVSGSCT | | | |
| | TKTCWTTLPQFRELGYVLKDKYNEAVHVEP | | | |
| | VRASRNKRPTFLAIKKPLSYRKPMDTDLVY | | | |
| | IEKSPNYCEEDPVTGSVGTQGRACNKTAPQ | | | |
| | ASGCDLMCCGRGYNTHQYARVWQCNCKFHW | | | |
| | CCYVKCNTCSERTEMYTCKYPYDVPDYA | | | |
| Wnt7a_ | MNRKARRCLGHLFLSLGMVYLRIGGFSSVV | cggcccacctt | cttgcgg | HA |
| K255A | ALGASIICNKIPGLAPRQRAICQSRPDAII | cctgaagatcG | tacgacag | |
| | VIGEGSQMGLDECQFQFRNGRWNCSALGER | Cgaagccactg | tggcttcg | |
| | TVFGKELKVGSREAAFTYAIIAAGVAHAIT | tcgtaccgcaa | cgatcttc | |
| | AACTQGNLSDCGCDKEKQGQYHRDEGWKWG | g | aggaaggt | |
| | GCSADIRYGIGFAKVFVDAREIKQNARTLM | | gggccg | |
| | NLHNNEAGRKILEENMKLECKCHGVSGSCT | | | |
| | TKTCWTTLPQFRELGYVLKDKYNEAVHVEP | | | |
| | VRASRNKRPTFLKIAKPLSYRKPMDTDLVY | | | |
| | IEKSPNYCEEDPVTGSVGTQGRACNKTAPQ | | | |
| | ASGCDLMCCGRGYNTHQYARVWQCNCKFHW | | | |
| | CCYVKCNTCSERTEMYTCKYPYDVPDYA | | | |
| Wnt7a_ | MNRKARRCLGHLFLSLGMVYLRIGGFSSVV | caccttcctga | gggcttg | HA |
| K256A | ALGASIICNKIPGLAPRQRAICQSRPDAII | agatcaagGCg | cggtacga | |
| | VIGEGSQMGLDECQFQFRNGRWNCSALGER | ccactgtcgta | cagtggcg | |
| | TVFGKELKVGSREAAFTYAIIAAGVAHAIT | ccgcaagccc | ccttgatc | |
| | AACTQGNLSDCGCDKEKQGQYHRDEGWKWG | | ttcaggaa | |
| | GCSADIRYGIGFAKVFVDAREIKQNARTLM | | ggtg | |
| | NLHNNEAGRKILEENMKLECKCHGVSGSCT | | | |
| | TKTCWTTLPQFRELGYVLKDKYNEAVHVEP | | | |
| | VRASRNKRPTFLKIKAPLSYRKPMDTDLVY | | | |
| | IEKSPNYCEEDPVTGSVGTQGRACNKTAPQ | | | |
| | ASGCDLMCCGRGYNTHQYARVWQCNCKFHW | | | |
| | CCYVKCNTCSERTEMYTCKYPYDVPDYA | | | |
| Wnt7a_ | MNRKARRCLGHLFLSLGMVYLRIGGFSSVV | 1- | 1- | HA |
| ΔESP* | ALGASIICNKIPGLAPRQRAICQSRPDAII | aaggatccacc | tgggcccg | |
| Wnt10a- | VIGEGSQMGLDECQFQFRNGRWNCSALGER | atgaaccggaa | gctccagc | |
| ESP | TVFGKELKVGSREAAFTYAIIAAGVAHAIT | agcgcggcgct | tggccgcc | |
| | AACTQGNLSDCGCDKEKQGQYHRDEGWKWG | g | gttgcggt | |
| | GCSADIRYGIGFAKVFVDAREIKQNARTLM | 2-gct ccg | tgtgaggc | |
| | NLHNNEAGRKILEENMKLECKCHGVSGSCT | ggc gct ccc | tccacgtg | |
| | TKTCWTTLPQFRELGYVLKDKYNEAVHVEP | ggg ccg cgc | aacggcct | |
| | HNRNGGQLEPGPAGAPSPAPGAPGPRRRAS | cga cgg gcc | c | |
| | DTDLVYIEKSPNYCEEDPVTGSVGTQGRAC | agc | 2- | |
| | NKTAPQASGCDLMCCGRGYNTHQYARVWQC | gacacggacct | aagaattc | |
| | NCKFHWCCYVKCNTCSERTEMYTCKYPYDV | ggtgtacatc | tcaagcgt | |
| | PDYA | 3-ctg gag | aatctgga | |
| | | ccg ggc cca | acatcgta | |
| | | gcg ggg gca | tgggtact | |
| | | ccc tcg ccg | tgcacgtg | |
| | | gct ccg ggc | tacatctc | |
| | | gct ccc ggg | cg | |
| | | ccg | | |
| Wnt7a_ | MNRKARRCLGHLFLSLGMVYLRIGGFSSVV | 1- | 1- | HA |
| ΔESP* | ALGASIICNKIPGLAPRQRAICQSRPDAII | aaggatccacc | atcttttt | |
| Wnt16- | VIGEGSQMGLDECQFQFRNGRWNCSALGER | atgaaccggaa | ctctcctg | |
| ESP | TVFGKELKVGSREAAFTYAIIAAGVAHAIT | agcgcggcgct | cgcatttt | |
| | AACTQGNLSDCGCDKEKQGQYHRDEGWKWG | g2- agg aga | cctctttg | |
| | GCSADIRYGIGFAKVFVDAREIKQNARTLM | gaa aaa gat | ttttaggc | |
| | NLHNNEAGRKILEENMKLECKCHGVSGSCT | cag agg aaa | tccacgtg | |
| | TKTCWTTLPQFRELGYVLKDKYNEAVHVEP | ata cca | aacggcct | |
| | KTKRKMRRREKDQRKIPIHDTDLVYIEKSP | atc cat gac | c 2- | |
| | NYCEEDPVTGSVGTQGRACNKTAPQASGCD | acggacctggt | aagaattc | |
| | LMCCGRGYNTHQYARVWQCNCKFHWCCYVK | gtacatc | tcaagcgt | |
| | CNTCSERTEMYTCKYPYDVPDYA | | aatctgga | |
| | | | acatcgta | |
| | | | tgggtact | |
| | | | tgcacgtg | |
| | | | tacatctc | |
| | | | cg | |
| Wnt10b_ | MLEEPRPRPPPSGLAGLLFLALCSRALSNE | 1- | 1- g aga | HA |
| ΔΕSP* | ILGLKLPGEPPLTANTVCLTLSGLSKRQLG | aaggatccacc | ctt ctc | |
| GSGS | LCLRNPDVTASALQGLHIAVHECQHQLRDQ | atgctggagga | aaa gta | |
| | RWNCSALEGGGRLPHHSAILKRGFRESAFS | gccccggcc | gac cag | |
| | FSMLAAGVMHAVATACSLGKLVSCGCGWKG | 2-cgg ctg | ctctgaac | |
| | SGEQDRLRAKLLQLQALSRGKSFPHSLPSP | ggc cgg gcc | ctgaacc | |
| | GPGSSPSPGPQDTWEWGGCNHDMDFGEKES | atc ttc att | aat gaa | |
| | RDFLDSREAPRDIQARMRIHNNRVGRQVVT | ggttcaggttc | gat ggc | |
| | ENLKRKCKCHGTSGSCQFKTCWRAAPEFRA | agag ctg | ccg gcc | |
| | VGAALRERLGRAIFIGSGSELVYFEKSPDF | gtc tac ttt | cag ccg | |
| | CERDPTMGSPGTRGRACNKTSRLLDGCGSL | gag aag tct | 2- | |
| | CCGRGHNVLRQTRVERCHCRFHWCCYVLCD | c | aagaattc | |
| | ECKVTEWVNVCKYPYDVPDYA | | ctaaccgg | |
| | | | tacgcgta | |
| | | | gaatcg | |
| Wnt10b_ | MLEEPRPRPPPSGLAGLLFLALCSRALSNE | gcc ttc cag | c aaa | HA |
| RR302AA | ILGLKLPGEPPLTANTVCLTLSGLSKRQLG | ccc cgt ctg | gta gac | |
| | LCLRNPDVTASALQGLHIAVHECQHQLRDQ | cgt ccc gct | cag ctc | |
| | RWNCSALEGGGRLPHHSAILKRGFRESAFS | gcc ctc tca | tcc tga | |
| | FSMLAAGVMHAVATACSLGKLVSCGCGWKG | gga gag ctg | gag ggc | |
| | SGEQDRLRAKLLQLQALSRGKSFPHSLPSP | gtc tac ttt | agc | |
| | GPGSSPSPGPQDTWEWGGCNHDMDFGEKFS | g | gggacg | |
| | RDFLDSREAPRDIQARMRIHNNRVGRQVVT | | cag acg | |
| | ENLKRKCKCHGTSGSCQFKTCWRAAPEFRA | | ggg ctg | |
| | VGAALRERLGRAIFIDTHNRNSGAFQPRLR | | gaa ggc | |
| | PAALSGELVYFEKSPDFCERDPTMGSPGTR | | | |
| | GRACNKTSRLLDGCGSLCCGRGHNVLRQTR | | | |
| | VERCHCRFHWCCYVLCDECKVTEWVNVCKY | | | |
| | PYDVPDYA | | | |

In-silico homology modeling of Wnt7a. The homology model of human Wnt7a was constructed through its sequence annealing over the resolved structure of Wnt3 protein (PDB 6AHY) with FoldX BuildModel command (Centre for Genomic Regulation, http://foldxsuite.crg.eu/command/BuildModel). The annealing of the sequence resulted in no energetic conflicts enlighting that the folding captured by the crystal represents a stable configuration of proteins within the Wnt family. GSGS linker length was chosen in order to replace ESP. In order to affect folding, the distance criteria with respect to the terminal residues of the ESP were taken into account (afterwards confirmed experimentally, FIG. 10).

In-silico determination of the ESP region. The in-silico determination of the ESP region was performed through the free energy measurement of folding of the Wnt7a model (ΔG_{wt}) versus the free energy resulting of the truncation of windows of 15 aa (ΔG_{truncated}) along the whole sequence. Those regions not contributing to the protein folding present a very negative variation energy (ΔΔG_{truncated_WT}<<0). The N-terminal region is not structured since the mapping of the Wnt folding domain (PFAM (pfam http://pfam.xfam.org/) PF00110) starts in position 41, the C-terminal region presents also low energies being a folded region not in close contact with the rest of the protein. Besides the terminal regions, the only sequence window presenting very low energy was selected as ESP (afterwards confirmed experimentally) since it is not important for the folding, is highly variable along the Wnt family, evincing that its sequence codifies for functional behavior.

Modeling of the ESP-loop swapping. All the unstructured regions within the Wnt7a generated model that were surrounded by secondary structured regions where evaluated in terms of end-to-end distances and torsional angles to establish their ability to room the ESP region though a sequence swap. Using ModelX (Centre for Genomic Regulation http://modelx.crg.es/) fragment replacement the ESP was inserted using as anchoring terminal aminoacids GLU171 and ASN 175. Energies of the replaced model was measured then with the FoldX force field and no energetic conflicts or clashes where found, demonstrating that the sequence swapping was supported by the structure.

Uptake assays. HEK293T cells were transfected with pcDNA3_HALO and pcDNA3_HALO-EBP plasmids that were generated from a Pax7-HALO plasmid (Epoch Life Science) using PEI, as aforementioned. EVs from transfected cells were isolated as previously described and added to fresh seeded HEK293T for 15 min. After, stimulated cells were labeled with HaloTag® Ligands for Super Resolution Microscopy-Janelia 549 (Promega) accordingly to manufacturer's instructions. Cells were then fixed in 2% PFA for 5 min and washed three times with PBS. Lastly, cells were analyzed by image cytometry in the Amnis ImageStream X platform to verify the location of the fluorescence inside the cell. The flourescence detected by the Amnis ImageStream was excited using 561 nm laser and detected by the 580-30 emission filter channel.

BioID assay. Stable primary myoblast cell lines expressing BioID2 BioID2-ESP and Wnt7a-BioID2 fusion proteins were generated using the mycBioID2-pBABE-puro vector (Addgene Plasmid). Myoblasts were grown in 15 cm culture dishes at subconfluency and incubated with biotin (Sigma-Aldrich: dissolved in DMSO) at a final concentration of 50 μM for 18 h. Plates were scraped in ice cold PBS, spun at 20817 g for 5 min to concentrate cell pellet, then resuspended in RIPA lysis buffer containing protease inhibitor cocktail. Cells were incubated on ice for 30 min, and then spun down 20817 g at 4° C. for 20 min. Supernatant was transferred to new low retention Eppendorf tube, and protein concentration was quantified using Bradford reagent and spectrometry. Magnetic streptavidin beads (New England Biolabs) were used to precipitate the biotinylated protein fraction. Streptavidin beads were washed twice in RIPA lysis buffer and subsequently added to protein lysates for overnight incubation at 4° C. rotating. The following day, beads were sequentially washed with RIPA buffer, 1 M KCl, 0.1 M Na2CO3, 2 M urea in 10 mM Tris-HCl (pH 8), and a final RIPA buffer wash. Biotinylated proteins were then eluted from beads by boiling for 10 min in 25 ul 6× Laemmli buffer containing 20 mM DTT and 2 mM biotin. Supernatant was loaded into precast gradient gel (4-15% Mini-PROTEAN® TGX Stain-Free™ Protein Gel) and run for 30 min at 100V. Colloidal blue dye (Thermofisher) was applied for 3 h, then rinsed in miliQ water while shaking overnight. The entire protein containing lane for each condition was then cut out and stored in 1% acetic acid. Samples were then transferred for further processing as described below.

Proteomic analysis. Proteins were digested in-gel using trypsin (Promega) according to the method of Shevchenko. Peptide extracts were concentrated by Vacufuge (Eppendorf) and purified by ZipTip (Sigma-Millipore). LC-MS/MS was performed using a Dionex Ultimate 3000 RLSC nano HPLC (Thermo Scientific) and Orbitrap Fusion Lumos mass spectrometer (Thermo Scientific). MASCOT software version 2.6.2 (Matrix Science) was used to infer peptide and protein identities from the mass spectra. The observed spectra were matched against sequences from SwissProt (version 2020-01) and also against an in-house database of common contaminants. The results were exported to Scaffold (Proteome Software) for further validation and viewing. Enrichment heatmap was generated by computing the log 2 of the fold enrichment of each condition versus its control. Gene Ontology term enrichment analysis was performed over the “cellular component” branch using ClueGO plugin on Cytoscape software.

Proximity ligation assay (PLA). Fixed myotubes were permeabilized (0.1% Triton X-100, 0.1 M Glycine, PBS) for 10 min and blocked with Duolink Blocking Solution (Sigma) for 3 h. Incubation with primary antibodies diluted in Duolink Blocking Solution (Sigma) was performed overnight at 4° C. PLA reactions were subsequently performed using Duolink PLA probes for goat-mouse and goat-rabbit and Duolink In Situ Detection Reagents Red (Sigma) following the manufacturer's protocol. Myotubes were counterstained with GM130 to visualize the Golgi Apparatus. After the final wash, cells were mounted with VECTASHIELD Antifade Mounting Medium with DAPI (Vector Laboratories). For analysis Z-stack images of myotubes were acquired on an epifluorescent microscope equipped with a motorized stage (Zeiss AxioObserver Z1) with a step size of 0.2 μm to span the cell (25 slices in total) and images were deconvoluted using Zen Software (Zeiss). 3D sum intensity Z-projection was performed with ImageJ software.

Immunoprecipitation. Wnt7a-HA was overexpressed in HEK293T cells using Lipofectamine 2000 (Life Technologies) according to the manufacturer's instructions. Cells and EVs were isolated two days post-transfection and lysed in immunoprecipitation lysis buffer (50 mM Tris pH 7.5, 150 mM NaCl, 2 mM MgCl2, 0.5 mM EDTA, 0.5% Triton X-100, and protease inhibitors) for 30 min on ice. Lysates from cells were cleared by centrifugation and were incubated with either HA (Benthyl) or COPβ2 (Cusabio) antibodies-Dynabeads Protein G (Thermo Fisher) overnight at 4° C., accordingly to the manufacturer's instructions. Beads were washed 4 times with lysis buffer and eluted with Laemmli buffer. Immunoprecipitates were resolved by SDS-PAGE and analyzed by immunoblot with the indicated antibodies.

SIRNA silencing. siRNA transfections were performed on HEK293T cells at 16 h post-culture using Lipofectamine RNAiMAX (Life Technologies) according to the manufacturer's instructions. siRNAs for COPα and COPβ2 were purchased from Dharmacon and used at a final concentration of 10 nM and 20 nM respectively. The following day cells were transfected with Wnt7a as aforementioned.

COPα-COPβ2 binding energy determination. Departing from the determined structure of Copβ2 binding to dilysine motif KxK (PDB 4J77) other binding motifs candidates described in literature were modeled by annealing them onto the crystallized binding peptide using FoldX BuildModel command. Positively charged motifs (KR, KK, RR) demonstrated a high compatibility with the binding pocket, presenting stronger binding energies than the crystallized motifs, while the truncation of these positive residues with alanine make the interactions weaker. Results were extrapolated to Copa subunit since it presents an identical folding than the β2 subunit and a sequence conserved binding pocket (PDB 5NZR).

Statistical analysis. Experiments were performed with a minimum of three biological replicates and results are presented as the mean±SEM. Student's t-test were performed to assess the statistical significance of two-tailed analysis. For multiple comparisons ANOVA test was employed and TUKEY test for post-hoc analysis. P-values are indicated as *p≤0.05, ** p≤0.01, *** p≤0.001, and P-values <0.05 were considered to be statistically significant.

### Results

### Muscle Injury Triggers Secretion of Wnt7a on the Surface of Exosomes

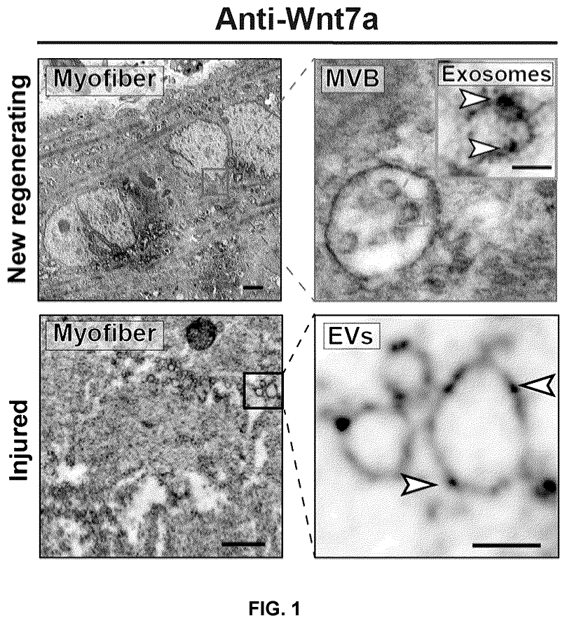

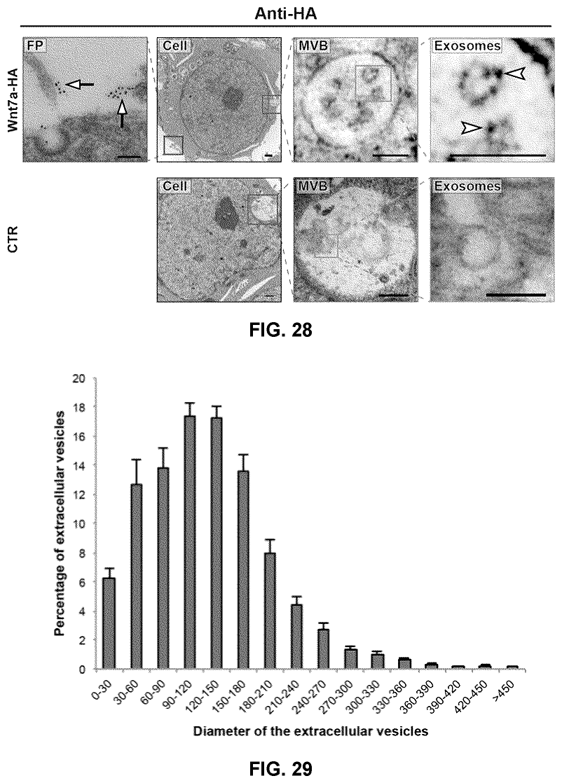

Wnt7a expression is highly upregulated in newly differentiating myofibers following acute injury of skeletal muscle. Examination of muscle cryosections 96 h following cardiotoxin injury by Immunogold Electron Transmission Microscopy (iTEM) labeling revealed extensive secretion of Wnt7a on the surface of exosomes (FIG. 1). No other types of secretion were detected such us free protein or protein aggregates. By contrast, examination of Wnt7a-HA tagged (Wnt7a-Human influenza hemagglutinin) transfected HEK293T cells by iTEM revealed that Wnt7a is secreted both as free protein and on the surface of exosomes (FIG. 28).

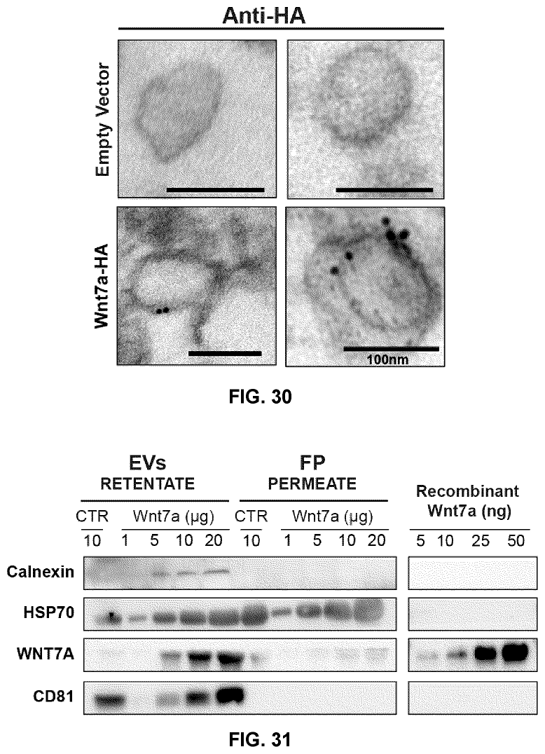

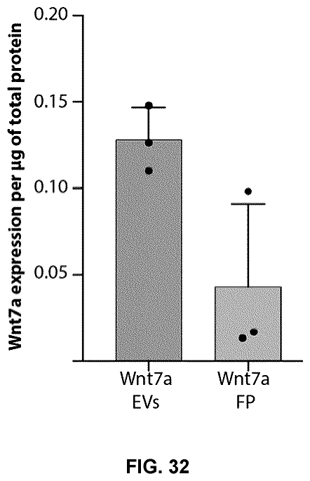

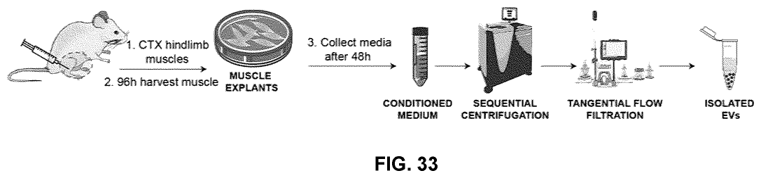

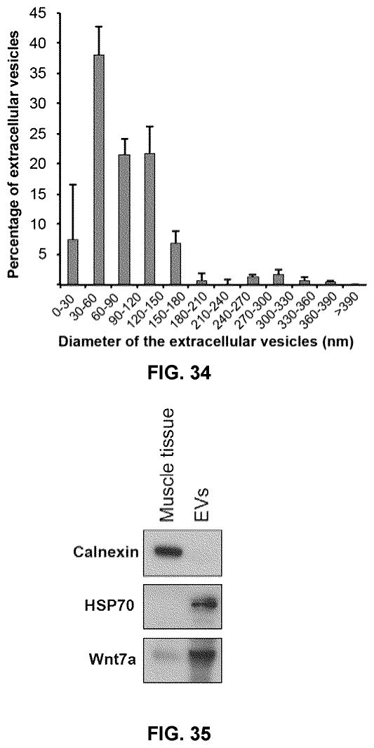

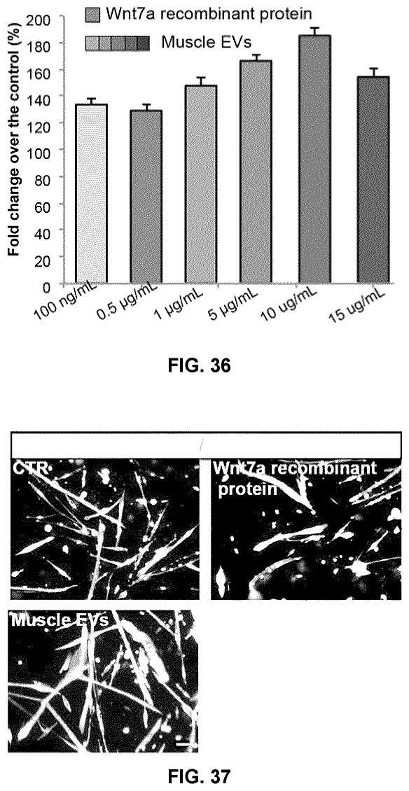

To eliminate contamination with free secreted proteins that is typically found when using ultracentrifugation to concentrate exosomal fractions, tangential flow filtration (TFF) was employed, which allows independent purification of both freely secreted Wnt7a and Wnt7a bound to EVs (see FIGS. 29-31). Quantification indicates that over 60% of secreted Wnt7a from HEK293T cells is bound to EVs (FIG. 32). Isolation of EVs from regenerating muscle using a TFF protocol (FIGS. 33-34), revealed high levels of secretion of exosomes carrying Wnt7a (FIG. 35). It has been established the capacity of Wnt7a to promote hypertrophy in vitro. Accordingly, purified exosomes from regenerating muscle induced hypertrophy of cultured myotubes indicating that Wnt7a-EVs has normal bioactivity (FIG. 36).

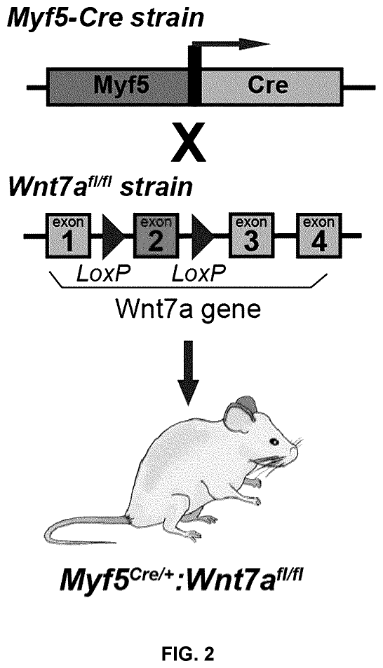

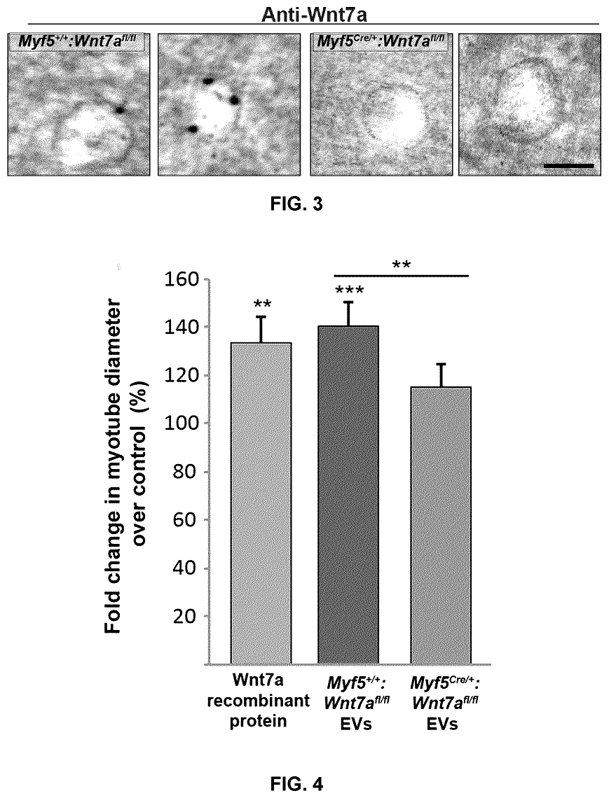

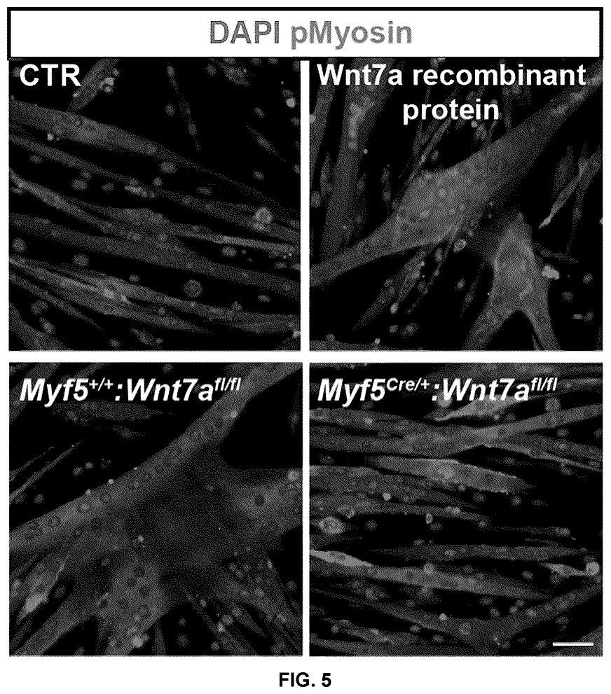

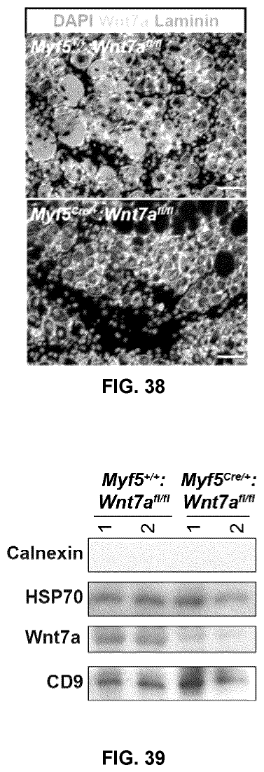

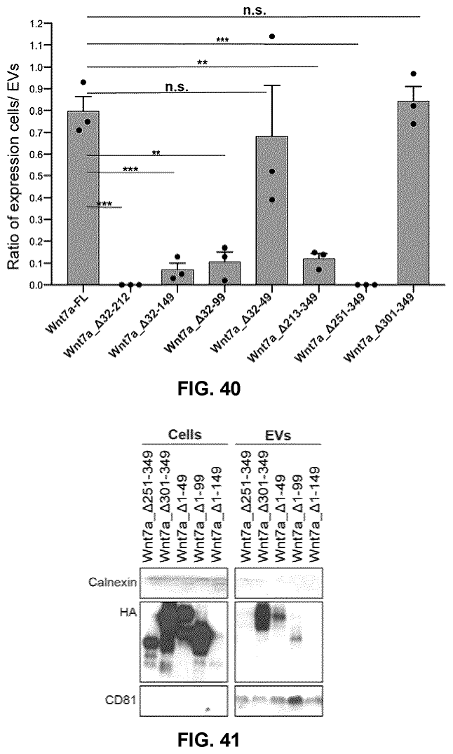

To establish whether the bioactivity of Wnt7a-EVs is due to the Wnt7a cargo, EVs were isolated from regenerating muscle from mice with a functional Wnt7a gene (Myf5^{+/+}:Wnt7a^{fl/fl}), or from mice where Wnt7a is specifically deleted in muscle (Myf5^{Cre/+}: Wnt7a^{fl/fl}) (FIG. 2), to conduct a loss of function hypertrophy study in vitro. Notably, iTEM and immunoblot analysis of EVs isolated from injured Myf5^{Cre/+}:Wnt7a^{fl/fl} muscle revealed an absence of Wnt7a, whereas EVs isolated from injured Myf5^{+/+}:Wnt7a^{fl/fl} muscle showed staining for Wnt7a (FIG. 3; FIG. 38-39). EVs (10 μg/ml) isolated from Wnt7a expressing regenerating Myf5^{+/+}:Wnt7a^{fl/fl} muscle induced a hypertrophic response in primary murine myotubes in a similar manner to recombinant Wnt7a (FIGS. 4-5). By contrast, EVs (10 μg/ml) isolated from Myf5^{Cre/+}:Wnt7a^{fl/fl} regenerating muscle lacking Wnt7a did not induce significant hypertrophy (FIGS. 4-5). Therefore, Wnt7a is bio-actively secreted in vivo from regenerating myofibers following acute injury to muscle. These data support the notion that EVs mediate long-range Wnt7a signaling during regenerative myogenesis.

### Wnt7a Secretion on EVs is Independent of Palmitoylation and Instead Requires an Internal Signal Peptide

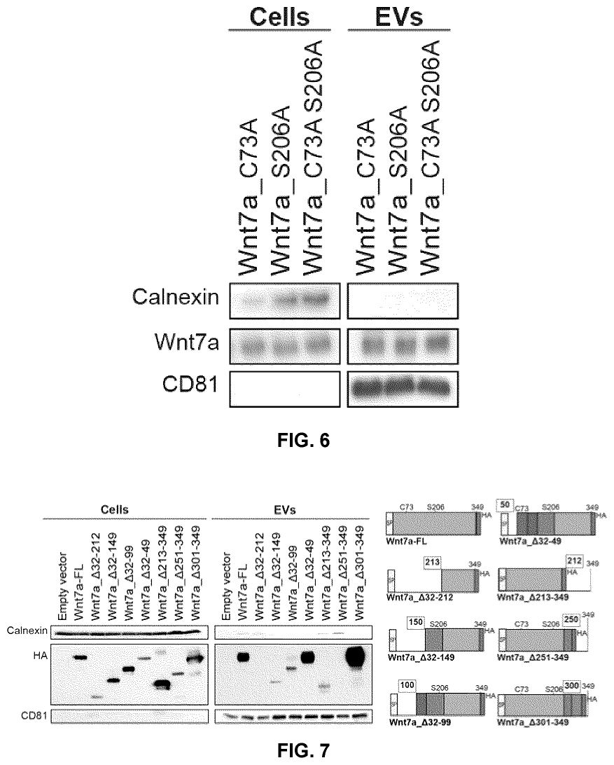

Several groups have asserted the importance of palmitoylation for Wnt secretion and activation. Therefore, Wnt7a secretion was tested following mutation of the two conserved palmitoylation sites, cysteine 73 and serine 206. These sites have been previously shown to be critical for Wnt3a secretion. Notably, it was observed that secretion of Wnt7a on EVs was entirely unaffected by mutation of the palmitoylation sites (FIG. 6).

Therefore, structure-function analysis was performed of Wnt7a to map the regions required for localization to EVs. A series of N-terminal and C-terminal deletions of Wnt7a-HA was constructed (FIG. 7). Initial N-terminal deletions were performed leaving in place the 31 aa signal peptide (SP) required for secretion of free protein. The Wnt7a variants were expressed in HEK293T cells and the amount of Wnt7a secreted on EVs was assessed by immunoblot analysis (FIG. 7).

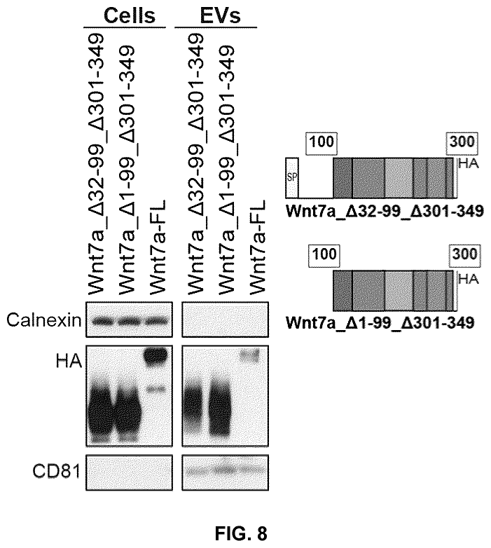

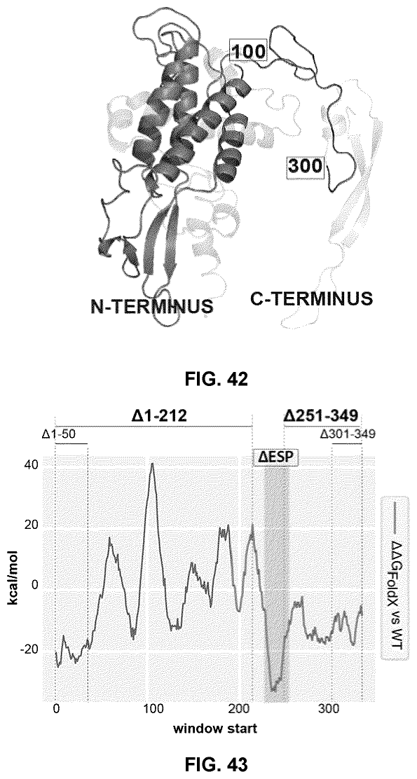

Wnt7a secretion on EVs was not impaired upon deletion of the 68aa following the SP (Wnt7a_Δ32-99), and the last 48aa (Wnt7a_Δ301-349) (FIG. 7). By contrast, deletion of additional sequences from the N-terminus (Wnt7a_Δ32-149) and C-terminus (Wnt7a_Δ251-349) appeared to abrogate secretion of EVs (FIG. 7; FIG. 41). Interestingly, it was found that deletion of the SP did not affect Wnt7a secretion on EVs on any of the constructs previously tested (FIG. 42). Indeed, Wnt7a lacking both the first 99aa and the last 48aa (Wnt7a_Δ1-99_Δ301-349), and lacking the SP, is fully secreted on EVs (FIG. 8; FIG. 43). Together, these results suggest that a region within position 100 to 300 is responsible for targeting of Wnt7a to EVs.

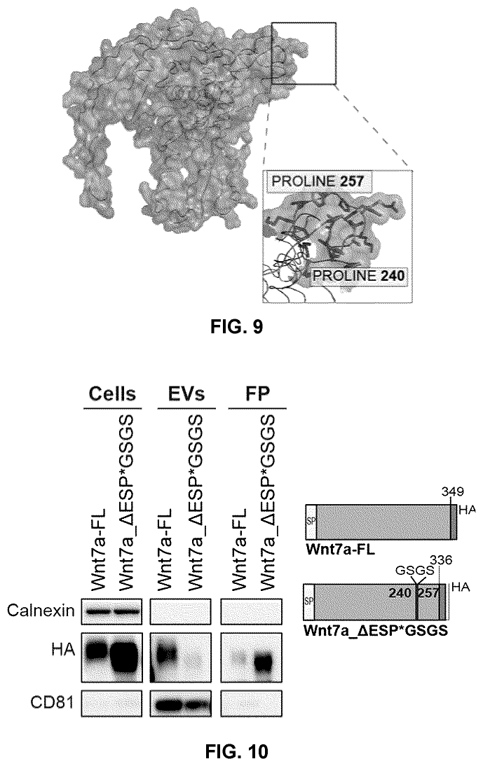

To identify the region that mediates the targeting of Wnt7a to EVs, Wnt7a was 3D-modeled based on XWnt8a structure (FIG. 9). Energetic analysis with FoldX (ΔG_{Foldx}) after truncating successive 15 aa residue regions revealed that the deletion of amino acids between positions 240 and 257 does not interfere with Wnt7a structural folding stability (FIG. 43). This low energetic region is a result of a hydrophobic random coil structure flanked by two prolines between position 240 and 257. The region was then investigated as a potential binding site that would mediate targeting of Wnt7a to EVs (FIG. 9).

Replacement of the 17 aa sequence between position 240 and 257 with the linker domain GSGS (Wnt7a_ΔESP*GSGS) resulted in a loss of Wnt7a targeting to EVs, with a corresponding increase in secretion as free protein, and with no effect on total Wnt7a protein expression (FIG. 10). This experiment suggests that the sequence PVRASRNKRPTFLKIKKP, which is herein termed the Extracellular Vesicle Signal Peptide (ESP), is responsible for targeting Wnt7a to EVs.

### The ESP Targets Proteins for Extracellular Secretion on EVs

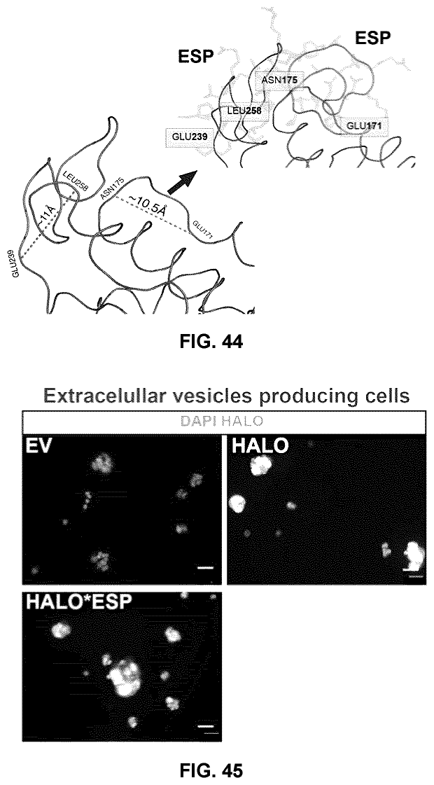

It was next investigated whether the ESP is sufficient to target a different protein for secretion on EVs. First, the ESP was added to a truncated Wnt7a that was previously found to not localize to EVs (Wnt7a_Δ213-349) (FIG. 7). A specific insertion site was chosen for the ESP within the Wnt7a_Δ213-349 truncate in order to avoid any conformational disruption or ESP offshoring. Energetic and conformational studies with FoldX showed a loop starting at position 172 as a potential insertion site for the ESP with a similar distance between loop terminals (10.89 Å versus 8.69 Å), and proximal in the 3D space to the original ESP location (FIG. 44).

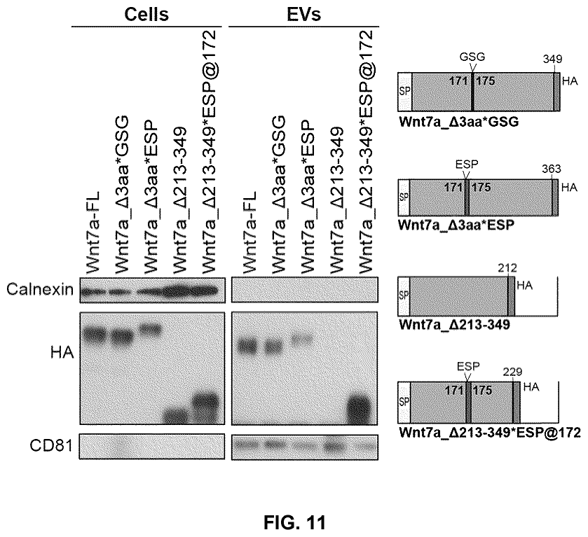

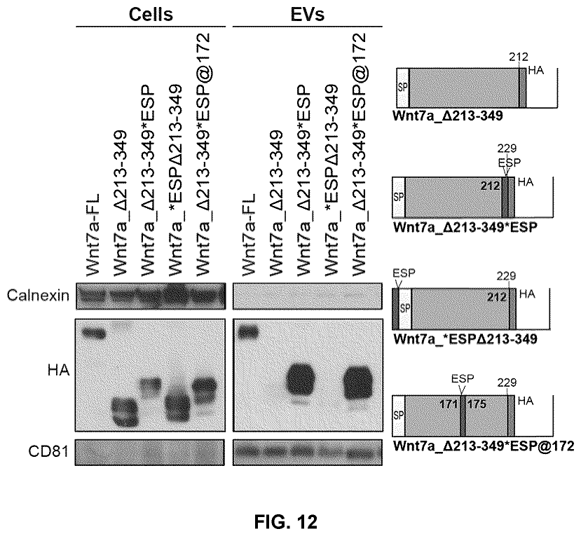

Insertion of the linker GSG or ESP between position 171 and 175 into full length Wnt7a-HA (Wnt7a-FL) had no effect on secretion on EVs (FIG. 11). Notably, insertion of the ESP between position 171 and 175 into Wnt7a_Δ213-349 (Wnt7a_Δ213-349*ESP@172) fully restored secretion on EVs (FIG. 11). Moreover, addition of ESP to the C-terminus of Wnt7a_Δ213-349 (Wnt7a_Δ213-349*ESP@212) confirmed the structurally independent capacity of ESP to target proteins for secretion on EVs (FIG. 12). However, addition of the ESP to the N-terminus of Wnt7a_Δ213-349 adjacent the SP (Wnt7a_*ESPA213-349) did not result in secretion on EVs (FIG. 12) suggesting that close proximity of both signal peptides interferes with targeting to EVs.

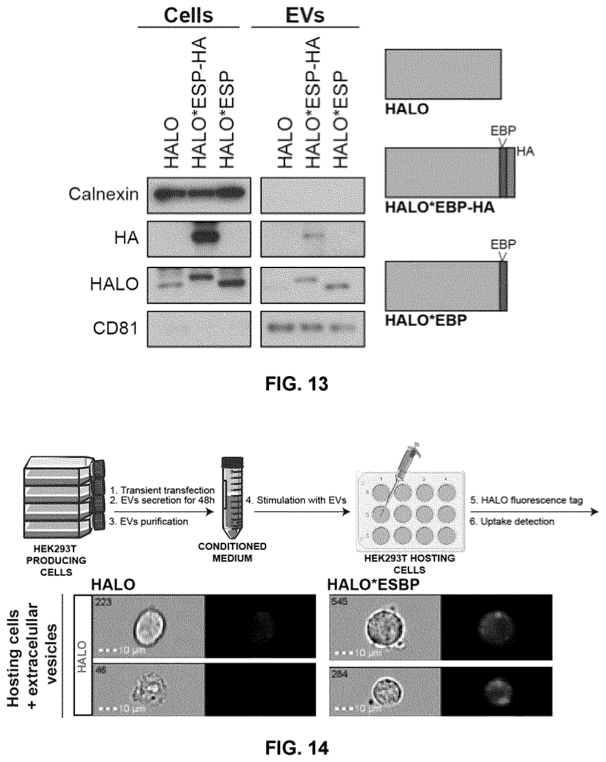

It was next contemplated whether fusing the ESP to a non-Wnt protein would confer the ability to be secreted on EVs. Therefore, the ESP was fused to the HALO tag, a 297-residue peptide derived from a bacterial enzyme designed to covalently bind to a fluorescent ligands (FIG. 13). The HALO protein was not secreted to EVs whereas HALO*ESP-HA and HALO*ESP were both efficiently secreted to EVs (FIG. 13). Furthermore, purified EVs efficiently delivered the ESP tagged HALO protein to recipient HEK293T cells, as assessed by labeling EVs with a specific fluorescent tag for HALO followed by fluorescence analysis using Amnis ImageStream cytometry (FIG. 14; FIG. 45). By contrast, EVs isolated from HALO overexpressing cells did not deliver HALO to recipient cells as revealed by the absence of fluorescence staining (FIG. 14; FIG. 45). Therefore, it was concluded that the 17 aa ESP sequence is capable of mediating targeting of proteins to EVs that can then be delivered to recipient cells.

### Secretion of Wnt7a-EVs is Regulated by Binding to the Coatomer Complex

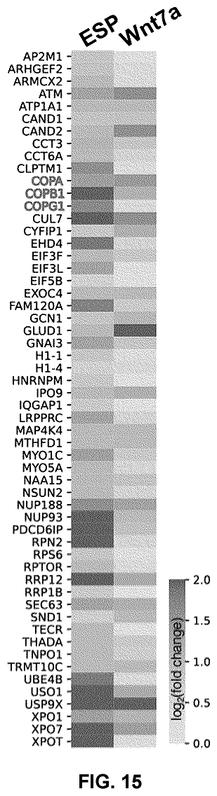

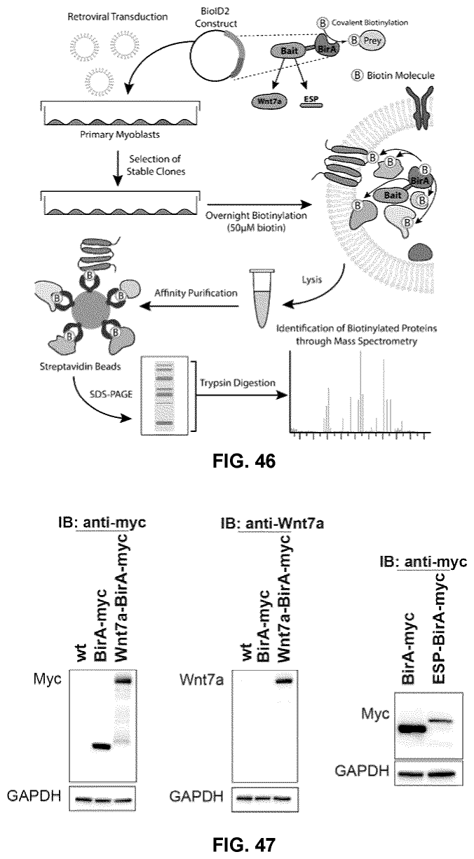

To investigate the molecular basis whereby the ESP mediates the secretion of Wnt7a on EVs, BioID analysis was performed to identify potential binding proteins. Myc-tagged BirA was used—a highly efficient proximity dependent biotin ligase—that tags proteins interacting with the constructs, even if the interaction is transient (FIG. 46). Specifically, mouse primary myoblasts were generated that express Wnt7a-BirA, ESP-BirA, or unmodified BirA as a control (FIG. 47). Mass spectrometry identification of biotinylated proteins isolated from transfected cells revealed that Coatomer proteins were among the most enriched candidates within the ESP-BirA protein-interactome (FIG. 15). Moreover, coatomer proteins were similarly present within the Wnt7a-BirA interactome (FIG. 15).

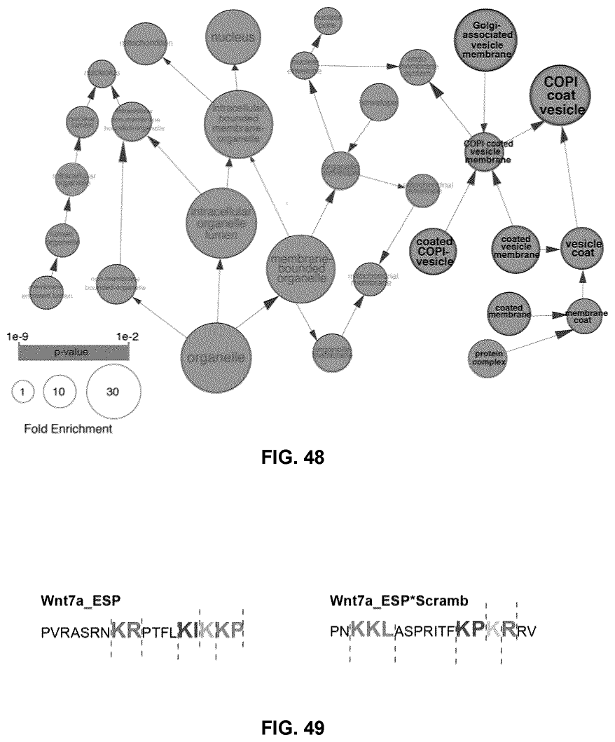

GO-term analysis of the common ESP and Wnt7a interacting proteins strongly supports the hypothesis that Wnt7a is secreted via the COPI vesicle pathway (FIG. 48). This was surprising because COPI has not apparently been previously linked to exosome trafficking. COPI coated vesicle related terms exhibit a clear localization signature for proteins presenting an increase of 50% (log 2(FC)>0.5849) on ESP condition and any increase (log 2(FC)>0) on Wnt7a condition are evaluated. COPI vesicles are protein-coated vesicular carriers that, according to conventional knowledge, mediate the retrograde transport from the Endoplasmatic Reticulum to the Golgi Apparatus (ER-GA), and within the Golgi apparatus. COPI vesicles consists of a heptamere, termed Coatomer, that are recruited together along with the GTPase ARF1 to curve the membrane bilayer to form the COPI vesicle and mediate intracellular protein transport. Coatomer is formed of seven core subunits: COPα, COPβ2, COPε, COPβ, COPδ, COPγ and COPζ, with COPα, COPβ2, and COPε involved in binding to protein cargo. The remaining coatomer subunits correspond to adaptin subunits.

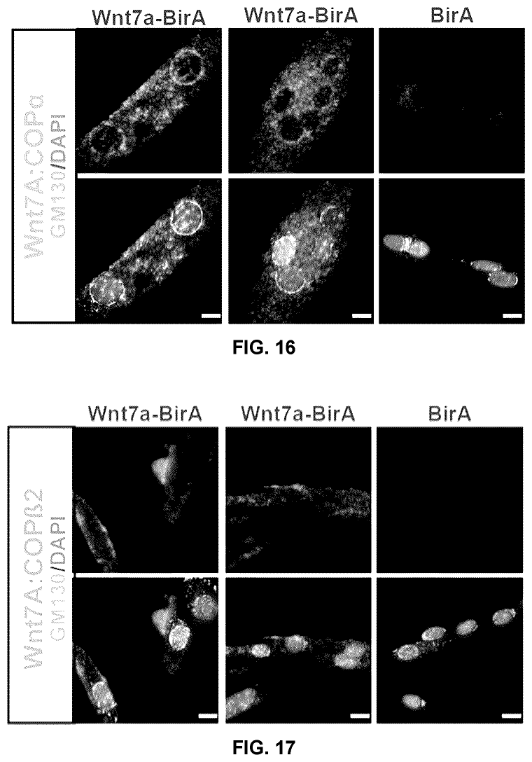

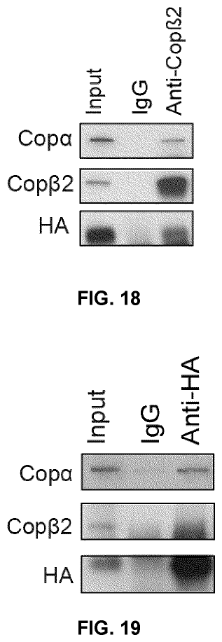

Proximity Ligation Assays between Wnt7a and COPα or COPβ2 confirmed the interaction of Wnt7a with these coatomers, displaying a different pattern of interaction at the Golgi versus the cell membrane respectively (FIGS. 16-17). These findings were corroborated by immunoprecipitation of COPβ2 in Wnt7a-HA transfected HEK293T cells, where Wnt7a-HA was found to interact with COPα and COPβ2 (FIG. 18). The same results were obtained with the reciprocal immunoprecipitation of Wnt7a-HA (FIG. 19).

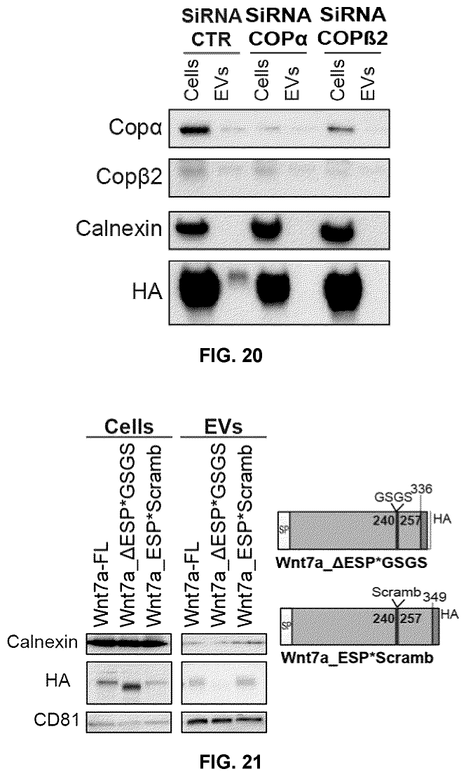

To directly assess the role of COPβ2 and COPα in mediating secretion of Wnt7a on EVs, Wnt7a-HA secreting HEK293T cells were transfected with siRNA to knock down COPβ2 or COPα (FIG. 20). Notably, knock down of COPβ2 and COPα resulted in a significant reduction in the amount of Wnt7a-HA detectable in isolated EVs (FIG. 20). Together, these results suggest a novel secretion mechanism via COPI vesicles wherein Wnt7a trafficking to EVs is regulated by interaction with components of the Coatomer complex.

The KR Motif within the ESP is Required for Binding to the Coatomer Complex

The presence of Coatomer proteins on EVs has been previously noted. Moreover, COPα and COPβ2 have been shown to bind with the positively charged motifs (KKxx, KxKxx, and in the case of β′-COP also RKxx) present in interacting proteins. Therefore, the role played by the positively charged motif present in the ESP was evaluated to mediate secretion of Wnt7a on EVs was evaluated.

First, a Wnt7a mutant was tested in which the ESP sequence was scrambled whilst maintaining the positively charged motifs (FIG. 49). Replacement of the ESP sequence (PVRASRNKRPTFLKIKKP) with the linker sequence GSGS (Wnt7a_ΔESP*GCGS) completely abrogated secretion on EVs. (FIG. 21) By contrast, replacing the ESP with a scrambled sequence (PNKKLASPRITFKPKRRV), which maintained the positively charged motifs (Wnt7a_ESP*Scramb), had no effect on Wnt7a-EVs secretion (FIG. 21).

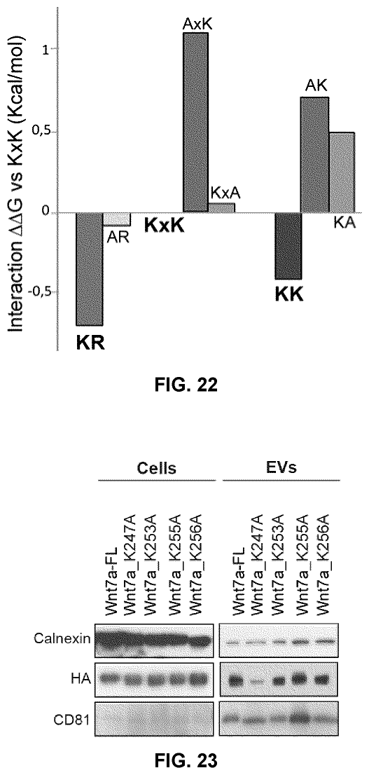

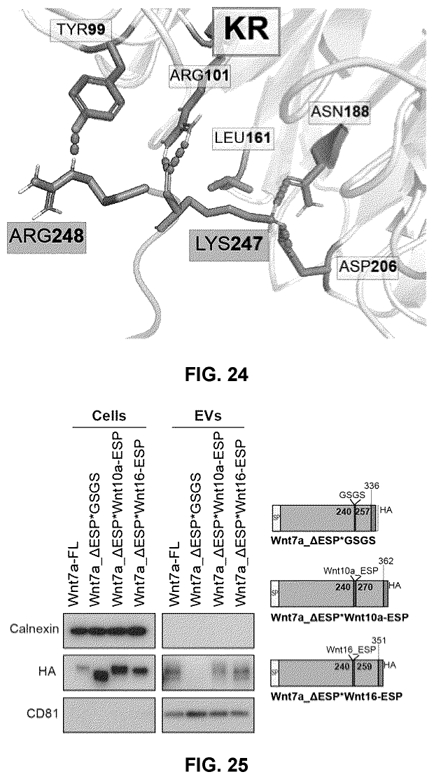

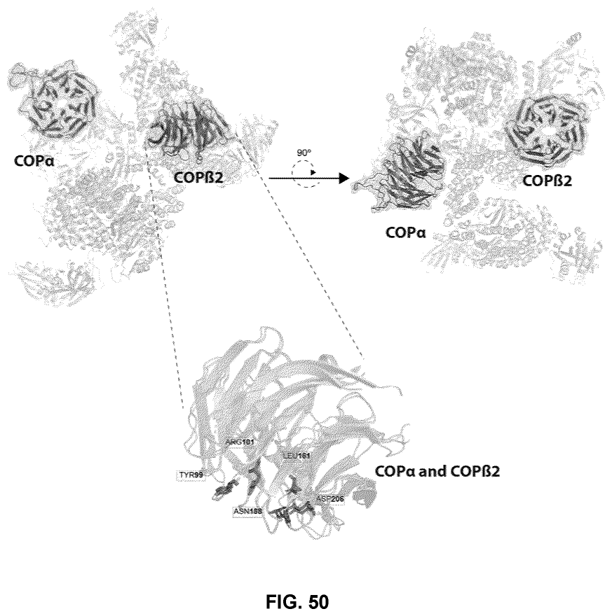

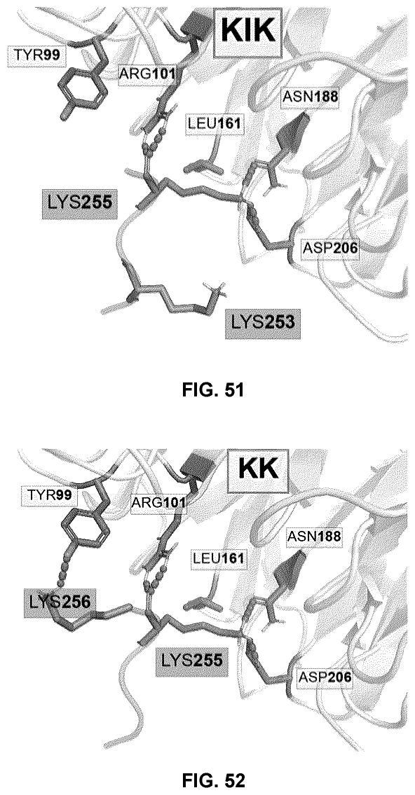

In silico 3D modeling of the Wnt7a interaction with COPβ2 suggests a stable interaction through hydrogen bonds and hydrophobic interactions with the positively charged Wnt7a motif with three different residues of COPβ2. Due to the presence of an identical binding pocket in COPα subunit (FIG. 50) the motif recognition analysis can be applied for both subunits. As a template, the crystallographically resolved interaction between COPβ2 and a peptide with the motif KxK (PDB id 2YNP) was used. The three positively charged motifs present on the ESP region of Wnt7a (KR, KIK, KK) were annealed onto the sequence of COPβ2 with FoldX BuildModel command. The interaction energy of the positively charged motifs with COPβ2 with respect to the crystallized KIK motif was then measured and results showed that both the KR (FIG. 24) and KK (FIG. 52) motifs are recognized, as is the original KxK motif (FIG. 51). This occurs through the binding of Arg248 of Wnt7a to the side chain of TYR99, while the interactions of the main chain CO group of Wnt7a-Lys247 with the side chain of ARG101 and of the side chain of Lys247 with the side chains of LEU161, ASN188 and ASP206 in the original motif are kept (FIG. 22). Conversely, interaction ablation, modeled by mutating the interacting residues to alanine, destabilized the interaction (FIG. 22). The fact that KR interaction appears stronger than KK is due to the different angle position of the hydrogen bond and the proximity of the interaction.

To empirically test the structural model, single point mutations were performed of the lysines residues to alanine across the ESP domain. Only the disruption of KR was found to impair the secretion of Wnt7a on EVs. Indeed, mutation of K256 that disrupts both positively charged motifs, KIK and KK, did not affect secretion of Wnt7a-EVs (FIG. 23). This data together confirms that COPβ2 and COPα regulate Wnt7a trafficking into EVs by interacting with the KR motif within the ESP.

### The Mechanism of EV Secretion is Conserved Across the Wnt Family

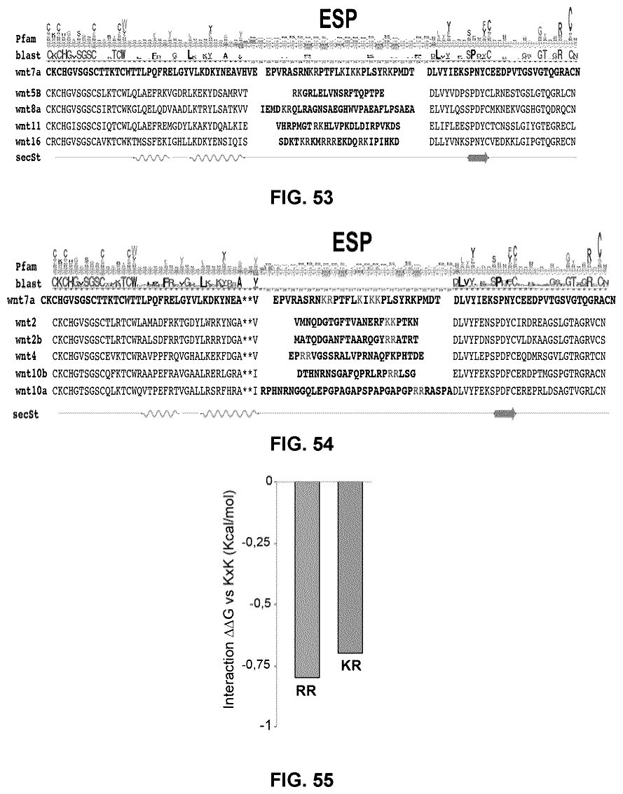

The ESP region corresponds with a linking peptide that connects the N- and C-terminal domains with a high variable length and sequence among the 19 human Wnt proteins (FIG. 53-54). Notably, the KR motif responsible for Coatomer interaction is also present in Wnt5b, Wnt8a, Wnt11 and Wnt16, suggesting the possibility of a conserved EVs secretion mechanism across the Wnt family (FIG. 53). Moreover, another positively charged motif, RR, is highly conserved in Wnt2b, Wnt4, Wnt10b, Wnt10a and Wnt16 proteins (FIG. 54). Indeed, in silico measurement of the RR motif interaction with COPβ2 suggested a slightly higher interaction affinity compared with KR motif (FIG. 55).

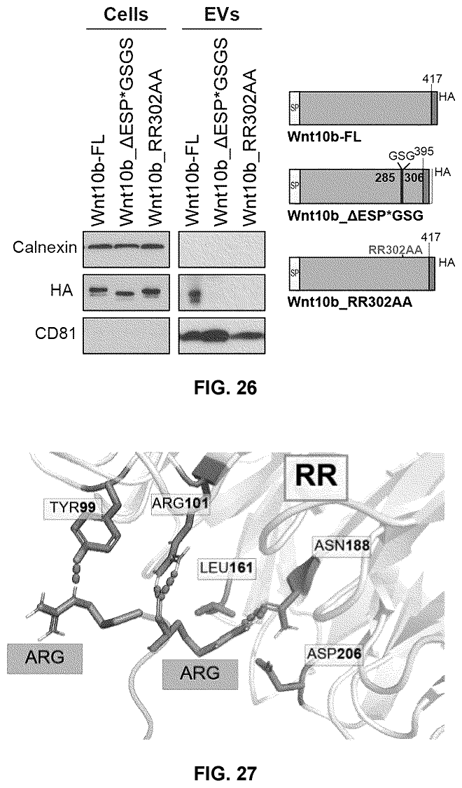

To test the ability of candidate ESPs from different Wnts to mediate secretion on EVs, the ESP of Wnt7a was replaced with either the ESP from Wnt10a, containing only the RR motif, or the ESP from Wnt16, that contains both motifs RR and KR. Both the Wnt10a and the Wnt16 ESP were compatible for efficient secretion on EVs (FIG. 25). Furthermore, deletion of the ESP from Wnt10b-EVs, or double mutation of its RR motif, completely abrogated secretion of Wnt10b on EVs (FIG. 26). Together these results strongly support the assertion that the direct binding of the Coatomer complex by Wnt family members via the KR motif present within the ESP domain represents a conserved mechanism that mediates the secretion and localization of Wnts on the surface of EVs.

### DISCUSSION

Here, the structural mechanism that targets Wnt proteins to the surface of EVs has been elucidated. A new role for COPI vesicles as mediators of Wnt secretion on EVs has been identified. It has been discovered that COPα and COPβ2 interact through their N-terminal β-propeller domains with a positively charged KR motif found in a loop within Wnt7a that has been termed ESP. This interaction mediates the targeting of Wnt7a to EVs, thus facilitating long-range signaling by Wnt7a. This dual requirement is interpreted as reflecting the changing interactions along the secretion pathway: first, the interaction with COPα in the cytosolic membrane of the Golgi Apparatus; and second, the interaction with COPβ2 in the cellular membrane. Interestingly, it was found that when COPβ2 is knocked down, COPα is also down regulated in EVs. This suggests that both proteins are required for the proper formation of the COPI vesicle and secretion of Wnt7a-EVs is abolished in the absence of either component.

Several groups have shown that Wnt secretion requires an interaction with Evi, a chaperone transmembrane protein that facilitates the secretion to the membrane. Moreover, it has been shown that Evi interacts with COPI vesicles to mediate the recycling of Evi and thus promote Wnt secretion. This data would suggest that Evi could also act as the linker be-tween Coatomer and Wnt facilitating the transfer to the membrane. However, recently it was confirmed that the interaction of Wnt to Evi it is through palmitoylation of Wnt. However, it was observed that palmitoylation was dispensable for Wnt7a secretion on EVs, ruling out the possibility of Evi mediating the Coatomer-dependent Wnt-EV secretion mechanism. Accordingly, no interaction of Wnt7a with Evi was detected by Bio-ID. The experiments suggest an alternative Coatomer-dependent mechanism for Wnt secretion on EVs, where COPI vesicles mediate intracellular trafficking of Wnt7a from the Golgi apparatus surface to the cellular membrane. Indeed, the results indicate that upon mutation of the ESP, secretion as a free protein is enhanced to the detriment of Wnt-EV secretion, thus confirming that both modes of secretion function independently. Exosomal secretion mechanism would compensate for the inability of free Wnt to signal long-range and provide for fast distal-range diffusion after acute muscle injury. This data reinforces the concept of independent co-existing secretion pathways and the ability of the cell to switch from one to another based on cellular homeostasis.

The N-terminal SP has long been understood to be required for extracellular protein secretion. Also, it has been assumed that proteins targeted for exosomal secretion are endocytosed directly from the cell membrane before being subsequently transferred back to the Multivesicular Body (MVB). Importantly, it has been found that the SP is not required for secretion of Wnt7a on EVs. Therefore, this data suggests that Wnt7a trafficking onto EVs occurs inside of the cell and not after being secreted as a free protein, which is later endocytosed. Since Wnt proteins have been described to be secreted through the classical ER-Golgi pathway it is unlikely that Wnt located in the luminal side of the Golgi could interact with the cytosolic Coatomer proteins. Therefore, a novel mechanism is suggested, wherein proteins would bypass the classical pathway ER-Golgi pathway (FIG. 7).

The BioID data has shown an enrichment on Sec63, a chaperone that facilitates targeting of proteins bearing a SP into the Sec61 channel at the ER. This finding is consistent with the notion that Wnt proteins are translated in the cytosol and translocated to the ER with the assistance of Sec63. The fraction of Wnt remaining in the cytosol, however, would be available for direct cytosolic interaction with COPα at the Golgi. This new role for COPI vesicles is reinforced by the lack of any retrograde signal within the EBP sequence, that could relate this interaction with a retrograde mechanism. Also, the results showed that neither palmitoylation nor the SP is required for Wnt-EV secretion. Indeed, other Wnt proteins, such as WntD, have been previously shown to be secreted without palmitoylation. Furthermore, it has been shown that Wnt7a is fully bioactive upon secretion on EVs, as several authors have previously shown for other Wnts. Therefore maturation through the ER-Golgi classical pathway seems to be dispensable for specific Wnt-EV secretion and bioactivity.

Studies have previously identified the involvement of COPI vesicles in endosome trafficking. In Drosophila, knockdown of COPα or COPβ2 results in adult flies that display notched wings, suggesting an essential role for COPI vesicles in Wg secretion. Protein secretion pathways have been described that function independently of the classical ER-GA pathway. Therefore, the data is consistent with the assertion that Wnt7a secretion on EVs is occurring via a Coatomer-dependent leaderless secretion pathway rather than the classical ER-GA pathway. However, the mechanism that specifically regulates COPI vesicles cargo into MVB for protein-EV secretion needs further investigation.

The same linker region that forms the ESP has been implicated in other Wnt7a functions. The Reck receptor binds Wnt7a through the same region that encodes the ESP to form a signalosome that induces canonical Wnt7a-Fzd signaling. Moreover, Wnt7a similarly binds the canonical Frizzled co-receptor LRP6 through the ESP sequence. Together these findings suggest that this unstructured loop acts as an intrinsically disordered protein, to coordinate different functions possibly regulated by combinatorial posttranslational modifications. Notably, Reck was not detected in the BioID assays, and Reck expression by immunoblot analysis were not detected. Together, these data reinforce the notion that multiple mechanisms act on Wnt-signaling in different cell types to enforce distinct signaling outcomes.

It has been found that equivalent ESP sequences are conserved in several Wnts to mediate secretion on EVs. Further, it has been found that the mechanism of action is conserved through interaction with non-canonical charged amino acid motifs such us RR. It has been shown that Wnt secretion on EVs can be abrogated by mutating a single amino acid within the ESP without disrupting other types of secretion.

A novel role for COPI vesicles has been defined, which involves Wnt-Coatomer protein binding to target Wnt proteins for EVs secretion. The sequence requirements of ESPs and their coatomer binding motifs (CBMs) have been defined and it has been shown that a similar mechanism is involved in EVs secretion of multiple Wnts. These experiments suggest that systemic delivery of Wnt7a loaded on exosomes represents a potential therapy for neuromuscular diseases such as DMD. Moreover, the use of ESPs and/or CMBs to direct the display of other cargo proteins on the surface of exosomes opens the door for multiple therapeutic applications involving targeting of recombinant cargo proteins to EVs. In particular the unexpected involvement of COPI in Wnt trafficking to EVs suggest that other known CBMs, such as KR, KK, KxK (which bind to α-COP and/or β′-COP) will also be useful in this regard, as well as the motif FFxxBB (which binds to γ-COP). The significance of RR as a CBM, as described herein, also appears to be new. It is expected that these discoveries will serve as a basis for recombinant delivery systems.

### Example 2

### ESP/CBM Mutation Increases Free Wnt7a Protein

As illustrated in FIG. 10, Wnt7a proteins with an ESP replaced by a linker region have disrupted binding to EVs, yielding a displacement of Wnt7a to free protein secretion. The immunoblots shown in FIG. 10 show that the totally of Wnt7a in the full cell lysate (cells) is displaced from the EV isolated EV fraction (EVs) to the Free Protein (FP) fraction, showing that ESP-containing Wnt7a (Wnt7a-FL) is directed to EVs, whereas conversely, the disrupted ESP (Wnt7a-ΔESP*GSGS) yields an increase in free Wnt7a protein. Table 6 below shows a quantification of FIG. 10, demonstrating the displacement of Wnt7a from EVs to free protein when the ESP is replaced with a linker region with a p value of 0.00044, with the experiment carried out in triplicate.

TABLE 6
ESP distribution between EVs and Free Protein fractions

| | EV Fraction | | Free Protein Fraction | |
|---|---|---|---|---|
| | Wnt7a- | Wnt7a- | Wnt7a- | Wnt7a- |
| | FL | ΔESP*GSGS | FL | ΔESP*GSGS |
| Selection pattern | 83.4 | 7.2 | 6.13 | 86.7 |
| Distribution (%) | | | | |

Extracellular Vesicles Signal Peptide (ESP) Deletion (Replacement with Linker) Increases Extracellular Secretion of Free Wnt7a Protein

As illustrated in FIG. 31, Wnt7a proteins with an ESP replaced by a linker region have disrupted binding to EVs, yielding a displacement of Wnt7a to free protein secretion from the cell into the cell media (FP permeate). Components of cells expressing Wnt7a-FP and Wnt7a with ESP replaced with a linker region were analyzed via tangential flow filtration techniques enabling the separate of full cells, EVs from the cell media containing secreted proteins. Replacement of the ESP redirects Wnt7a protein from EVs to increase cell secretion of Wnt7a. The immunoblots shown in FIG. 31 show that Wnt7a protein is displaced from the EV isolated EV fraction (EVs) to the Cell Media (FP permeate), showing that ESP-containing Wnt7a (Wnt7a-FL) is directed to EVs, whereas conversely, the disrupted ESP yields an increase in free Wnt7a protein secreted from the cells. Table 7 below shows a quantification of FIG. 10, demonstrating the displacement of Wnt7a from EVs to free protein when the ESP is replaced with a linker region with a p value of 0.11 for the experiment carried out in triplicate.

TABLE 7
ESP distribution between EVs and Free Protein Secretion

| | EV Fraction | |
|---|---|---|
| | | Wnt7a- |
| | Wnt7a-FL | ΔESP*GSGS |
| Wnt7a | 0.13 | 0.04 |
| expression per | | |
| ug of total protein | | |

The disruption in the above cases was deletion of the ESP and replacement with a GSGS linker. However, it is clear that there would be other ways to reduce or disrupt ESP activity to achieve a similar increase in free protein, such as by mutation of one or more key residues in the CBM(s) of a given Wnt, or by making other deletions in the ESP. These approaches should be useful to generate free Wnts, including for therapeutic applications.

### REFERENCES

1. J. C. Gross, V. Chaudhary, K. Bartscherer, M. Boutros, Active Wnt proteins are secreted on exosomes. Nat. Cell Biol. 14, 1036-1045 (2012).
2. V. Luga, L. Zhang, A. M. Viloria-Petit, A. A. Ogunjimi, M. R. Inanlou, E. Chiu, M. Buchanan, A. N. Hosein, M. Basik, J. L. Wrana, Exosomes mediate stromal mobilization of autocrine Wnt-PCP signaling in breast cancer cell migration. Cell. 151, 1542-1556 (2012).
3. K. Menck, F. Klemm, J. C. Gross, T. Pukrop, D. Wenzel, C. Binder, Induction and transport of Wnt 5a during macrophage-induced malignant invasion is mediated by two types of extracellular vesicles. Oncotarget. 4, 2057-66 (2013).
4. K. Beckett, S. Monier, L. Palmer, C. Alexandre, H. Green, G. Raposo, P. Thibault, R. Le Borgne, J. Vincent, Europe PMC Funders Group Drosophila Wingless is loaded on exosome-like vesicles but forms a gradient in an exosome-independent manner. 14, 82-96 (2015).
5. A. M. Pani, B. Goldstein, Direct visualization of a native wnt in vivo reveals that a long-range Wnt gradient forms by extracellular dispersal. Elife. 7, 1-22 (2018).
6. M. Eubelen, N. Bostaille, P. Cabochette, A. Gauquier, P. Tebabi, A. C. Dumitru, M. Koehler, P. Gut, D. Alsteens, D. Y. R. Stainier, A. Garcia-Pino, B. Vanhollebeke, A molecular mechanism for Wnt ligand-specific signaling. Science (80-.). 361, 1-22 (2018).
7. M. L. H. Chu, V. E. Ahn, H. J. Choi, D. L. Daniels, R. Nusse, W. I. Weis, Structural studies of wnts and identification of an LRP6 binding site. Structure. 21, 1235-1242 (2013).
8. A. Shevchenko, H. Tomas, J. Havliš, J. V. Olsen, M. Mann, In-gel digestion for mass spectrometric characterization of proteins and proteomes. Nat. Protoc. 1, 2856-2860 (2007).
9. G. V. Los, L. P. Encell, M. G. McDougall, D. D. Hartzell, N. Karassina, C. Zimprich, M. G. Wood, R. Learish, R. F. Ohana, M. Urh, D. Simpson, J. Mendez, K. Zimmerman, P. Otto, G. Vidugiris, J. Zhu, A. Darzins, D. H. Klaubert, R. F. Bulleit, K. V. Wood, HaloTag: A novel protein labeling technology for cell imaging and protein analysis. ACS Chem. Biol. 3, 373-382 (2008). 11. R. Beck, M. Ravet, F. T. Wieland, D. Cassel, The COPI system: Molecular mechanisms and function. FEBS Lett. 583, 2701-2709 (2009).
10. J. Bethune, F. T. Wieland, Assembly of COPI and COPII Vesicular Coat Proteins on Membranes. Annu. Rev. Biophys. 47, 63-83 (2018).
11. S. Chun, S. Ahn, C. H. Yeom, S. Park, Exosome proteome of U-87MG glioblastoma cells. Biology (Basel). 5, 1-11 (2016).
12. B. J. Tauro, D. W. Greening, R. A. Mathias, S. Mathivanan, H. Ji, R. J. Simpson, Two distinct populations of exosomes are released from LIM1863 colon carcinoma cell-derived organoids. Mol. Cell. Proteomics. 12, 587-598 (2013).
13. W. Ma, J. Goldberg, Rules for the recognition of dilysine retrieval motifs by coatomer. EMBO J. 32, 926-937 (2013).
14. L. P. Jackson, M. Lewis, H. M. Kent, M. A. Edeling, P. R. Evans, R. Duden, D. J. Owen, Molecular Basis for Recognition of Dilysine Trafficking Motifs by COPI. Dev. Cell. 23, 1255-1262 (2 012).
15. R. Nygaard, J. Yu, J. Kim, D. R. Ross, G. Parisi, O. B. Clarke, D. M. Virshup, F. Mancia, Structural Basis of WLS/Evi-Mediated Wnt Transport and Secretion. Cell. 184, 194-206.e14 (2021).
16. D. Akopian, K. Shen, X. Zhang, S. Shan, Signal Recognition Particle: An Essential Protein-Targeting Machine. Annu. Rev. Biochem. 82, 693-721 (2013).
17. M. Razi, E. Y. W. Chan, S. A. Tooze, Early endosomes and endosomal coatomer are required for Autophagy. J. Cell Biol. 185, 305-321 (2009).
18. G. Gabriely, R. Kama, J. E. Gerst, Involvement of Specific COPI Subunits in Protein Sorting from the Late Endosome to the Vacuole in Yeast. Mol. Cell. Biol. 27, 526-540 (2007).
19. P. Zhang, L. Zhou, C. Pei, X. Lin, Z. Yuan, Dysfunction of Wntless triggers the retrograde Golgi-to-ER transport of Wingless and induces ER stress. Sci. Rep. 6, 1-11 (2016).
20. V. Malhotra, Unconventional protein secretion: An evolving mechanism. EMBO J. 32, 1660-1664 (2013).
21. L. Alvarez-Erviti, Y. Seow, H. Yin, C. Betts, S. Lakhal, M. J. A Wood, Delivery of siRNA to the mouse brain by systemic injection of targeted exosomes. Nat. Biotechnol. 29, 3-4 (2011).
22. O. Moreno-Gonzalo, I. Fernandez-Delgado, F. Sanchez-Madrid, Post-translational add-ons mark the path in exosomal protein sorting. Cell. Mol. Life Sci. 75, 1-19 (2018).
23. M. Vallon, K. Yuki, T. D. Nguyen, J. Chang, J. Yuan, D. Siepe, Y. Miao, M. Essler, M. Noda, K. C. Garcia, C. J. Kuo, A RECK-WNT7 Receptor-Ligand Interaction Enables Isoform-Specific Regulation of Wnt Bioavailability. Cell Rep. 25, 339-349.e9 (2018).
24. H. Hirai, K. Matoba, E. Mihara, T. Arimori, J. Takagi, Crystal structure of a mammalian Wnt-frizzled complex. Nat. Struct. Mol. Biol. 26, 372-379 (2019).

All references referred to herein are incorporated by reference in their entireties.

In the preceding description, for purposes of explanation, numerous details are set forth in order to provide a thorough understanding of the embodiments. However, it will be apparent to one skilled in the art that these specific details are not required. In other instances, well-known electrical structures and circuits are shown in block diagram form in order not to obscure the understanding. For example, specific details are not provided as to whether the embodiments described herein are implemented as a software routine, hardware circuit, firmware, or a combination thereof.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

1. An extracellular vesicle (EV) comprising:
coat protein complex 1 (COPI), and
a recombinant EV-directed polypeptide comprising:
a cargo polypeptide, and
an extracellular vesicle signal peptide (ESP) comprising a coatomer binding motif (CBM),
wherein the cargo polypeptide is tethered to an external surface of the EV via the coatomer binding motif.

2. The EV of claim 1, wherein the ESP is for binding to a α-COP, β′-COP, or γ-COP subunit of the COPI.

3. (canceled)

4. The EV of claim 2, wherein the coatomer binding motif comprises a two- or three-amino acid motif comprising two positively charged amino acids residues.

5. The EV of claim 4, wherein the two- or three-amino acid motif comprises KR, KK, KxK, RK, or RR, wherein x is any amino acid.

6. (canceled)

7. The EV of claim 1, wherein the CBM is located in the EV-directed polypeptide:
in an unstructured loop of the cargo polypeptide,
in an unstructured tail that is positioned C-terminally with respect to the cargo polypeptide, or
in an unstructured leader sequence that is positioned at N-terminally with respect to the cargo polypeptide, wherein the EV-directed polypeptide lacks a signal peptide.

8. The EV of claim 1, wherein the ESP is at least 16 amino acids in length, more preferably from 18 to 34 amino acids in length.

9-13. (canceled)

14. The EV of claim 1, wherein the cargo protein is a therapeutic polypeptide, and wherein the therapeutic protein polypeptide an antibody or an antigen-binding fragment thereof, an enzyme, a cytotoxic polypeptide, an antigen, or a protein that is deficient in disease state.

15. A recombinant extracellular vesicle (EV)-directed polypeptide comprising:
a cargo polypeptide, and
an extracellular vesicle signal peptide (ESP) comprising a coatomer binding motif (CBM).

16. The recombinant EV-directed polypeptide of claim 15, wherein the ESP is for binding to a α-COP, β′-COP, or γ-COP of coat protein complex 1 (COPI).

17. (canceled)

18. The recombinant EV-directed polypeptide of claim 15, wherein the coatomer binding motif comprises a two- or three-amino acid motif comprising two positively charged amino acids residues.

19. The recombinant EV-directed polypeptide of claim 18, wherein the two- or three-amino acid motif comprises KR, KK, KxK, RK, or RR, wherein x is any amino acid.

20. (canceled)

21. The recombinant EV-directed polypeptide of claim 15, wherein the CBM is located in the EV-directed polypeptide:
in an unstructured loop of the cargo polypeptide,
in an unstructured tail that is positioned C-terminally with respect to the cargo polypeptide, or
in an unstructured leader sequence that is positioned at N-terminally with respect to the cargo polypeptide, wherein the EV-directed polypeptide lacks a signal peptide.

22. The recombinant EV-directed polypeptide of claim 15, wherein the ESP is at least 16 amino acids in length, more preferably from 18 to 34 amino acids in length.

23-27. (canceled)

28. The recombinant EV-directed polypeptide of claim 15, wherein the cargo protein is a therapeutic polypeptide, and wherein the therapeutic polypeptide comprises an antibody or an antigen-binding fragment thereof, an enzyme, an antigen, a cytotoxic protein, or a protein that is deficient in disease state.

29. A nucleic acid molecule encoding the recombinant EV-directed polypeptide as defined in claim 15.

30. A viral particle comprising the nucleic acid as defined in claim 29.

31. A recombinant host cell comprising the nucleic acid as defined in claim 29.

32. (canceled)

33. A use of the EV as defined in claim 1 for delivery of the cargo polypeptide to a cell.

34. (canceled)

35. (canceled)

36. A method of delivering a cargo polypeptide to a cell comprising contacting the cell with the EV as defined in claim 1.

37. The recombinant extracellular vesicle (EV)-directed polypeptide of claim 15, wherein:
the cargo polypeptide comprises a skeletal muscle targeting moiety comprising a Wnt family polypeptide, or polypeptide at least 90% identical thereto.

38.-72. (canceled)


SEQUENCE LISTING (122 sequences)

SEQ ID NO: 1 - protein, 360 aa, Artificial Sequence
  1-Wnt2
MNAPLGGIWLWLPLLLTWLTPEVNSSWWYMRATGGSSRVMCDNVPGLVSSQRQLCHRHPDVMRAISQGVAEWTAECQHQFRQHRWNCNTLDRDHSLFGRVLLRSSRESAFVYAISSAGVVFAITRACSQGEVKSCSCDPKKMGSAKDSKGIFDWGGCSDNIDYGIKFARAFVDAKERKGKDARALMNLHNNRAGRKAVKRFLKQECKCHGVSGSCTLRTCWLAMADFRKTGDYLWRKYNGAIQVVMNQDGTGFTVANERFKKPTKNDLVYFENSPDYCIRDREAGSLGTAGRVCNLTSRGMDSCEVMCCGRGYDTSHVTRMTKCGCKFHWCCAVRCQDCLEALDVHTCKAPKNADWTTAT

SEQ ID NO: 2 - protein, 391 aa, Artificial Sequence
  2-Wnt2b
MLRPGGAEEAAQLPLRRASAPVPVPSPAAPDGSRASARLGLACLLLLLLLTLPARVDTSWWYIGALGARVICDNIPGLVSRQRQLCQRYPDIMRSVGEGAREWIRECQHQFRHHRWNCTTLDRDHTVFGRVMLRSSREAAFVYAISSAGVVHAITRACSQGELSVCSCDPYTRGRHHDQRGDFDWGGCSDNIHYGVRFAKAFVDAKEKRLKDARALMNLHNNRCGRTAVRRFLKLECKCHGVSGSCTLRTCWRALSDFRRTGDYLRRRYDGAVQVMATQDGANFTAARQGYRRATRTDLVYFDNSPDYCVLDKAAGSLGTAGRVCSKTSKGTDGCEIMCCGRGYDTTRVTRVTQCECKFHWCCAVRCKECRNTVDVHTCKAPKKAEWLDQT

SEQ ID NO: 3 - protein, 351 aa, Artificial Sequence
  3-Wnt4
MSPRSCLRSLRLLVFAVFSAAASNWLYLAKLSSVGSISEEETCEKLKGLIQRQVQMCKRNLEVMDSVRRGAQLAIEECQYQFRNRRWNCSTLDSLPVFGKVVTQGTREAAFVYAISSAGVAFAVTRACSSGELEKCGCDRTVHGVSPQGFQWSGCSDNIAYGVAFSQSFVDVRERSKGASSSRALMNLHNNEAGRKAILTHMRVECKCHGVSGSCEVKTCWRAVPPFRQVGHALKEKFDGATEVEPRRVGSSRALVPRNAQFKPHTDEDLVYLEPSPDFCEQDMRSGVLGTRGRTCNKTSKAIDGCELLCCGRGFHTAQVELAERCSCKFHWCCFVKCRQCQRLVELHTCR

SEQ ID NO: 4 - protein, 359 aa, Artificial Sequence
  4-Wnt5b
MPSLLLLFTAALLSSWAQLLTDANSWWSLALNPVQRPEMFIIGAQPVCSQLPGLSPGQRKLCQLYQEHMAYIGEGAKTGIKECQHQFRQRRWNCSTADNASVFGRVMQIGSRETAFTHAVSAAGVVNAISRACREGELSTCGCSRTARPKDLPRDWLWGGCGDNVEYGYRFAKEFVDAREREKNFAKGSEEQGRVLMNLQNNEAGRRAVYKMADVACKCHGVSGSCSLKTCWLQLAEFRKVGDRLKEKYDSAAAMRVTRKGRLELVNSRFTQPTPEDLVYVDPSPDYCLRNESTGSLGTQGRLCNKTSEGMDGCELMCCGRGYNQFKSVQVERCHCKFHWCCFVRCKKCTEIVDQYICK

SEQ ID NO: 5 - protein, 349 aa, Artificial Sequence
  5-Wnt7a
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 6 - protein, 351 aa, Artificial Sequence
  6-Wnt8a
MGNLFMLWAALGICCAAFSASAWSVNNFLITGPKAYLTYTTSVALGAQSGIEECKFQFAWERWNCPENALQLSTHNRLRSATRETSFIHAISSAGVMYIITKNCSMGDFENCGCDGSNNGKTGGHGWIWGGCSDNVEFGERISKLFVDSLEKGKDARALMNLHNNRAGRLAVRATMKRTCKCHGISGSCSIQTCWLQLAEFREMGDYLKAKYDQALKIEMDKRQLRAGNSAEGHWVPAEAFLPSAEAELIFLEESPDYCTCNSSLGIYGTEGRECLQNSHNTSRWERRSCGRLCTECGLQVEERKTEVISSCNCKFQWCCTVKCDQCRHVVSKYYCARSPGSAQSLGKGSA

SEQ ID NO: 7 - protein, 417 aa, Artificial Sequence
  7-Wnt10a
MGSAHPRPWLRLRPQPQPRPALWVLLFFLLLLAAAMPRSAPNDILDLRLPPEPVLNANTVCLTLPGLSRRQMEVCVRHPDVAASAIQGIQIAIHECQHQFRDQRWNCSSLETRNKIPYESPIFSRGFRESAFAYAIAAAGVVHAVSNACALGKLKACGCDASRRGDEEAFRRKLHRLQLDALQRGKGLSHGVPEHPALPTASPGLQDSWEWGGCSPDMGFGERFSKDFLDSREPHRDIHARMRLHNNRVGRQAVMENMRRKCKCHGTSGSCQLKTCWQVTPEFRTVGALLRSRFHRATLIRPHNRNGGQLEPGPAGAPSPAPGAPGPRRRASPADLVYFEKSPDFCEREPRLDSAGTVGRLCNKSSAGSDGCGSMCCGRGHNILRQTRSERCHCRFHWCCFVVCEECRITEWVSVCK

SEQ ID NO: 8 - protein, 389 aa, Artificial Sequence
  8-Wnt10b
MLEEPRPRPPPSGLAGLLFLALCSRALSNEILGLKLPGEPPLTANTVCLTLSGLSKRQLGLCLRNPDVTASALQGLHIAVHECQHQLRDQRWNCSALEGGGRLPHHSAILKRGFRESAFSFSMLAAGVMHAVATACSLGKLVSCGCGWKGSGEQDRLRAKLLQLQALSRGKSFPHSLPSPGPGSSPSPGPQDTWEWGGCNHDMDFGEKFSRDFLDSREAPRDIQARMRIHNNRVGRQVVTENLKRKCKCHGTSGSCQFKTCWRAAPEFRAVGAALRERLGRAIFIDTHNRNSGAFQPRLRPRRLSGELVYFEKSPDFCERDPTMGSPGTRGRACNKTSRLLDGCGSLCCGRGHNVLRQTRVERCHCRFHWCCYVLCDECKVTEWVNVCK

SEQ ID NO: 9 - protein, 354 aa, Artificial Sequence
  9-Wnt11
MRARPQVCEALLFALALQTGVCYGIKWLALSKTPSALALNQTQHCKQLEGLVSAQVQLCRSNLELMHTVVHAAREVMKACRRAFADMRWNCSSIELAPNYLLDLERGTRESAFVYALSAAAISHAIARACTSGDLPGCSCGPVPGEPPGPGNRWGGCADNLSYGLLMGAKFSDAPMKVKKTGSQANKLMRLHNSEVGRQALRASLEMKCKCHGVSGSCSIRTCWKGLQELQDVAADLKTRYLSATKVVHRPMGTRKHLVPKDLDIRPVKDSELVYLQSSPDFCMKNEKVGSHGTQDRQCNKTSNGSDSCDLMCCGRGYNPYTDRVVERCHCKYHWCCYVTCRRCERTVERYVCK

SEQ ID NO: 10 - protein, 365 aa, Artificial Sequence
  10-Wnt16
MDRAALLGLARLCALWAALLVLFPYGAQGNWMWLGIASFGVPEKLGCANLPLNSRQKELCKRKPYLLPSIREGARLGIQECGSQFRHERWNCMITAAATTAPMGASPLFGYELSSGTKETAFIYAVMAAGLVHSVTRSCSAGNMTECSCDTTLQNGGSASEGWHWGGCSDDVQYGMWFSRKFLDFPIGNTTGKENKVLLAMNLHNNEAGRQAVAKLMSVDCRCHGVSGSCAVKTCWKTMSSFEKIGHLLKDKYENSIQISDKTKRKMRRREKDQRKIPIHKDDLLYVNKSPNYCVEDKKLGIPGTQGRECNRTSEGADGCNLLCCGRGYNTHVVRHVERCECKFIWCCYVRCRRCESMTDVHTCK

SEQ ID NO: 11 - protein, 349 aa, Mus musculus
MTRKARRCLGHLFLSLGIVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 12 - protein, 349 aa, Rattus norvegicus
MTRKARRCLGHLFLSLGIVYLRIGDFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 13 - protein, 349 aa, Canis lupus familiaris
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEVYTCK

SEQ ID NO: 14 - protein, 349 aa, Bos taurus
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPAFLKIKKPLSYRKPMDTELVYIEKSPSYCEEDPATGSVGTQGRACNKTAPQASGCDLMCCGRGYNTHQYARVWQCNCKFHWCCYVKCNTCSERTEVYTCK

SEQ ID NO: 15 - protein, 349 aa, Macaca mulatta
MNRKARRCLGHLFLSLGMVYLRIGGFSTVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 16 - protein, 349 aa, Gallus gallus
MNRKTRRWIFHIFLSLGIVYIKIGGFSSVVALGASIICNKIPGLAPRQRAICQSRPDAIIVIGEGSQMGINECQFQFRNGRWNCSALGERTVFGKELKVGSREAAFTYAIIAAGVAHAITAACTQGNLSDCGCDKEKQGQYHKEEGWKWGGCSADIRYGIGFAKVFVDAREIKQNARTLMNLHNNEAGRKILEENMKLECKCHGVSGSCTTKTCWTTLPKFRELGYILKDKYNEAVQVEPVRASRNKRPTFLKIKKPLSYRKPMDTDLVYIEKSPNYCEEDPVTGSVGTQGRMCNKTAQQSNGCDLMCCGRGYNTHQYSRVWQCNCKFHWCCYVKCNTCSERTEVYTCK

SEQ ID NO: 17 - protein, 248 aa, Xenopus tropicalis
GSREAAFMYAIIAAGVAHAITTACTQGNMSDCGCDKEKQGQFHREEGWKWGGCSADIRYGIGFSKVFVDAREIKQNARTLMNLHNNEAGRRILKESMKSECKCHGVSGSCTTKTCWTTLPKFRELGAILRDKYNEAIQVEPVRASRNKRPTFLKIKNSYRKPMDTDLVYIEKSPNYCEEDPMTGSVGTQGRLCNKTAQHTSSCDLMCCGRGYNTHQYSRVWQCNCKFHWCCYVKCNTCSERTEVFTCK

SEQ ID NO: 18 - protein, 349 aa, Danio rerio
MSRKTRRWIFHIFLCLGIIYLKIGGFSSVVALGASIICNKIPGLAPRQRTICQSRPDAIIVIGEGAQMGINECQFQFKNGRWNCSALGERTVFGKELKVGSKEAAFTYAIIAAGVAHAITAACTQGTLSGCGCDKEKQGFYNQEEGWKWGGCSADIRYGLSFSKVFLDAREIKQNARTLMNLHNNEVGRKILEKNMRLECKCHGVSGSCTTKTCWTTLPKFRQLGYILKERYNHAVHVEPVRASRNKRPAFLKVKKPYSYRKPMDTDLVYIEKSPNYCEADPVTGSMGTQGRICNKTAQHTNGCDLMCCGRGYNTHQYSRVWQCNCKFLWCCYVKCNTCSERTEVYTCK

SEQ ID NO: 19 - protein, 177 aa, Artificial Sequence
  19-Wnt7a_Delta32-212
MNRKARRCLGHLFLSLGMVYLRIGGFSSVVATCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCKYPYDVPDYA

SEQ ID NO: 20 - DNA, 42 bp, Artificial Sequence
  Wnt7a_Delta-32-212 F-Primer
gcttctcctcagtggtagctacgtgctggaccacactgccac

SEQ ID NO: 21 - DNA, 58 bp, Artificial Sequence
  21-Wnt7a_Delta-32-212 R-Primer
aagaattctcaagcgtaatctggaacatcgtatgggtacttgcacgtgtacatctccg

SEQ ID NO: 22 - protein, 240 aa, Artificial Sequence
  22-Wnt7a_Delta32-149
MNRKARRCLGHLFLSLGMVYLRIGGFSSVVAGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCKYPYDVPDYA

SEQ ID NO: 23 - DNA, 125 bp, Artificial Sequence
  23-Wnt7a_Delta32-149 F-Primer
aaggatccaccatgaaccggaaagcgcggcgctgcctgggccacctctttctcagcctgggcatggtctacctccggatcggtggcttctcctcagtggtagctggtggctgctctgccgacatc

SEQ ID NO: 24 - DNA, 58 bp, Artificial Sequence; identical to SEQ ID NO: 21
  24-Wnt7a_Delta32-149 R-Primer

SEQ ID NO: 25 - protein, 290 aa, Artificial Sequence
  25-Wnt7a_Delta32-99
MNRKARRCLGHLFLSLGMVYLRIGGFSSVVAGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCKYPYDVPDYA

SEQ ID NO: 26 - DNA, 125 bp, Artificial Sequence
  26-Wnt7a_Delta32-99 F-Primer
aaggatccaccatgaaccggaaagcgcggcgctgcctgggccacctctttctcagcctgggcatggtctacctccggatcggtggcttctcctcagtggtagctgggagccgggaggctgcgttc

SEQ ID NO: 27 - DNA, 58 bp, Artificial Sequence; identical to SEQ ID NO: 21
  27-Wnt7a_Delta32-99 R-Primer

SEQ ID NO: 28 - protein, 340 aa, Artificial Sequence
  28-Wnt7a_Delta32-49
MNRKARRCLGHLFLSLGMVYLRIGGFSSVVAAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCKYPYDVPDYA

SEQ ID NO: 29 - DNA, 128 bp, Artificial Sequence
  29-Wnt7a_Delta32-49 F-Primer
aaggatccaccatgaaccggaaagcgcggcgctgcctgggccacctctttctcagcctgggcatggtctacctccggatcggtggcttctcctcagtggtagctgcgatctgccagagccggcccgac

SEQ ID NO: 30 - DNA, 58 bp, Artificial Sequence; identical to SEQ ID NO: 21
  30-Wnt7a_Delta32-49 R-Primer

SEQ ID NO: 31 - protein, 221 aa, Artificial Sequence
  31-Wnt7a_Delta213-349
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKYPYDVPDYA

SEQ ID NO: 32 - DNA, 34 bp, Artificial Sequence
  32-Wnt7a_Delta213-349 F-Primer
aaggatccaccatgaaccggaaagcgcggcgctg

SEQ ID NO: 33 - DNA, 60 bp, Artificial Sequence
  33-Wnt7a_Delta213-349 R-Primer
aagaattctcaagcgtaatctggaacatcgtatgggtacttggtggtgcacgagcctgac

SEQ ID NO: 34 - protein, 258 aa, Artificial Sequence
  34-Wnt7a_Delta251-349
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPYPYDVPDYA

SEQ ID NO: 35 - DNA, 34 bp, Artificial Sequence; identical to SEQ ID NO: 32
  35-Wnt7a_Delta251-349 F-Primer

SEQ ID NO: 36 - DNA, 60 bp, Artificial Sequence
  36-Wnt7a_Delta251-349 R-Primer
aagaattctcaagcgtaatctggaacatcgtatgggtaggtgggccgcttgttgcggctg

SEQ ID NO: 37 - protein, 309 aa, Artificial Sequence
  37-Wnt7a_Delta301-349
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQYPYDVPDYA

SEQ ID NO: 38 - DNA, 34 bp, Artificial Sequence; identical to SEQ ID NO: 32
  38-Wnt7a_Delta301-349 F-Primer

SEQ ID NO: 39 - DNA, 60 bp, Artificial Sequence
  39-Wnt7a_Delta301-349 R-Primer
aagaattctcaagcgtaatctggaacatcgtatgggtactggggagccgtcttgttgcag

SEQ ID NO: 40 - protein, 241 aa, Artificial Sequence
  40-Wnt7a_Delta32-99_Delta301-349
MNRKARRCLGHLFLSLGMVYLRIGGFSSVVAGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQYPYDVPDYA

SEQ ID NO: 41 - DNA, 125 bp, Artificial Sequence; identical to SEQ ID NO: 26
  41-Wnt7a_Delta32-99_Delta301-349 F-Prime

SEQ ID NO: 42 - DNA, 60 bp, Artificial Sequence; identical to SEQ ID NO: 39
  42-Wnt7a_Delta32-99_Delta301-349 R-Primer

SEQ ID NO: 43 - protein, 210 aa, Artificial Sequence
  43-Wnt7a_Delta1-99_Delta301-349
GSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQYPYDVPDYA

SEQ ID NO: 44 - DNA, 35 bp, Artificial Sequence
  44-Wnt7a_Delta1-99_Delta301-349 F-Primer
aaggatccaccatggggagccgggaggctgcgttc

SEQ ID NO: 45 - DNA, 60 bp, Artificial Sequence; identical to SEQ ID NO: 39
  45-Wnt7a_Delta1-99_Delta301-349 R-Primer

SEQ ID NO: 46 - protein, 344 aa, Artificial Sequence
  46-Wnt7a_DeltaEBP*GSGS
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEGSGSLSYRKPMDTDLVYIEKSPNYCEEDPVTGSVGTQGRACNKTAPQASGCDLMCCGRGYNTHQYARVWQCNCKFHWCCYVKCNTCSERTEMYTCKYPYDVPDYA

SEQ ID NO: 47 - DNA, 69 bp, Artificial Sequence
  47-Wnt7a_DeltaEBP*GSGS F-Primer
vvcaacgaggccgttcacgtggagcctggttcaggttcactgtcgtaccgcaagcccatggacacggac

SEQ ID NO: 48 - DNA, 67 bp, Artificial Sequence
  48-Wnt7a_DeltaEBP*GSGS R-Primer
gtccgtgtccatgggcttgcggtacgacagtgaacctgaaccaggctccacgtgaacggcctcgttg

SEQ ID NO: 49 - protein, 309 aa, Artificial Sequence
  49-Wnt7a_Delta1-49
AICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCKYPYDVPDYA

SEQ ID NO: 50 - DNA, 38 bp, Artificial Sequence
  50-Wnt7a_Delta1-49 F-Primer
aaggatccaccatggcgatctgccagagccggcccgac

SEQ ID NO: 51 - DNA, 58 bp, Artificial Sequence; identical to SEQ ID NO: 21
  51-Wnt7a_Delta1-49 R-Primer

SEQ ID NO: 52 - protein, 259 aa, Artificial Sequence
  52-Wnt7a_Delta1-99
GSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCKYPYDVPDYA

SEQ ID NO: 53 - DNA, 35 bp, Artificial Sequence; identical to SEQ ID NO: 44
  53-Wnt7a_Delta1-99 F-Primer

SEQ ID NO: 54 - DNA, 58 bp, Artificial Sequence; identical to SEQ ID NO: 21
  54-Wnt7a_Delta1-99 R-Primer

SEQ ID NO: 55 - protein, 209 aa, Artificial Sequence
  55-Wnt7a_Delta1-149
GGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCKYPYDVPDYA

SEQ ID NO: 56 - DNA, 35 bp, Artificial Sequence
  56-Wnt7a_Delta1-149 F-Primer
aaggatccaccatgggtggctgctctgccgacatc

SEQ ID NO: 57 - DNA, 58 bp, Artificial Sequence; identical to SEQ ID NO: 21
  57-Wnt7a_Delta1-149 R-Primer

SEQ ID NO: 58 - protein, 358 aa, Artificial Sequence
  58-Wnt7a_Delta3aa*GSG
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREGSGNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCKYPYDVPDYA

SEQ ID NO: 59 - DNA, 61 bp, Artificial Sequence
  59-Wnt7a_Delta3aa*GSG F-Primer
caaggtctttgtggatgcccgggagggctcggggaatgcccggactctcatgaacttgcac

SEQ ID NO: 60 - DNA, 61 bp, Artificial Sequence
  60-Wnt7a_Delta3aa*GSG R-Primer
gtgcaagttcatgagagtccgggcattccccgagccctcccgggcatccacaaagaccttg

SEQ ID NO: 61 - protein, 373 aa, Artificial Sequence
  61-Wnt7a_Delta3aa*ESP
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREPVRASRNKRPTFLKIKKPNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCKYPYDVPDYA

SEQ ID NO: 62 - DNA, 79 bp, Artificial Sequence
  62-Wnt7a_Delta3aa*ESP F-Primer
tggcttcttgatcttcaggaaggtgggccgcttgttgcggctggcacgcacaggctcccgggcatccacaaagaccttg

SEQ ID NO: 63 - DNA, 79 bp, Artificial Sequence
  63-Wnt7a_Delta3aa*ESP R-Primer
cctgtgcgtgccagccgcaacaagcggcccaccttcctgaagatcaagaagccaaatgcccggactctcatgaacttgc

SEQ ID NO: 64 - protein, 236 aa, Artificial Sequence
  64-Wnt7a_Delta213-349*ESP@172
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREPVRASRNKRPTFLKIKKPNARTLMNLHNNEAGRKILEENMKLECKCHGVSGSCTTKYPYDVPDYA

SEQ ID NO: 65 - DNA, 34 bp, Artificial Sequence; identical to SEQ ID NO: 32
  65-Wnt7a_Delta213-349*ESP@172 F-Primer

SEQ ID NO: 66 - DNA, 60 bp, Artificial Sequence; identical to SEQ ID NO: 33
  66-Wnt7a_Delta213-349*ESP@172 R-Primer

SEQ ID NO: 67 - protein, 239 aa, Artificial Sequence
  67-Wnt7a_Delta213-349*ESP
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKPVRASRNKRPTFLKIKKPYPYDVPDYA

SEQ ID NO: 68 - DNA, 34 bp, Artificial Sequence; identical to SEQ ID NO: 32
  68-Wnt7a_Delta213-349*ESP F-Primer

SEQ ID NO: 69 - DNA, 58 bp, Artificial Sequence
  69-Wnt7a_Delta213-349*ESP R1-Primer
gcttgttgcggctggcacgcacaggtcccgatcccttggtggtgcacgagcctgacac

SEQ ID NO: 70 - DNA, 88 bp, Artificial Sequence
  70-Wnt7a_Delta213-349*ESP R2-Primer
agaattctcaagcgtaatctggaacatcgtatgggtatggcttcttgatcttcaggaaggtgggccgcttgttgcggctggcacgcac

SEQ ID NO: 71 - protein, 239 aa, Artificial Sequence
  71-Wnt7a_*ESPDelta213-349
PVRASRNKRPTFLKIKKPMNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKYPYDVPDYA

SEQ ID NO: 72 - DNA, 59 bp, Artificial Sequence
  72-Wnt7a_*ESPDelta213-349 F1-Primer
cccaccttcctgaagatcaagaagccaggatcgggaatgaaccggaaagcgcggcgctg

SEQ ID NO: 73 - DNA, 61 bp, Artificial Sequence
  73-Wnt7a_*ESPDelta213-349 F2-Primer
aggatccccaatgcctgtgcgtgccagccgcaacaagcggcccaccttcctgaagatcaag

SEQ ID NO: 74 - DNA, 60 bp, Artificial Sequence; identical to SEQ ID NO: 33
  74-Wnt7a_*ESPDelta213-349 R1-Primer

SEQ ID NO: 75 - protein, 327 aa, Artificial Sequence
  75-HALO*ESP-HA
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGSGPVRASRNKRPTFLKIKKPYPYDVPDYA

SEQ ID NO: 76 - DNA, 47 bp, Artificial Sequence
  76-HALO*ESP-HA F-Primer
atataagcttaccatgatataagcttatggaggatctgtactttcag

SEQ ID NO: 77 - DNA, 72 bp, Artificial Sequence
  77-HALO*ESP-HA R-Primer
caagcggcccaccttcctgaagatcaagaagccatacccatacgatgttccagattacgcttgagaattctt

SEQ ID NO: 78 - protein, 318 aa, Artificial Sequence
  78-HALO*ESP
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGSGPVRASRNKRPTFLKIKKP

SEQ ID NO: 79 - DNA, 47 bp, Artificial Sequence; identical to SEQ ID NO: 76
  79-HALO*ESP F-Primer

SEQ ID NO: 80 - DNA, 71 bp, Artificial Sequence
  80-HALO*ESP R-Primer
cttcaggaaggtgggccgcttgttgcggctggcacgcacaggtcccgatccaccggaaatctccagagtag

SEQ ID NO: 81 - protein, 604 aa, Artificial Sequence
  81-Wnt7a-BirA-myc
MNRKARRCLGHLFFSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFQWCCYVKCNKCSERTEMYTCKTGGSGSGSGSDFKNLIWLKEVDSTQERLKEWNVSYGTALVADRQTKGRGGLGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEKSFKEFKGKIESKMLYLGEEVKLLGEGKITGKLVGLSEKGGALILTEEGIKEILSGEFSLRRSLEEQKLISEEDL

SEQ ID NO: 82 - DNA, 41 bp, Artificial Sequence
  82-Wnt7a-BirA-myc F1-Primer
tatagaattcgccaccatgaaccggaaagcgcggcgctgcc

SEQ ID NO: 83 - DNA, 58 bp, Artificial Sequence
  83-Wnt7a-BirA-myc F2-Primer
tataaccggtggaagtggaagtggaagtggaagtgacttcaagaacctgatctggctg

SEQ ID NO: 84 - DNA, 53 bp, Artificial Sequence
  84-Wnt7a-BirA-myc R1-Primer
tatagtcgaccatatgtatataaccggtcttgcacgtgtacatctccgtgcgc

SEQ ID NO: 85 - DNA, 65 bp, Artificial Sequence
  85-Wnt7a-BirA-myc R2-Primer
tatacatatgtcaaagatcttcctcggatatgagtttctgctcctcgaggcttcttctcaggctg

SEQ ID NO: 86 - protein, 267 aa, Artificial Sequence
  86-Myc-BirA-ESP
MEQKLISEEDLDFKNLIWLKEVDSTQERLKEWNVSYGTALVADRQTKGRGGLGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEKSFKEFKGKIESKMLYLGEEVKLLGEGKITGKLVGLSEKGGALILTEEGIKEILSGEFSLRRSLEGSGPVRASRNKRPTFLKIKKP

SEQ ID NO: 87 - DNA, 40 bp, Artificial Sequence
  87-Myc-BirA-ESP F-Primer
tatactcgagggatcgggacctgtgcgtgccagccgcaac

SEQ ID NO: 88 - DNA, 31 bp, Artificial Sequence
  88-Myc-BirA-ESP R-Primer
gtcgactcatggcttcttgatcttcaggaag

SEQ ID NO: 89 - protein, 358 aa, Artificial Sequence
  89-Wnt7a_ESP*Scramb
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPNKKLASPRITFKPKRRVLSYRKPMDTDLVYIEKSPNYCEEDPVTGSVGTQGRACNKTAPQASGCDLMCCGRGYNTHQYARVWQCNCKFHWCCYVKCNTCSERTEMYTCKYPYDVPDYA

SEQ ID NO: 90 - DNA, 62 bp, Artificial Sequence
  90-Wnt7a_ESP*Scramb F-Primer
tggcgagcccgcgcattacctttaaaccgaaacgccgcgtgctgtcgtaccgcaagcccatg

SEQ ID NO: 91 - DNA, 63 bp, Artificial Sequence
  91-Wnt7a_ESP*Scramb R-Primer
ggtttaaaggtaatgcgcgggctcgccagttttttgttcggctccacgtgaacggcctcgttg

SEQ ID NO: 92 - protein, 358 aa, Artificial Sequence
  92-Wnt7a_K247A
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNARPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCKYPYDVPDYA

SEQ ID NO: 93 - DNA, 42 bp, Artificial Sequence
  93-Wnt7a_K247A F-Primer
gtgcgtgccagccgcaacgcgcggcccaccttcctgaagatc

SEQ ID NO: 94 - DNA, 42 bp, Artificial Sequence
  94-Wnt7a_K247A R-Primer
gatcttcaggaaggtgggccgcgcgttgcggctggcacgcac

SEQ ID NO: 95 - protein, 358 aa, Artificial Sequence
  95-Wnt7a_K253A
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLAIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCKYPYDVPDYA

SEQ ID NO: 96 - DNA, 43 bp, Artificial Sequence
  96-Wnt7a_K253A F-Primer
caagcggcccaccttcctggcgatcaagaagccactgtcgtac

SEQ ID NO: 97 - DNA, 43 bp, Artificial Sequence
  97-Wnt7a_K253A R-Primer
gtacgacagtggcttcttgatcgccaggaaggtgggccgcttg

SEQ ID NO: 98 - protein, 358 aa, Artificial Sequence
  98-Wnt7a_K255A
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIAKPLSYRKPMDTDLVYIEKSPNYCEEDPVTGSVGTQGRACNKTAPQASGCDLMCCGRGYNTHQYARVWQCNCKFHWCCYVKCNTCSERTEMYTCKYPYDVPDYA

SEQ ID NO: 99 - DNA, 45 bp, Artificial Sequence
  99-Wnt7a_K255A F-Primer
cggcccaccttcctgaagatcgcgaagccactgtcgtaccgcaag

SEQ ID NO: 100 - DNA, 45 bp, Artificial Sequence
  100-Wnt7a_K255A R-Primer
cttgcggtacgacagtggcttcgcgatcttcaggaaggtgggccg

SEQ ID NO: 101 - protein, 358 aa, Artificial Sequence
  101-Wnt7a_K256A
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKAPLSYRKPMDTDLVYIEKSPNYCEEDPVTGSVGTQGRACNKTAPQASGCDLMCCGRGYNTHQYARVWQCNCKFHWCCYVKCNTCSERTEMYTCKYPYDVPDYA

SEQ ID NO: 102 - DNA, 43 bp, Artificial Sequence
  102-Wnt7a_K256A F-Primer
caccttcctgaagatcaaggcgccactgtcgtaccgcaagccc

SEQ ID NO: 103 - DNA, 43 bp, Artificial Sequence
  103-Wnt7a_K256A R-Primer
gggcttgcggtacgacagtggcgccttgatcttcaggaaggtg

SEQ ID NO: 104 - protein, 364 aa, Artificial Sequence
  104-Wnt7a_DeltaESP*Wnt10a-ESP
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPHNRNGGQLEPGPAGAPSPAPGAPGPRRRASDTDLVYIEKSPNYCEEDPVTGSVGTQGRACNKTAPQASGCDLMCCGRGYNTHQYARVWQCNCKFHWCCYVKCNTCSERTEMYTCKYPYDVPDYA

SEQ ID NO: 105 - DNA, 34 bp, Artificial Sequence; identical to SEQ ID NO: 32
  105-Wnt7a_DeltaESP*Wnt10a-ESP F1-Primer

SEQ ID NO: 106 - DNA, 57 bp, Artificial Sequence
  106-Wnt7a_DeltaESP*Wnt10a-ESP F2-Primer
gctccgggcgctcccgggccgcgccgacgggccagcgacacggacctggtgtacatc

SEQ ID NO: 107 - DNA, 54 bp, Artificial Sequence
  107-Wnt7a_DeltaESP*Wnt10a-ESP F3-Primer
ctggagccgggcccagcgggggcaccctcgccggctccgggcgctcccgggccg

SEQ ID NO: 108 - DNA, 57 bp, Artificial Sequence
  108-Wnt7a_DeltaESP*Wnt10a-ESP R1-Primer
tgggcccggctccagctggccgccgttgcggttgtgaggctccacgtgaacggcctc

SEQ ID NO: 109 - DNA, 58 bp, Artificial Sequence; identical to SEQ ID NO: 21
  109-Wnt7a_DeltaESP*Wnt10a-ESP R2-Primer

SEQ ID NO: 110 - protein, 353 aa, Artificial Sequence
  110-Wnt7a_DeltaESP*Wnt16-ESP
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPKTKRKMRRREKDQRKIPIHDTDLVYIEKSPNYCEEDPVTGSVGTQGRACNKTAPQASGCDLMCCGRGYNTHQYARVWQCNCKFHWCCYVKCNTCSERTEMYTCKYPYDVPDYA

SEQ ID NO: 111 - DNA, 34 bp, Artificial Sequence; identical to SEQ ID NO: 32
  111-Wnt7a_DeltaESP*Wnt16-ESP F1-Primer

SEQ ID NO: 112 - DNA, 57 bp, Artificial Sequence
  112-Wnt7a_DeltaESP*Wnt16-ESP F2-Primer
aggagagaaaaagatcagaggaaaataccaatccatgacacggacctggtgtacatc

SEQ ID NO: 113 - DNA, 57 bp, Artificial Sequence
  113-Wnt7a_DeltaESP*Wnt16-ESP R1-Primer
atctttttctctcctgcgcattttcctctttgttttaggctccacgtgaacggcctc

SEQ ID NO: 114 - DNA, 58 bp, Artificial Sequence; identical to SEQ ID NO: 21
  114-Wnt7a_DeltaESP*Wnt16-ESP R2-Primer

SEQ ID NO: 115 - protein, 381 aa, Artificial Sequence
  115-Wnt10b_DeltaESP*GSGS
MLEEPRPRPPPSGLAGLLFLALCSRALSNEILGLKLPGEPPLTANTVCLTLSGLSKRQLGLCLRNPDVTASALQGLHIAVHECQHQLRDQRWNCSALEGGGRLPHHSAILKRGFRESAFSFSMLAAGVMHAVATACSLGKLVSCGCGWKGSGEQDRLRAKLLQLQALSRGKSFPHSLPSPGPGSSPSPGPQDTWEWGGCNHDMDFGEKFSRDFLDSREAPRDIQARMRIHNNRVGRQVVTENLKRKCKCHGTSGSCQFKTCWRAAPEFRAVGAALRERLGRAIFIGSGSELVYFEKSPDFCERDPTMGSPGTRGRACNKTSRLLDGCGSLCCGRGHNVLRQTRVERCHCRFHWCCYVLCDECKVTEWVNVCKYPYDVPDYA

SEQ ID NO: 116 - DNA, 31 bp, Artificial Sequence
  116-Wnt10b_DeltaESP*GSGS F1-Primer
aaggatccaccatgctggaggagccccggcc

SEQ ID NO: 117 - DNA, 61 bp, Artificial Sequence
  117-Wnt10b_DeltaESP*GSGS F2-Primer
cggctgggccgggccatcttcattggttcaggttcagagctggtctactttgagaagtctc

SEQ ID NO: 118 - DNA, 61 bp, Artificial Sequence
  118-Wnt10b_DeltaESP*GSGS R1-Primer
gagacttctcaaagtagaccagctctgaacctgaaccaatgaagatggcccggcccagccg

SEQ ID NO: 119 - DNA, 30 bp, Artificial Sequence
  119-Wnt10b_DeltaESP*GSGS R2-Primer
aagaattcctaaccggtacgcgtagaatcg

SEQ ID NO: 120 - protein, 398 aa, Artificial Sequence
  120-Wnt10b_RR302AA
MLEEPRPRPPPSGLAGLLFLALCSRALSNEILGLKLPGEPPLTANTVCLTLSGLSKRQLGLCLRNPDVTASALQGLHIAVHECQHQLRDQRWNCSALEGGGRLPHHSAILKRGFRESAFSFSMLAAGVMHAVATACSLGKLVSCGCGWKGSGEQDRLRAKLLQLQALSRGKSFPHSLPSPGPGSSPSPGPQDTWEWGGCNHDMDFGEKFSRDFLDSREAPRDIQARMRIHNNRVGRQVVTENLKRKCKCHGTSGSCQFKTCWRAAPEFRAVGAALRERLGRAIFIDTHNRNSGAFQPRLRPAALSGELVYFEKSPDFCERDPTMGSPGTRGRACNKTSRLLDGCGSLCCGRGHNVLRQTRVERCHCRFHWCCYVLCDECKVTEWVNVCKYPYDVPDYA

SEQ ID NO: 121 - DNA, 55 bp, Artificial Sequence
  121-Wnt10b_RR302AA F-Primer
gccttccagccccgtctgcgtcccgctgccctctcaggagagctggtctactttg

SEQ ID NO: 122 - DNA, 55 bp, Artificial Sequence
  122-Wnt10b_RR302AA R-Primer
caaagtagaccagctctcctgagagggcagcgggacgcagacggggctggaaggc